(19) United States
(12) Patent Application Publication
(10) Pub. No.: US 2023/0364137 A1
(43) Pub. Date: Nov. 16, 2023

(54) CHIMERIC ANTIGEN RECEPTOR EXPRESSION SYSTEMS

(71) Applicant: VOR BIOPHARMA INC., Cambridge, MA (US)

(72) Inventors: Sadik Kassim, Cambridge, MA (US); Tirtha Chakraborty, Cambridge, MA (US); Julian Scherer, Cambridge, MA (US)

(73) Assignee: VOR BIOPHARMA INC., Cambridge, MA (US)

(21) Appl. No.: 18/029,298

(22) PCT Filed: Sep. 30, 2021
(86) PCT No.: PCT/US2021/052990

§ 371 Date: Mar. 29, 2023

Related U.S. Application Data
(60) Provisional application No. 63/085,983, filed on Sep. 30, 2020.

(51) Int. Cl. A61K 35/17; C12N 15/63; C12N 15/86; A61K 48/00

(52) U.S. Cl. CPC A61K 35/17; C12N 15/635; C12N 15/86; A61K 48/0058; C12N 2740/16043; C12N 2830/003; C12N 2830/008; C12N 2830/48; C12N 2830/50

(57) ABSTRACT

Aspects of this disclosure provide nucleic acid constructs A nucleic acid construct, comprising a first expression cassette comprising a nucleic acid encoding a tetracycline-responsive transactivator under the control of a cell type-specific promoter; and a second expression cassette comprising a nucleic acid encoding a transgene, for example, a chimeric antigen receptor (CAR), under the control of a tetracycline-responsive promoter.

### RELATED APPLICATION

This application claims the benefit under 35 U.S.C. 119(e) of U.S. provisional application No. 63/085,983 filed Sep. 30, 2020, which is incorporated by reference herein in its entirety.

### BACKGROUND

Chimeric antigen receptors (CARs) mediate antigen recognition and targeting by cellular immunotherapies, such as CAR-T and CAR-NK cell therapies. There is a need for improved CAR expression systems driving the extent of CAR expression in therapeutic cells.

### SUMMARY

Some aspects of this disclosure provide nucleic acid constructs comprising a first expression cassette comprising a nucleic acid encoding a tetracycline-responsive transactivator under the control of a cell type-specific promoter; and a second expression cassette comprising a nucleic acid encoding a transgene, e.g., a chimeric antigen receptor, under the control of a tetracycline-responsive promoter. In some embodiments, the transgene encodes a chimeric antigen receptor targeting a human antigen associated with a disease or disorder. In some embodiments, the cell type-specific promoter is a constitutive promoter. In some embodiments, the cell type specific promoter is a CD8 promoter. In some embodiments, the cells type-specific promoter is a CD3delta promoter. In some embodiments, the cell type-specific promoter is a CD56 promoter. In some embodiments, the cell type-specific promoter is a CD244 promoter. In some embodiments, the cell type-specific promoter is a CD94 promoter. In some embodiments, the cell type-specific promoter is an NKG2D promoter. In some embodiments, the cell type-specific promoter is a CD3D promoter. In some embodiments, the cell type-specific promoter is a CD3E promoter. In some embodiments, the tetracycline-responsive transactivator is an rtTA protein. In some embodiments, the tetracycline-responsive transactivator is an rtTA protein. In some embodiments, the tetracycline-responsive transactivator is an rtTA2^{S}-M2 protein. In some embodiments, the tetracycline-responsive transactivator is an rtTA-V10 protein. In some embodiments, the tetracycline-responsive transactivator is an rtTA-V16 protein. In some embodiments, the tetracycline-responsive transactivator is an rtTA-3G protein. In some embodiments, the transgene encodes a chimeric antigen receptor (CAR) comprising a binding domain, a hinge domain, a transmembrane domain, at least one co-stimulatory domain, a cytoplasmic signaling domain, or a combination thereof. In some embodiments, the binding domain comprises an antibody, or an antigen-binding antibody fragment, that binds an antigen. In some embodiments, the binding domain comprises an scFv or a single domain antibody that binds to an antigen. In some embodiments, the antigen is a lineage-specific cell-surface antigen. In some embodiments, expression of the antigen is associated with a neoplastic or malignant disease. In some embodiments, the antigen is CD33, CD123, CD19, or CLL-1. In some embodiments, the hinge domain of the CAR is a CD8a (CD8alpha) hinge domain. In some embodiments, the transmembrane domain of the CAR is a CD8 or CD28 transmembrane domain. In some embodiments, the costimulatory domain of the CAR is a 4-1BB or CD28 costimulatory domain, or a combination thereof. In some embodiments, the cytoplasmic signaling domain of the CAR is a CD3 (CD3zeta) cytoplasmic signaling domain.

Some aspects of this disclosure provide cells, e.g., immune effector cells or hematopoietic stem or progenitor cells, comprising a nucleic acid constructs comprising a first expression cassette comprising a nucleic acid encoding a tetracycline-responsive transactivator under the control of a cell type-specific promoter; and a second expression cassette comprising a nucleic acid encoding a transgene, e.g., a chimeric antigen receptor, under the control of a tetracycline-responsive promoter. In some embodiments, the transgene encodes a chimeric antigen receptor targeting a human antigen associated with a disease or disorder. In some embodiments, the cell type-specific promoter is a constitutive promoter. In some embodiments, the cell type specific promoter is a CD8 promoter. In some embodiments, the cells type-specific promoter is a CD3delta promoter. In some embodiments, the cell type-specific promoter is a CD56 promoter. In some embodiments, the cell type-specific promoter is a CD244 promoter. In some embodiments, the cell type-specific promoter is a CD94 promoter. In some embodiments, the cell type-specific promoter is an NKG2D promoter. In some embodiments, the cell type-specific promoter is a CD3D promoter. In some embodiments, the cell type-specific promoter is a CD3E promoter. In some embodiments, the tetracycline-responsive transactivator is an rtTA protein. In some embodiments, the tetracycline-responsive transactivator is an rtTA protein. In some embodiments, the tetracycline-responsive transactivator is an rtTA2^{S}-M2 protein. In some embodiments, the tetracycline-responsive transactivator is an rtTA-V10 protein. In some embodiments, the tetracycline-responsive transactivator is an rtTA-V16 protein. In some embodiments, the tetracycline-responsive transactivator is an rtTA-3G protein. In some embodiments, the transgene encodes a chimeric antigen receptor comprising a binding domain, a hinge domain, a transmembrane domain, at least one co-stimulatory domain, a cytoplasmic signaling domain, or a combination thereof. In some embodiments, the binding domain comprises an antibody, or an antigen-binding antibody fragment, that binds an antigen. In some embodiments, the binding domain comprises an scFv or a single domain antibody that binds to an antigen. In some embodiments, the antigen is a lineage-specific cell-surface antigen. In some embodiments, expression of the antigen is associated with a neoplastic or malignant disease. In some embodiments, the antigen is CD33, CD123, CD19, or CLL-1. In some embodiments, the hinge domain of the CAR is a CD8a (CD8alpha) hinge domain. In some embodiments, the transmembrane domain of the CAR is a CD8 or CD28 transmembrane domain. In some embodiments, the costimulatory domain of the CAR is a 4-1BB or CD28 costimulatory domain, or a combination thereof. In some embodiments, the cytoplasmic signaling domain of the CAR is a CD3 (CD3zeta) cytoplasmic signaling domain. In some embodiments, the cells are hematopoietic stem cells. In some embodiments, the cells are hematopoietic progenitor cells. In some embodiments, the cells are immune effector cells. In some embodiments, the cells are T-cells. In some embodiments, the cells are alpha/beta T-cells. In some embodiments, the cells are gamma/delta T-cells. In some embodiments, the cells are natural killer (NK) cells. In some embodiments, the cells are natural killer T-cells (NKT cells).

Some aspects of this disclosure provide methods, comprising administering a plurality of cells provided herein to a subject in need thereof. For example, some aspects of this disclosure provide methods comprising administering a plurality, and preferably an effective number, of cells provided herein, e.g., immune effector cells or hematopoietic stem or progenitor cells that comprise a nucleic acid constructs comprising a first expression cassette comprising a nucleic acid encoding a tetracycline-responsive transactivator under the control of a cell type-specific promoter; and a second expression cassette comprising a nucleic acid encoding a transgene, e.g., a chimeric antigen receptor, under the control of a tetracycline-responsive promoter, to a subject in need thereof. In some embodiments, the subject has been diagnosed or has a neoplastic or malignant disease and the transgene encoded by the nucleic acid construct comprised in the cell is a CAR targeting an antigen associated with the neoplastic or malignant disease. In some embodiments, the cells are CAR-T cells. In some embodiments, the cells are CAR-NK cells. In some embodiments, the cells are HSCs or HPCs. In some embodiments, the methods further comprise administering to the subject an effective amount of tetracycline resulting in induction of the expression of the transgene. In some embodiments, the methods further comprise measuring expression of the transgene in a cell obtained from the subject. In some embodiments, the methods further comprise monitoring at least one symptom of the neoplastic or malignant disease and continuing administration of the tetracycline until the at least one symptom of the neoplastic or malignant disease is ameliorated.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIGS. 1A-1C. Schematics illustrating exemplary embodiments of nucleic acid constructs comprising an expression cassette encoding a transactivator under the control of a cell type-specific promoter, and an expression cassette encoding a transgene, e.g., a CAR, under the control of a transactivator-responsive promoter. Transcription start sites and direction of transcription are indicated by black arrows. FIG. 1A illustrates an exemplary configuration of a nucleic acid construct, in which both expression cassettes are comprised in one nucleic acid molecule. FIG. 1B illustrates an exemplary configuration of a nucleic acid construct, in which both expression cassettes are comprised in one nucleic acid molecule but in reverse order. FIG. 1C illustrates an exemplary configuration of a nucleic acid construct, in which the expression cassettes are oriented in opposite directions. FIG. 1D illustrates an exemplary configuration of a nucleic acid construct, in which both expression cassettes are comprised in different nucleic acid molecules, e.g., on different vector molecules or on different chromosomes in the DNA of a cell.

FIGS. 2A-2C. Schematic illustrations of transactivator and transgene expression in different scenarios. Transcription start sites and direction of transcription are indicated by black arrows. Short black arrows indicate no active transcription, while long black arrows indicate transcription (and thus expression) of the respective coding sequence (e.g., encoding the transactivator or the transgene). Ovals represent encoded products, e.g., rTTA protein or transgene protein (e.g., CAR protein). FIG. 2A illustrates a scenario in a cell in which the cell type-specific promoter is not active. Both expression cassettes are inactive, neither the sequence encoding the transactivator nor the sequence encoding the transgene are transcribed. FIG. 2B illustrates a scenario in a cell in which the cell type-specific promoter is active, but a small molecule activating the transactivator (e.g., tetracycline, doxycycline, or an equivalent in the case of the transactivator being rTTA) is absent. The cell type-specific promoter initiates transcription and thus expression of the transactivator (e.g., rTTA), resulting in the expression of rTTA protein. In the absence of the small molecule activating the transactivator (e.g., tetracycline, doxycycline, or an equivalent in the case of the transactivator being rTTA), the transactivator does not bind the transactivator-responsive promoter, which is thus inactive, and no expression of the transgene (e.g., a CAR) occurs. FIG. 2C illustrates a scenario in a cell in which the cell type-specific promoter is active and the small molecule activating the transactivator is present. The cell type-specific promoter initiates transcription and thus expression of the transactivator (e.g., rTTA), resulting in the expression of rTTA protein. In the presence of the small molecule activating the transactivator (e.g., tetracycline, doxycycline, or an equivalent in the case of the transactivator being rTTA), the transactivator binds and activates the transactivator-responsive promoter, which results in transcription of the nucleic acid sequence encoding the transgene (e.g., the CAR), and subsequent expression of transgene protein (e.g., CAR protein).

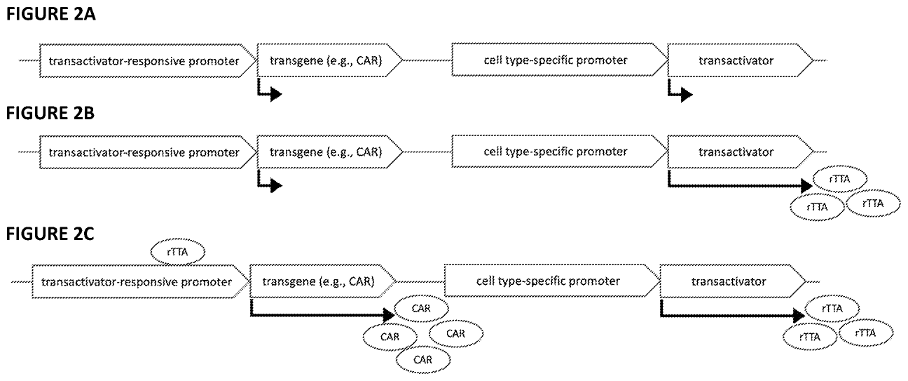

FIGS. 3A-3C. Schematic illustrations of transactivator and transgene expression in different scenarios. Transcription start sites and direction of transcription are indicated by black arrows. Short black arrows indicate no active transcription, while long black arrows indicate transcription (and thus expression) of the respective coding sequence (e.g., encoding the rTTA or the CAR). Ovals represent encoded products, e.g., rTTA protein or CAR protein. FIG. 3A illustrates a scenario in a cell in which the T-cell specific CD8 promoter (pCD8) is not active, e.g., in cells of non-hematopoietic lineages, or in hematopoietic cells outside of the T-cell lineage. Both expression cassettes are inactive, neither the sequence encoding the rTTA nor the sequence encoding the CAR are transcribed. FIG. 3B illustrates a scenario in a cell in which the CD8 promoter is active, e.g., in pre-T-cells or in mature T-cells, but the small molecule activating the transactivator (e.g., tetracycline, doxycycline, or an equivalent in the case of the transactivator being rTTA) is absent. The CD8 promoter initiates transcription and thus expression of the transactivator (e.g., rTTA), resulting in the expression of rTTA protein. In the absence of the small molecule activating the transactivator, the transactivator does not bind the transactivator-responsive promoter, which is thus inactive, and no expression of the CAR occurs. FIG. 3C illustrates a scenario in a cell in which the CD8 promoter is active, e.g., in pre-T-cells or in mature T-cells, and the small molecule activating the transactivator (e.g., tetracycline, doxycycline, or equivalent) is present. The CD8 promoter initiates transcription and thus expression of the nucleic acid encoding the rTTA, resulting in the expression of rTTA protein. In the presence of the small molecule activating the transactivator, the transactivator binds and activates the transactivator-responsive promoter, which results in transcription of the nucleic acid sequence encoding the CAR, and subsequent expression of CAR protein.

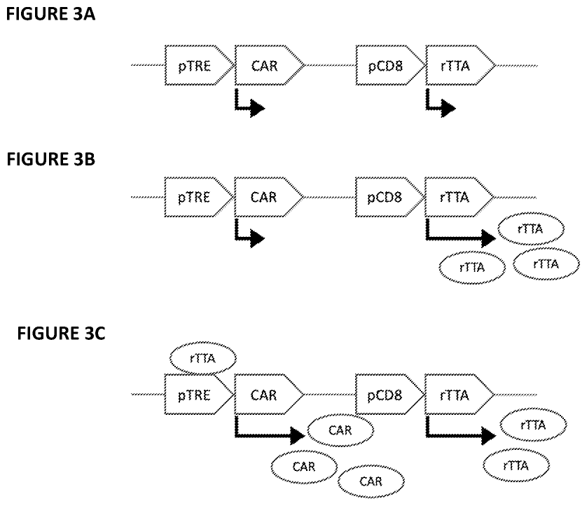

FIGS. 4A-4C. Schematic illustrations of transactivator and transgene expression in different scenarios. Transcription start sites and direction of transcription are indicated by black arrows. Short black arrows indicate no active transcription, while long black arrows indicate transcription (and thus expression) of the respective coding sequence (e.g., encoding the rTTA or the CAR). Ovals represent encoded products, e.g., rTTA protein or CAR protein. FIG. 4A illustrates a scenario in a cell in which the T-cell specific T-cell receptor beta promoter (pTCRb) is not active, e.g., in cells of non-hematopoietic lineages, or in hematopoietic cells outside of the T-cell lineage. Both expression cassettes are inactive, neither the sequence encoding the rTTA nor the sequence encoding the CAR are transcribed. FIG. 4B illustrates a scenario in a cell in which the TCRb promoter is active, e.g., in pre-T-cells or in mature T-cells, but the small molecule activating the transactivator (e.g., tetracycline, doxycycline, or an equivalent in the case of the transactivator being rTTA) is absent. The TCRb promoter initiates transcription and thus expression of the transactivator (e.g., rTTA), resulting in the expression of rTTA protein. In the absence of the small molecule activating the transactivator, the transactivator does not bind the transactivator-responsive promoter, which is thus inactive, and no expression of the CAR occurs. FIG. 4C illustrates a scenario in a cell in which the TCRb promoter is active, e.g., in pre-T-cells or in mature T-cells, and the small molecule activating the transactivator (e.g., tetracycline, doxycycline, or equivalent) is present. The TCRb promoter initiates transcription and thus expression of the nucleic acid encoding the rTTA, resulting in the expression of rTTA protein. In the presence of the small molecule activating the transactivator, the transactivator binds and activates the transactivator-responsive promoter, which results in transcription of the nucleic acid sequence encoding the CAR, and subsequent expression of CAR protein.

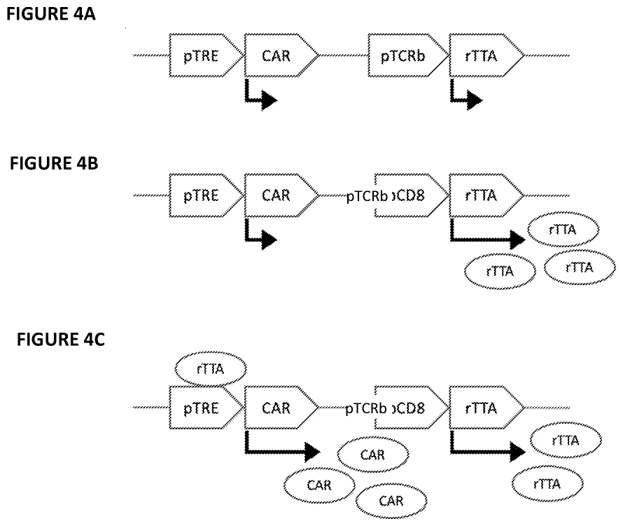

FIGS. 5A-5C. Schematic illustrations of transactivator and transgene expression in different scenarios. Transcription start sites and direction of transcription are indicated by black arrows. Short black arrows indicate no active transcription, while long black arrows indicate transcription (and thus expression) of the respective coding sequence (e.g., encoding the rTTA or the CAR). Ovals represent encoded products, e.g., rTTA protein or CAR protein. FIG. 5A illustrates a scenario in a cell in which the T-cell specific CD3D promoter (pCD3D) is not active, e.g., in cells of non-hematopoietic lineages, or in hematopoietic cells outside of the T-cell lineage. Both expression cassettes are inactive, neither the sequence encoding the rTTA nor the sequence encoding the CAR are transcribed. FIG. 5B illustrates a scenario in a cell in which the CD3D promoter is active, e.g., in mature T-cells, but the small molecule activating the transactivator (e.g., tetracycline, doxycycline, or an equivalent in the case of the transactivator being rTTA) is absent. The CD3D promoter initiates transcription and thus expression of the transactivator (e.g., rTTA), resulting in the expression of rTTA protein. In the absence of the small molecule activating the transactivator, the transactivator does not bind the transactivator-responsive promoter, which is thus inactive, and no expression of the CAR occurs. FIG. 5C illustrates a scenario in a cell in which the CD3D promoter is active, e.g., in mature T-cells, and the small molecule activating the transactivator (e.g., tetracycline, doxycycline, or equivalent) is present. The CD3D promoter initiates transcription and thus expression of the nucleic acid encoding the rTTA, resulting in the expression of rTTA protein. In the presence of the small molecule activating the transactivator, the transactivator binds and activates the transactivator-responsive promoter, which results in transcription of the nucleic acid sequence encoding the CAR, and subsequent expression of CAR protein.

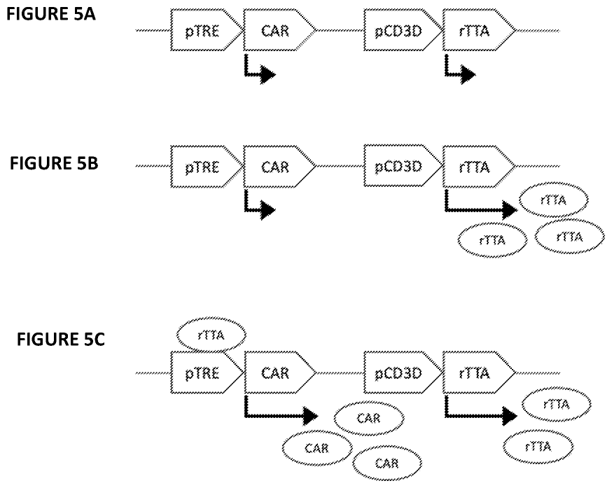

FIGS. 6A-6C. Schematic illustrations of transactivator and transgene expression in different scenarios. Transcription start sites and direction of transcription are indicated by black arrows. Short black arrows indicate no active transcription, while long black arrows indicate transcription (and thus expression) of the respective coding sequence (e.g., encoding the rTTA or the CAR). Ovals represent encoded products, e.g., rTTA protein or CAR protein. FIG. 6A illustrates a scenario in a cell in which the natural killer cell specific NKG2D promoter (pNKG2D) is not active, e.g., in cells of non-hematopoietic lineages, or in hematopoietic cells outside of the NK-cell lineage. Both expression cassettes are inactive, neither the sequence encoding the rTTA nor the sequence encoding the CAR are transcribed. FIG. 6B illustrates a scenario in a cell in which the NKG2D promoter is active, e.g., in mature NK-cells, but the small molecule activating the transactivator (e.g., tetracycline, doxycycline, or an equivalent in the case of the transactivator being rTTA) is absent. The NKG2D promoter initiates transcription and thus expression of the transactivator (e.g., rTTA), resulting in the expression of rTTA protein. In the absence of the small molecule activating the transactivator, the transactivator does not bind the transactivator-responsive promoter, which is thus inactive, and no expression of the CAR occurs. FIG. 6C illustrates a scenario in a cell in which the NKG2D promoter is active, e.g., in mature NK-cells, and the small molecule activating the transactivator (e.g., tetracycline, doxycycline, or equivalent) is present. The NKG2D promoter initiates transcription and thus expression of the nucleic acid encoding the rTTA, resulting in the expression of rTTA protein. In the presence of the small molecule activating the transactivator, the transactivator binds and activates the transactivator-responsive promoter, which results in transcription of the nucleic acid sequence encoding the CAR, and subsequent expression of CAR protein.

FIGS. 7A-7C. Schematics of exemplary lentiviral expression constructs.

FIGS. 8A-8C. Schematics of exemplary lentiviral expression constructs.

FIGS. 9A-9C. Schematics of exemplary lentiviral expression constructs.

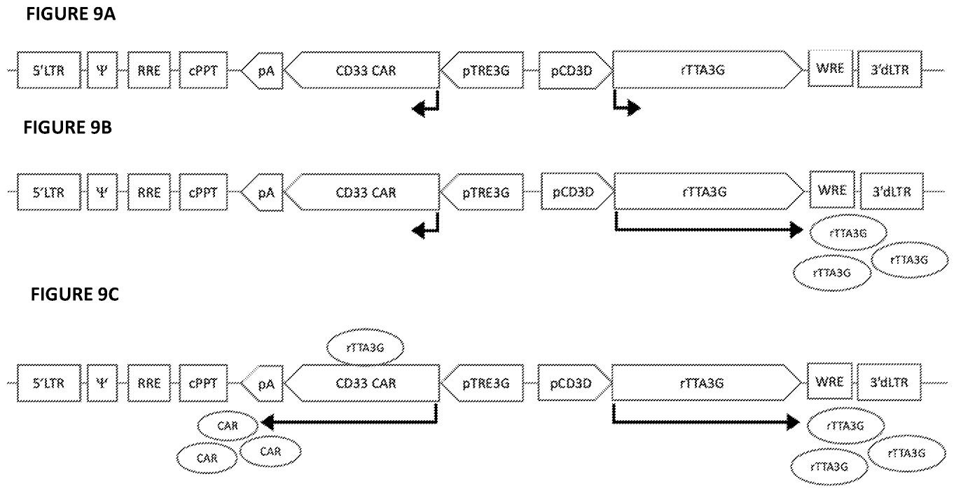

FIGS. 10A-10C. Schematics of exemplary lentiviral expression constructs.

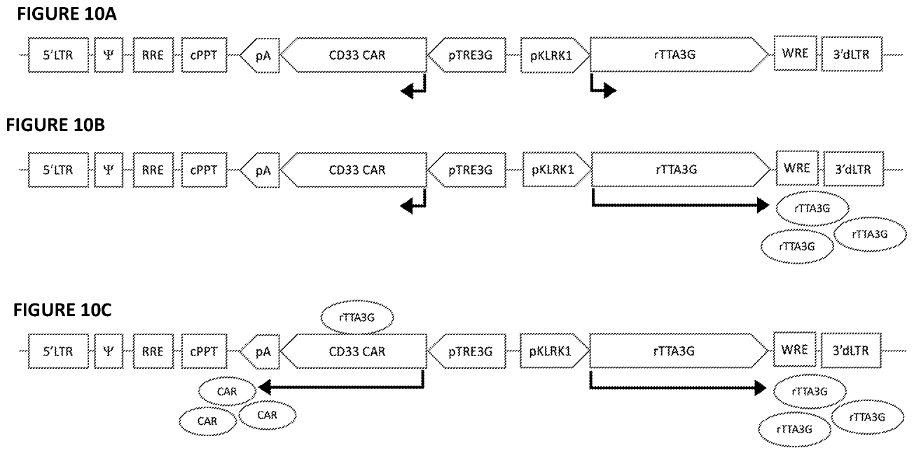

FIG. 11. Graphs of flow cytometric analysis data showing the number of Jurkat or Raji cells expressing the CAR (CAR positive cells) after transduction with lentiviral constructs encoding an exemplary CD33 CAR construct under control of an exemplary CD3D promoter.

### DEFINITIONS

Agent: As used herein, the term “agent” (or “biological agent” or “therapeutic agent”), refers to a molecule that may be expressed, released, secreted or delivered to a target by a modified cell (e.g., an immune cell comprising a transgene encoding a chimeric antigen receptor) provided herein. Examples of an agent include, but are not limited to, a nucleic acid, an antibiotic, an anti-inflammatory agent, an antibody or fragments thereof, a chimeric antigen receptor, an antibody agent or fragments thereof, a growth factor, a cytokine, an enzyme, a protein, a peptide, a fusion protein, a synthetic molecule, an organic molecule (e.g., a small molecule), a carbohydrate, a lipid, a hormone, a microsome, a derivative or a variation thereof, and any combinations thereof. An agent may bind any cell moiety, such as a receptor, an antigenic determinant, or other binding site present on a target or target cell. An agent may diffuse or be transported into a cell, where it may act intracellularly.

Antibody: As used herein, the term “antibody” refers to a polypeptide that includes canonical immunoglobulin sequence elements sufficient to confer specific binding to a particular target antigen. As is known in the art, intact antibodies as produced in nature are typically approximately 150 kD tetrameric agents comprising two identical heavy chain polypeptides (about 50 kD each) and two identical light chain polypeptides (about 25 kD each) that associate with each other into what is commonly referred to as a “Y-shaped” structure. Each heavy chain comprises at least four domains (each about 110 amino acids long)—an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain comprises two domains—an amino-terminal variable (VL) domain, followed by a carboxy-terminal constant (CL) domain, separated from one another by another “switch”. Intact antibody tetramers comprise two heavy chain-light chain dimers in which the heavy and light chains are linked to one another by a single disulfide bond; two other disulfide bonds connect the heavy chain hinge regions to one another, so that the dimers are connected to one another and a tetramer is formed. Naturally-produced antibodies are also typically glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complementarity determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including, for example, effector cells that mediate cytotoxicity. Affinity and/or other binding attributes of Fc regions for Fc receptors can be modulated through glycosylation or other modification. In some embodiments, antibodies produced and/or utilized in accordance with the present invention (e.g., as a component of a CAR) include glycosylated Fc domains, including Fc domains with modified or engineered glycosylation. In some embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal. In some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc., as is known in the art. Moreover, the term “antibody”, as used herein, can refer in appropriate embodiments (unless otherwise stated or clear from context) to any of the art-known or developed constructs or formats for utilizing antibody structural and functional features in alternative presentation. For example, in some embodiments, an antibody utilized in accordance with the present invention is in a format selected from, but not limited to, intact IgA, IgG, IgE or IgM antibodies; bi- or multi-specific antibodies (e.g., Zybodies®, etc); antibody fragments such as is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity). An antibody described herein can be an immunoglobulin, heavy chain antibody, light chain antibody, LRR-based antibody, or other protein scaffold with antibody-like properties, as well as other immunological binding moiety known in the art, including, e.g., a Fab, Fab′, Fab′2, Fab2, Fab3, F(ab′)2, Fd, Fv, Feb, scFv, SMIP, single domain antibody, single-chain antibody, diabody, triabody, tetrabody, minibody, maxibody, tandab, DVD, BiTe, TandAb, or the like, or any combination thereof. The subunit structures and three-dimensional configurations of different classes of antibodies are known in the art. In some embodiments, an antibody may lack a covalent modification (e.g., attachment of a glycan) that it would have if produced naturally. In some embodiments, an antibody may contain a covalent modification (e.g., attachment of a glycan, a payload (e.g., a detectable moiety, a therapeutic moiety, a catalytic moiety, etc.), or other pendant group (e.g., poly-ethylene glycol, etc.).

Antigen-binding fragment: An “antigen-binding fragment” refers to a portion of an antibody that binds the antigen to which the antibody binds. An antigen-binding fragment of an antibody includes any naturally occurring, enzymatically obtainable, synthetic, or genetically engineered polypeptide or glycoprotein that specifically binds an antigen to form a complex. Exemplary antibody fragments include, but are not limited to, Fv, Fab, Fab′, Fab′-SH, F(ab′)2; diabodies; single domain antibodies; linear antibodies; single-chain antibody molecules (e.g. scFv or VHH or VH or VL domains only); and multispecific antibodies formed from antibody fragments. In some embodiments, the antigen-binding fragments of the antibodies described herein are scFvs. In some embodiments, the antigen-binding fragments of the antibodies described herein are VHH domains only. As with full antibody molecules, antigen-binding fragments may be mono-specific or multispecific (e.g., bispecific). A multispecific antigen-binding fragment of an antibody may comprise at least two different variable domains, wherein each variable domain is capable of specifically binding to a separate antigen or to a different epitope of the same antigen.

Antibody heavy chain: As used herein, the term “antibody heavy chain” refers to the larger of the two types of polypeptide chains present in all antibody molecules in their naturally occurring conformations.

Antibody light chain: As used herein, the term “antibody light chain” refers to the smaller of the two types of polypeptide chains present in all antibody molecules in their naturally occurring conformations.

Synthetic antibody: As used herein, the term “synthetic antibody” refers to an antibody that is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

Antigen: As used herein, the term “antigen” or “Ag” refers to a molecule that is capable of provoking an immune response. This immune response may involve either antibody production, the activation of specific immunologically-competent cells, or both. A skilled artisan will understand that any macromolecule, including virtually all proteins or peptides, can serve as an antigen. Furthermore, antigens can be derived from recombinant or genomic DNA. A skilled artisan will understand that any DNA that comprises a nucleotide sequences or a partial nucleotide sequence encoding a protein that elicits an immune response encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

Autologous: As used herein, the term “autologous” refers to any material derived from an individual to which it is later to be re-introduced into the same individual.

Allogeneic: As used herein, the term “allogeneic” refers to any material (e.g., a population of cells) derived from a different animal of the same species.

Neoplastic disease: As used herein, the term “neoplastic disease” refers to a disease characterized by the rapid and uncontrolled growth of aberrant cells. A neoplastic disease may be a benign or a malign disease. Malign diseases are typically characterized by the presence of malign cells, e.g., cancer cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is acute myeloid leukemia. In some embodiments, the neoplastic disease is myelodysplastic syndrome.

Conservative sequence modifications: As used herein, the term “conservative sequence modifications” refers to amino acid modifications that do not significantly affect or alter the binding characteristics of an antibody containing the amino acid sequence. Such conservative modifications include amino acid substitutions, additions and deletions. Modifications can be introduced into an antibody compatible with various embodiments by standard techniques known in the art, such as site-directed mutagenesis and PCR-mediated mutagenesis. Conservative amino acid substitutions are ones in which an amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

Co-stimulatory ligand: As used herein, the term “co-stimulatory ligand” refers to a molecule on an antigen presenting cell (e.g., an APC, dendritic cell, B cell, and the like) that specifically binds a cognate co-stimulatory molecule on an immune cell (e.g., a T lymphocyte), thereby providing a signal which mediates an immune cell response, including, but not limited to, proliferation, activation, differentiation, and the like. A co-stimulatory ligand can include, but is not limited to, CD7, B7-1 (CD80), B7-2 (CD86), CD28, PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on an immune cell (e.g., a T lymphocyte), such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

Cytotoxic: As used herein, the term “cytotoxic” or “cytotoxicity” refers to killing or damaging cells. In some embodiments, cytotoxicity of the metabolically enhanced cells is improved, e.g. increased cytolytic activity of immune cells (e.g., T lymphocytes).

Effective amount: As used herein, an “effective amount” as described herein refers to a dose that is adequate to prevent or treat a neoplastic disease, e.g., a cancer, in an individual. Amounts effective for a therapeutic or prophylactic use will depend on, for example, the stage and severity of the disease or disorder being treated, the age, weight, and general state of health of the patient, and the judgment of the prescribing physician. The size of the dose will also be determined by the active selected, method of administration, timing and frequency of administration, the existence, nature, and extent of any adverse side-effects that might accompany the administration of a particular active, and the desired physiological effect. It will be appreciated by one of skill in the art that various diseases or disorders could require prolonged treatment involving multiple administrations, perhaps using the inventive CAR construct materials in each or various rounds of administration. By way of example and not intending to limit the invention, when the inventive CAR construct material is a host cell, an exemplary dose of host cells may be a minimum of one million cells (1×10⁶ cells/dose).

For purposes of the invention, the amount or dose of an agent comprising an immune cell containing a CAR construct described herein administered should be sufficient to effect a therapeutic or prophylactic response in the subject or animal over a reasonable time frame. For example, the dose should be sufficient to bind to antigen, or detect, treat or prevent cancer in a period of from about 2 hours or longer, e.g., about 12 to about 24 or more hours, from the time of administration. In certain embodiments, the time period could be even longer. The dose will be determined by the efficacy of the particular inventive CAR construct material and the condition of the animal (e.g., human), as well as the body weight of the animal (e.g., human) to be treated.

Effector function: As used herein, “effector function” or “effector activity” refers to a specific activity carried out by an immune cell in response to stimulation of the immune cell. For example, an effector function of a T lymphocyte includes, recognizing an antigen and killing a cell that expresses the antigen.

Encoding: As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Endogenous: As used herein “endogenous” refers to any material from or produced inside a particular organism, cell, tissue or system.

Exogenous: As used herein, the term “exogenous” refers to any material introduced from or produced outside a particular organism, cell, tissue or system.

Expand: As used herein, the term “expand” refers to increasing in number, as in an increase in the number of cells, for example, immune cells, e.g., T lymphocytes, and/or hematopoietic cells. In one embodiment, immune cells, e.g., T lymphocytes, and/or hematopoietic cells that are expanded ex vivo increase in number relative to the number originally present in a culture. In another embodiment, immune cells, e.g., T lymphocytes, and/or hematopoietic cells that are expanded ex vivo increase in number relative to other cell types in a culture. In some embodiments, expansion may occur in vivo. The term “ex vivo,” as used herein, refers to cells that have been removed from a living organism, (e.g., a human) and propagated outside the organism (e.g., in a culture dish, test tube, or bioreactor).

Expression: As used herein, the term “expression” of a nucleic acid sequence refers to generation of any gene product from a nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Expression vector. As used herein, the term “expression vector” or “recombinant expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses).

Fragment: As used herein, the terms “fragment” or “portion” refers to a structure that includes a discrete portion of the whole but lacks one or more moieties found in the whole structure. In some embodiments, a fragment consists of such a discrete portion. In some embodiments, a fragment consists of or comprises a characteristic structural element or moiety found in the whole. In some embodiments, a nucleotide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500, or more monomeric units (e.g., nucleic acids) as found in the whole nucleotide. In some embodiments, a nucleotide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99%, or more of the monomeric units (e.g., residues) found in the whole nucleotide. The whole material or entity may in some embodiments be referred to as the “parent” of the whole.

Functional Portion: As used herein, the term “functional portion” when used in reference to a CAR refers to any part or fragment of the CAR constructs of the invention, which part or fragment retains the biological activity of the CAR construct of which it is a part (the parent CAR construct). Functional portions encompass, for example, those parts of a CAR construct that retain the ability to recognize target cells, or detect, treat, or prevent cancer, to a similar extent, the same extent, or to a higher extent, as the parent CAR construct. In reference to the parent CAR construct, the functional portion can comprise, for instance, about 10%, about 25%, about 30%, about 50%, about 68%, about 80%, about 90%, about 95%, or more, of the parent CAR.

The functional portion can comprise additional amino acids at the amino or carboxy terminus of the portion, or at both termini, which additional amino acids are not found in the amino acid sequence of the parent CAR construct. Desirably, the additional amino acids do not interfere with the biological function of the functional portion, e.g., recognize target cells, detect cancer, treat or prevent cancer, etc. More desirably, the additional amino acids enhance the biological activity as compared to the biological activity of the parent CAR construct.

Functional Variant: As used herein, the term “functional variant,” as used herein, refers to a CAR construct, polypeptide, or protein having substantial or significant sequence identity or similarity to a parent CAR construct, which functional variant retains the biological activity of the CAR of which it is a variant. Functional variants encompass, for example, those variants of the CAR construct described herein (the parent CAR construct) that retain the ability to recognize target cells to a similar extent, the same extent, or to a higher extent, as the parent CAR construct. In reference to the parent CAR construct, the functional variant can, for instance, be at least about 30%, about 50%, about 75%, about 80%, about 90%, about 91%, about 92%, about 93%, about 94%, about 95%, about 96%, about 97%, about 98%, about 99% or more identical in amino acid sequence to the parent CAR construct. A functional variant can, for example, comprise the amino acid sequence of the parent CAR with at least one conservative amino acid substitution. Alternatively or additionally, the functional variants can comprise the amino acid sequence of the parent CAR construct with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR construct.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences.

Identity: As used herein, the term “identity” refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

Substantial identity: As used herein, the term “substantial identity” refers to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues. In the context of a CDR, reference to “substantial identity” typically refers to a CDR having an amino acid sequence at least 80%, preferably at least 85%, at least 90%, at least 95%, at least 98% or at least 99% identical to that of a reference CDR.

Immune cell: As used herein, the term “immune cell,” used interchangeably herein with the term “immune effector cell,” refers to a cell that is involved in an immune response, e.g., promotion of an immune response. Examples of immune cells include, but are not limited to, T-lymphocytes, natural killer (NK) cells, macrophages, monocytes, dendritic cells, neutrophils, eosinophils, mast cells, platelets, large granular lymphocytes, Langerhans' cells, or B-lymphocytes. A source of immune cells (e.g., T lymphocytes) can be obtained from a subject.

Immune response: As used herein the term “immune response” refers to a cellular and/or systemic response to an antigen that occurs when lymphocytes identify antigenic molecules as foreign and induce the formation of antibodies and/or activate lymphocytes to remove the antigen.

Immunoglobulin: As used herein, the term “immunoglobulin” or “Ig,” refers to a class of proteins that function as antibodies. Antibodies expressed by B cells are sometimes referred to as a BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is an immunoglobulin that has no known antibody function but may serve as an antigen receptor. IgE is an immunoglobulin that mediates immediate hypersensitivity by causing release of mediators from mast cells and basophils upon exposure to allergen.

Isolated: As used herein, the term “isolated” refers to something altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

Modified: As used herein, the term “modified” refers to a changed state or structure of a molecule or cell of the invention. Molecules may be modified in many ways, including chemically, structurally, and functionally. Cells may be modified through the introduction of nucleic acids.

Modulating: As used herein the term “modulating,” refers to mediating a detectable increase or decrease in the level of a response and/or a change in the nature of a response in a subject compared with the level and/or nature of a response in the subject in the absence of a treatment or compound, and/or compared with the level and/or nature of a response in an otherwise identical but untreated subject. The term encompasses perturbing and/or affecting a native signal or response thereby mediating a beneficial therapeutic response in a subject, preferably, a human.

Monoclonal Antibody: A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing naturally occurring mutations or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

Nucleic acid: As used herein, the term “nucleic acid” refers to a polymer of at least three nucleotides. In some embodiments, a nucleic acid comprises DNA. In some embodiments, a nucleic acid comprises RNA. In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises one or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long.

Operably linked: As used herein, the term “operably linked,” used herein in the context of nucleic acid constructs, and used interchangeably with the term “under the control of” refers to functional linkage between, for example, a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. In such embodiments, expression of the coding sequence is “under the control of” the promoter. Operably linked DNA sequences are typically contiguous.

Polynucleotide: As used herein, the term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

Polypeptide: As used herein, the term “polypeptide” refers to any polymeric chain of residues (e.g., amino acids) that are typically linked by peptide bonds. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence) or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Signal transduction pathway: As used herein, the term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

Single chain antibodies: As used herein, the term “single chain antibodies” refers to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

Specifically binds: As used herein, the term “specifically binds,” with respect to an antigen binding domain, such as an antibody agent, refers to an antigen binding domain or antibody agent which recognizes a specific antigen, but does not substantially recognize or bind other molecules in a sample. For example, an antigen binding domain or antibody agent that specifically binds to an antigen from one species may also bind to that antigen from one or more species. But, such cross-species reactivity does not itself alter the classification of an antigen binding domain or antibody agent as specific. In another example, an antigen binding domain or antibody agent that specifically binds to an antigen may also bind to different allelic forms of the antigen. However, such cross reactivity does not itself alter the classification of an antigen-binding domain or antibody agent as specific. In some instances, the terms “specific binding” or “specifically binding,” can be used in reference to the interaction of an antigen binding domain or antibody agent, a protein, or a peptide with a second chemical species, to mean that the interaction is dependent upon the presence of a particular structure (e.g., an antigenic determinant or epitope) on the chemical species; for example, an antigen binding domain or antibody agent recognizes and binds to a specific protein structure rather than to proteins generally. If an antigen binding domain or antibody agent is specific for epitope “A”, the presence of a molecule containing epitope A (or free, unlabeled A), in a reaction containing labeled “A” and the antigen binding domain or antibody agent, will reduce the amount of labeled A bound to the antibody.

Subject: As used herein, the term “subject” refers to an organism, for example, a mammal (e.g., a human, a non-human mammal, a non-human primate, a primate, a laboratory animal, a mouse, a rat, a hamster, a gerbil, a cat, or a dog). In some embodiments, a human subject is an adult, adolescent, or pediatric subject. In some embodiments, a subject is suffering from a disease, disorder or condition, e.g., a disease, disorder, or condition that can be treated as provided herein, e.g., a cancer or a tumor listed herein. In some embodiments, a subject is susceptible to a disease, disorder, or condition; in some embodiments, a susceptible subject is predisposed to and/or shows an increased risk (as compared to the average risk observed in a reference subject or population) of developing the disease, disorder, or condition. In some embodiments, a subject displays one or more symptoms of a disease, disorder, or condition. In some embodiments, a subject does not display a particular symptom (e.g., clinical manifestation of disease) or characteristic of a disease, disorder, or condition. In some embodiments, a subject does not display any symptom or characteristic of a disease, disorder, or condition. In some embodiments, a subject is a patient. In some embodiments, a subject is an individual to whom diagnosis and/or therapy is and/or has been administered.

Substantially purified: As used herein, the term “substantially purified,” for example as applied to a cell, refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

Target: As used herein, the term “target” refers to a cell, tissue, organ, or site within the body that is the subject of provided methods, systems, and/or compositions, for example, a cell, tissue, organ or site within a body that is in need of treatment or is preferentially bound by, for example, an antibody (or fragment thereof) or a CAR.

Target site: As used herein, the term “target site” or “target sequence” refers to a genomic nucleic acid sequence that defines a portion of a nucleic acid to which a binding molecule (e.g., an antigen-binding domain of a CAR) may specifically bind under conditions sufficient for binding to occur.

T cell receptor: As used herein, the term “T cell receptor” or “TCR” refers to a complex of membrane proteins that participate in the activation of T cells in response to the presentation of antigen. A TCR is responsible for recognizing antigens bound to major histocompatibility complex molecules. A TCR comprises a heterodimer of an alpha (α) and beta (β) chain, although in some cells the TCR comprises gamma and delta (γ/δ) chains. TCRs may exist in alpha/beta and gamma/delta forms, which are structurally similar but have distinct anatomical locations and functions. Each chain comprises two extracellular domains, a variable and constant domain. In some embodiments, a TCR may be modified on any cell comprising a TCR, including, for example, a helper T cell, a cytotoxic T cell, a memory T cell, regulatory T cell, natural killer T cell, and gamma delta T cell.

Therapeutic: As used herein, the term “therapeutic” refers to a treatment and/or prophylaxis. A therapeutic effect is obtained by suppression, remission, or eradication of a disease state.

Transfected: As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

Transgene: As used herein, the term “transgene” refers to an exogenous nucleic acid sequence comprised in a cell, e.g., in the genome of the cell, in which the nucleic acid sequence does not naturally occur. In some embodiments, a transgene may comprise or consist of a nucleic acid sequence encoding a gene product, e.g., a CAR. In some embodiments, a transgene may comprise or consist of an expression construct, e.g., a nucleic acid sequence encoding a gene product under the control of a regulatory element, e.g., a promoter.

Treat: As used herein, the term “treat,” “treatment,” or “treating” refers to partial or complete alleviation, amelioration, delay of onset of, inhibition, prevention, relief, and/or reduction in incidence and/or severity of one or more symptoms or features of a disease, disorder, and/or condition. In some embodiments, treatment may be administered to a subject who does not exhibit signs or features of a disease, disorder, and/or condition (e.g., may be prophylactic). In some embodiments, treatment may be administered to a subject who exhibits only early or mild signs or features of the disease, disorder, and/or condition, for example for the purpose of decreasing the risk of developing pathology associated with the disease, disorder, and/or condition. In some embodiments, treatment may be administered to a subject who exhibits established, severe, and/or late-stage signs of the disease, disorder, or condition. In some embodiments, treating may comprise administering to an immune cell (e.g., a T lymphocyte) or contacting an immune cell with a modulator of a pathway activated by in vitro transcribed mRNA.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

Vector: As used herein, the term “vector” refers to a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

### DETAILED DESCRIPTION

While CAR-based immunotherapies have proven to be effective in the clinic, e.g., in the context of treating certain cancers, it remains challenging to control expression of the CAR constructs in immunotherapeutic effector cells, such as, for example, T-cells or natural killer (NK) cells. Conventional expression systems typically employ promoters that are constitutively active and broadly expressed amongst different cell types, such as, for example, the eukaryotic translation elongation factor 1 alpha (EF1a) promoter, to drive expression of CAR constructs in effector cells, such as T-cells or NK cells.

Some aspects of this disclosure provide expression systems that are useful for specifically expressing a CAR in an immunotherapeutic cell type, e.g., a CAR-T or CAR-NK cell. In some embodiments, the expression system comprises a regulatory element, e.g., a promoter, that is specifically active only in the immunotherapeutic cell type (e.g., in T cell or NK cells), or in a cell lineage that the immunotherapeutic cell type belongs to (e.g., in lymphocytes). Some aspects of this disclosure provide expression systems that are useful for specifically expressing a CAR in an immunotherapeutic cell type, e.g., a CAR-T or CAR-NK cell, from a non-constitutive promoter, for example, from an inducible or repressible promoter. Examples of inducible and repressible expression systems include, for example, tetracycline-controlled expression systems, like the tet-on expression systems.

Some aspects of this disclosure provide a CAR expression system comprising (i) a first expression cassette comprising a nucleic acid encoding a tetracycline-responsive transactivator under the control of a cell type-specific promoter, e.g., a cell type-specific constitutive promoter, and (ii) a second expression cassette comprising a nucleic acid encoding a CAR under the control of a tetracycline-responsive promoter.

The cell type-specific and inducible CAR expression systems provided herein are useful, e.g., for the generation of CAR-expressing immunotherapeutic effector cells (e.g., CAR-T or CAR-NK cells) from hematopoietic stem cells or hematopoietic progenitor cells (HSCs or HPCs, respectively) comprising such a CAR expression system, e.g., via in vitro differentiation or in vivo via population of a stem- or progenitor cell niche and subsequent in vivo differentiation, where CAR expression is not desired or detrimental in cells other than the immunotherapy effector cells. For example, where CAR-T cells are derived in vitro from CD34+ HSCs, it may be advantageous to restrict CAR expression to later stages of hematopoietic lineage differentiation, e.g., to pre-T and T cells, instead of expressing the CAR more broadly across hematopoietic cell lineages, or in all stages of differentiation. For another example, where CAR-T cells are derived in vivo in a subject, e.g. after transfusion of CD34+ HSCs, it may be advantageous to restrict CAR expression to later stages of hematopoietic lineage differentiation, e.g., to pre-T and T cells, instead of expressing the CAR more broadly across hematopoietic cell lineages, or in all stages of differentiation.

The cell type-specific and inducible CAR expression systems provided herein are also useful, e.g., for the temporal control of CAR-expression in immunotherapeutic effector cells (e.g., CAR-T or CAR-NK cells), whether they are derived from HSCs or HPCs, or directly from immune effector cell preparations, e.g., from T-cell or NK cell preparations isolated from a subject.

### Expression Constructs

Some aspects of this disclosure provide expression constructs useful for expressing a transgene in a specific cell type in an inducible manner. For example, some aspects of this disclosure provide expression constructs that are only active in specific cell types, such as, for example, hematopoietic cells, or specific cell types or sub-types of the hematopoietic cell lineage (e.g., T-lymphocytes, pre-T cells, mature T-lymphocytes, NK cells, etc.). In some embodiments, cell type specific and inducible expression is achieved by expressing an inducible expression system, e.g., a tetracycline-inducible system, in a cell type-specific manner, e.g., by placing one of the components of the inducible system under the control of a regulatory element, e.g., a promoter, that is only active in the respective cell type.

For example, in some embodiments, expression systems are provided herein in which a tet-on transactivator (rTTA) is operably linked to a cell-type specific promoter. The rTTA is thus only expressed in cells in which the promoter is active. For example, if the cell-type specific promoter is a T-cell specific promoter, e.g., a CD8 or TCRb promoter, the rTTA is only expressed in T cells or pre-T-cells, but not in other cells of the hematopoietic lineage, or in any other cell types.

The inducible expression strategies can be used with any suitable expression constructs, e.g., plasmid-based expression constructs, linear DNA expression constructs, viral expression construct (e.g., retroviral expression constructs, lentiviral expression constructs, adenoviral expression constructs, and AAV-based expression constructs, etc.), transposase-based expression constructs. Additional suitable expression constructs useful for the creation of a cell-type inducible expression system as provided herein will be apparent to the skilled artisan based on the present disclosure. The disclosure is not limited in this respect.

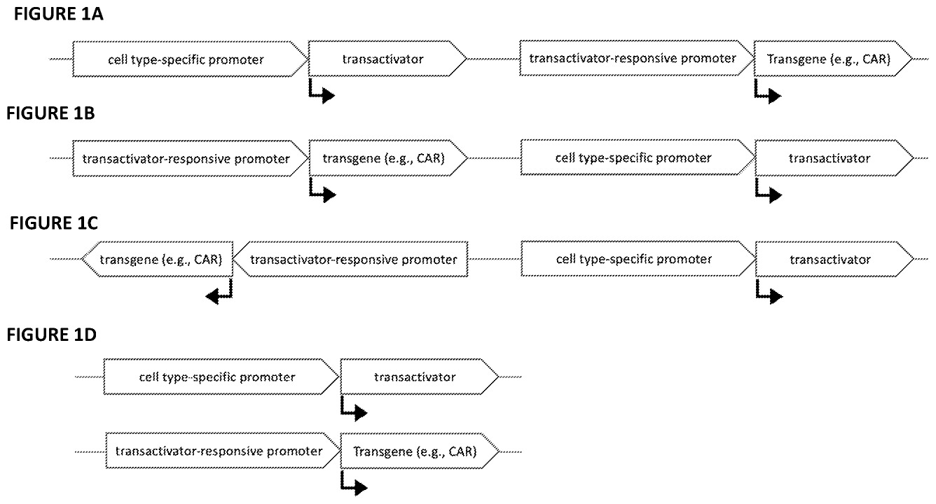

Some aspects of this disclosure provide nucleic acid constructs comprising a first expression cassette comprising a nucleic acid encoding a tetracycline-responsive transactivator under the control of a cell type-specific promoter; and a second expression cassette comprising a nucleic acid encoding a transgene. In some embodiments, an expression construct is provided that comprises both expression cassettes on the same DNA molecule, e.g., on the same double-stranded DNA molecule, or on the same single-stranded DNA molecule. Various configurations of the expression cassettes can be used, e.g., as illustrated in FIGS. 1A-1D. For example, both expression cassettes can be oriented in the same direction. In some embodiments, the first expression cassette is 5′ (upstream) of the second expression cassette. In some embodiments, the first expression cassette is 3′ (downstream) of the second expression cassette. The expression cassettes can also be oriented in different directions. In some embodiments, the expression cassettes are oriented in a manner in which the promoter of the first expression cassette points toward the second expression cassette, and the promoter of the second expression cassette points towards the first expression cassette. In some embodiments, the expression cassettes are oriented in a manner in which the promoter of the first expression cassette points away from the second expression cassette, and the promoter of the second expression cassette points away from first expression cassette. One advantage of this latter configuration is that transcriptional interference between the two expression cassettes is avoided or minimized. It also allows for the use of a unidirectional polyadenylation signal in viral expression vectors for the expression cassette that is oriented towards the 5′ end of the viral backbone (e.g., the 5′ LTR in retroviral vectors), for example, where the use of a polyadenylation signal oriented in the same direction as viral RNA transcription might interfere with the generation of full-length viral RNA and thus hinder the generation of full-length viral particles.

In some embodiments, the cell-type specific promoter is a promoter that is expressed in one specific cell type, e.g., in T-lymphocytes, B-lymphocytes, NK cells, or in a specific sub-group of cells, e.g., in lymphocytes, but not in cells other than the specific cell type. For example, a cell-type specific promoter may be a promoter expressed in certain cells of the hematopoietic lineage, e.g., in T-lymphocytes or NK cells, but not in hematopoietic stem cells, or in other cells of the hematopoietic lineage.

Exemplary cell-type specific promoters are provided herein. For example, in some embodiments, expression constructs are provided in which a T-cell specific promoter is used. In some embodiments, the T-cell specific promoter is a CD4 promoter. In some embodiments, the T-cell specific promoter is a CD8a promoter. In some embodiments, the T-cell specific promoter is a CD8b promoter. In some embodiments, the T-cell specific promoter is a TCRa promoter. In some embodiments, the T-cell specific promoter is a TCRb promoter. In some embodiments, the T-cell specific promoter is a CD3d promoter (CD3d, CD3D, and CD3delta, are used interchangeably herein). In some embodiments, the T-cell specific promoter is a CD3g promoter. In some embodiments, the T-cell specific promoter is a CD3e promoter (CD3e, CD3E, and CD3epsilon are used interchangeably herein). In some embodiments, the T-cell specific promoter is a CD3z promoter. In some embodiments, the T-cell specific promoter is a actin promoter. In some embodiments, the T-cell specific promoter is a CD25 promoter. In some embodiments, the T-cell specific promoter is an IL2 promoter. In some embodiments, the T-cell specific promoter is a CD69 promoter. In some embodiments, the T-cell specific promoter is a GzmB promoter. In some embodiments, the T-cell specific promoter is a T-bet promoter. In some embodiments, the T-cell specific promoter is an IFNgamma promoter. In some embodiments, the T-cell specific promoter is a TIM3 promoter. In some embodiments, the T-cell specific promoter is a IL4 promoter. In some embodiments, the T-cell specific promoter is a GATA3 promoter. In some embodiments, the T-cell specific promoter is an IL5 promoter. In some embodiments, the T-cell specific promoter is an IL13 promoter. In some embodiments, the T-cell specific promoter is an IL10 promoter. In some embodiments, the T-cell specific promoter is an IL17A promoter. In some embodiments, the T-cell specific promoter is an IL6 promoter. In some embodiments, the T-cell specific promoter is an IL21 promoter. In some embodiments, the T-cell specific promoter is an IL23R promoter. In some embodiments, the T-cell specific promoter is a FoxP3 promoter. In some embodiments, the T-cell specific promoter is a CTLA4 promoter. In some embodiments, the T-cell specific promoter is a CD25 promoter. In some embodiments, the T-cell specific promoter is a PD1 promoter. In some embodiments, the T-cell specific promoter is a CD45RO promoter. In some embodiments, the T-cell specific promoter is a CCR7 promoter. In some embodiments, the T-cell specific promoter is a CD28 promoter. In some embodiments, the T-cell specific promoter is a CD95 promoter. In some embodiments, the T-cell specific promoter is a CD28 promoter. In some embodiments, the T-cell specific promoter is a CD27 promoter. In some embodiments, the T-cell specific promoter is a CD127 promoter. In some embodiments, the T-cell specific promoter is a PD-1 promoter. In some embodiments, the T-cell specific promoter is a CD122 promoter. In some embodiments, the T-cell specific promoter is a CD132 promoter. In some embodiments, the T-cell specific promoter is a KLRG-1 promoter. In some embodiments, the T-cell specific promoter is an HLA-DR promoter. In some embodiments, the T-cell specific promoter is a CD38 promoter. In some embodiments, the T-cell specific promoter is a CD69 promoter. In some embodiments, the T-cell specific promoter is a Ki-67 promoter. In some embodiments, the T-cell specific promoter is a CD11a promoter. In some embodiments, the T-cell specific promoter is a CD58 promoter. In some embodiments, the T-cell specific promoter is a CD99 promoter. In some embodiments, the T-cell specific promoter is a CD62L promoter. In some embodiments, the T-cell specific promoter is a CD103 promoter. In some embodiments, the T-cell specific promoter is a CCR4 promoter. In some embodiments, the T-cell specific promoter is a CCR5 promoter. In some embodiments, the T-cell specific promoter is a CCR6 promoter. In some embodiments, the T-cell specific promoter is a CCR9 promoter. In some embodiments, the T-cell specific promoter is a CCR10 promoter. In some embodiments, the T-cell specific promoter is a CXCR3 promoter. In some embodiments, the T-cell specific promoter is a CXCR4 promoter. In some embodiments, the T-cell specific promoter is a CLA promoter. In some embodiments, the T-cell specific promoter is a Granzyme A promoter. In some embodiments, the T-cell specific promoter is a Granzyme B promoter. In some embodiments, the T-cell specific promoter is a Perforin promoter. In some embodiments, the T-cell specific promoter is a CD57 promoter. In some embodiments, the T-cell specific promoter is a CD161 promoter. In some embodiments, the T-cell specific promoter is an IL-18Ra promoter. In some embodiments, the T-cell specific promoter is a c-Kit promoter. In some embodiments, the T-cell specific promoter is a CD130 promoter.

In some embodiments, the T-cell specific promoter is a CD4 promoter, a CD8a promoter, a CD8b promoter, a TCRa promoter, a TCRb promoter, a CD3d promoter, a CD3g promoter, a CD3e promoter, or a CD3z promoter.

In some embodiments, the cell-type specific promoter is an NK cell specific promoter. In some embodiments, the NK cell specific promoter is a KLRK1 promoter. In some embodiments, the T-cell specific promoter is a CD56 promoter. In some embodiments, the T-cell specific promoter is a CD94 promoter. In some embodiments, the T-cell specific promoter is a CD122 promoter. In some embodiments, the T-cell specific promoter is a CD127 promoter. In some embodiments, the T-cell specific promoter is an IL-2Ralpha promoter. In some embodiments, the T-cell specific promoter is a Fc gamma RIII promoter. In some embodiments, the T-cell specific promoter is a CD16 promoter. In some embodiments, the T-cell specific promoter is a KIR family receptor promoter. In some embodiments, the T-cell specific promoter is a NKG2A promoter. In some embodiments, the T-cell specific promoter is a NKG2D promoter. In some embodiments, the T-cell specific promoter is a NKp30 promoter. In some embodiments, the T-cell specific promoter is a NKp44 promoter. In some embodiments, the T-cell specific promoter is a NKp46 promoter. In some embodiments, the T-cell specific promoter is a NKp80 promoter.

While some exemplary cell-type specific promoters are provided herein, additional suitable cell-type specific promoters will be apparent to those of skill in the art based on the present disclosure and in view of the knowledge in the art. The present disclosure is not limited in this respect.

Some aspects of this disclosure provide nucleic acid constructs comprising a first expression cassette comprising a nucleic acid encoding a tetracycline-responsive transactivator under the control of a cell type-specific promoter; and a second expression cassette comprising a nucleic acid encoding a transgene, e.g., a chimeric antigen receptor, under the control of a tetracycline-responsive promoter. In some embodiments, the transgene encodes a chimeric antigen receptor targeting a human antigen associated with a disease or disorder. In some embodiments, the cell type-specific promoter is a constitutive promoter. In some embodiments, the cell type specific promoter is a CD8 promoter. In some embodiments, the cells type-specific promoter is a CD3delta promoter. In some embodiments, the cells type-specific promoter is a CD3epsilon promoter. In some embodiments, the cell type-specific promoter is a CD56 promoter. In some embodiments, the cell type-specific promoter is a CD244 promoter. In some embodiments, the cell type-specific promoter is a CD94 promoter. In some embodiments, the cell type-specific promoter is an NKG2D promoter.

The expression constructs of the present disclosure feature a tetracycline-responsive expression system, e.g., a tet-on expression system. Exemplary tet-on expression systems are described herein. Additional suitable tet-on expression systems for use in connection with the inventive concepts provided herein will be apparent to the skilled artisan based on the present disclosure and the knowledge of tet-on expression systems in the art, as illustrated, for example, in the following, non-limiting, examples of publications: Gossen et al., “Transcriptional activation by tetracyclines in mammalian cells”. Science. 268 (5218):1766-9, 1995; Orth et al., “Structural basis of gene regulation by the tetracycline inducible Tet repressor-operator system”. Nature Structural & Molecular Biology. 7 (3): 215-219, 2000; Gossen et al., “Tight control of gene expression in mammalian cells by tetracycline-responsive promoters”. Proc. Natl. Acad. Sci. U.S.A. 89 (12): 5547-51, 1992; Urlinger et al., “Exploring the sequence space for tetracycline-dependent transcriptional activators: novel mutations yield expanded range and sensitivity”. Proc. Natl. Acad. Sci. U.S.A. 97 (14): 7963-8, 2000; Das et al., “Tet-On Systems for Doxycycline-inducible Gene Expression”. Current Gene Therapy. 16 (3): 156-67, 2016; and Zhou et al., “Optimization of the Tet-On system for regulated gene expression through viral evolution”. Gene Ther. 13 (19): 1382-90, 2006.

In some embodiments, the tetracycline-responsive transactivator is an rtTA protein. In some embodiments, the tetracycline-responsive transactivator is an rtTA2^{S}-M2 protein. In some embodiments, the tetracycline-responsive transactivator is an rtTA-V10 protein. In some embodiments, the tetracycline-responsive transactivator is an rtTA-V16 protein. In some embodiments, the tetracycline-responsive transactivator is an rtTA-3G protein. Tetracycline (or doxycycline)-inducible expression systems, including tet-on and tet-off systems, and their elements, e.g., transactivators, tet response elements, tetracycline responsive operators and promoters, as well as suitable tetracycline formulations, and formulations of tetracycline analogs, such as doxycycline, are well known to the skilled artisan. Some exemplary embodiments, using rtTA-3G transactivators, are provided herein. Other suitable tetracycline inducible systems will be apparent to those of skill in the art based on the present disclosure. The disclosure is not limited in this respect.

In some embodiments, the transgene encodes a chimeric antigen receptor (CAR) comprising a binding domain, a hinge domain, a transmembrane domain, at least one co-stimulatory domain, a cytoplasmic signaling domain, or a combination thereof. In some embodiments, the binding domain comprises an antibody, or an antigen-binding antibody fragment, that binds an antigen. In some embodiments, the binding domain comprises an scFv or a single domain antibody that binds to an antigen. In some embodiments, the antigen is a lineage-specific cell-surface antigen. In some embodiments, expression of the antigen is associated with a neoplastic or malignant disease. In some embodiments, the antigen is CD33, CD123, CD19, or CLL-1. In some embodiments, the hinge domain of the CAR is a CD8a (CD8alpha) hinge domain. In some embodiments, the transmembrane domain of the CAR is a CD8 or CD28 transmembrane domain. In some embodiments, the costimulatory domain of the CAR is a 4-1BB or CD28 costimulatory domain, or a combination thereof. In some embodiments, the cytoplasmic signaling domain of the CAR is a CD3 (CD3zeta) cytoplasmic signaling domain.

In some embodiments, the CAR comprises an antigen binding domain that binds to an antigen that is associated with a disease or disorder, e.g., with a neoplastic or malignant disease or disorder.

In some embodiments, the CAR is a first generation CAR. In some embodiments, the CAR is a second generation CAR. In some embodiments, the CAR is a third generation CAR. In some embodiments, the CAR is a fourth or fifth generation CAR, or an armored CAR. Exemplary CAR constructs are provided herein, and additional suitable CAR constructs will be apparent to the skilled artisan based on the present disclosure and the knowledge in the art. For an illustration of various CAR backbones or frameworks that are suitable for use in connection with the presently provided expression systems, see, the following, exemplary, and non-limiting publications: Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

First generation CARs are typically composed of an extracellular antigen binding domain, for example, a single-chain variable fragment (scFv), fused to a transmembrane domain, which is fused to a cytoplasmic/intracellular domain of the T cell receptor chain. Typically, first generation CARs comprise the intracellular domain of CD3ζ, which transmits signals from endogenous T cell receptors (TCRs) “First generation” CARs can provide de novo antigen recognition and cause activation of both CD4⁺ and CD8⁺ T cells through their CD3ζ chain signaling domain in a single fusion molecule, independent of HLA-mediated antigen presentation.

Second-generation CARs comprise an antigen-binding domain fused to an intracellular signaling domain capable of activating T cells and a co-stimulatory domain designed to augment T cell potency and persistence. See, e.g., Sadelain et al., Cancer Discov. 3:388-398, 2013. Second generation CARs comprise an intracellular co-stimulatory domain in addition to the CD3ζ domain, for example, a CD28, 4-1BB, ICOS, OX40, or similar co-stimulatory domain. Thus, second generation CARs provide both co-stimulation, for example, by CD28 or 4-1BB domains, and activation, for example, by a CD3ζ signaling domain. Second Generation CARs may improve the anti-tumor activity of T cells as compared to first generation CARs.

Third generation CARs comprise more than one co-stimulatory domains, for example, two costimulatory domains, e.g., both a CD28 and a 4-1BB domain, and an activation domain, for example, by comprising a CD3ζ activation domain.

In general, CARs comprise an extracellular antigen binding domain, a transmembrane domain and an intracellular domain. Typically, the antigen binding domain binds to an antigen of interest, such as an antigen associated with a disease or disorder, e.g., with a neoplastic or malignant disease or disorder. In some embodiments, the antigen-binding domain is an antibody or an antigen-binding fragment thereof. In some embodiments, the antigen binding domain is a single chain antibody or antigen-binding fragment thereof. In some embodiments, the antigen-binding domain comprises an scFv. In some embodiments, the antigen-binding domain comprises a single domain antibody, e.g., a camelid antibody, or a humanized derivative thereof. In some embodiments, the antigen-binding domain comprises a receptor or a receptor ligand.

Some exemplary CARs for use in the expression systems disclosed herein are provided herein. Additional CARs will be apparent to the skilled artisan based on the present disclosure in view of the knowledge in the art regarding the design of CARs, e.g., as illustrated in Sadelain et al., Cancer Discov. 3(4):388-398 (2013); Jensen et al., Immunol. Rev. 257:127-133 (2014); Sharpe et al., Dis. Model Mech. 8(4):337-350 (2015), and references cited therein).

In some embodiments, a CAR is used in connection with the cell-type specific inducible expression systems disclosed herein that comprises an scFv or single domain antibody or antigen-binding fragment thereof, to a CAR backbone, e.g., a first generation, second generation, or third or later generation, CAR backbone.

In some embodiments, the CAR for use in the present invention comprises an extracellular domain that includes an antigen binding domain that binds to an antigen associated with a disease or disorder, e.g., associated with a neoplastic or malignant disease or disorder. In some embodiments, the antigen binding domain binds to an antigen expressed on the surface of a neoplastic cell or a malignant cell. In some embodiments, the antigen binding domain binds to an antigen expressed or present on the surface of a pathogenic or pathologic cell, e.g., a cell that has been infected by an infectious agent, or a cell that is characterized by a pathogenic or pathologic state. In some embodiments, the antigen binding domain can be an scFv or a Fab, a single domain antibody, or any suitable antigen binding fragment of an antibody (see Sadelain et al., Cancer Discov. 3:38-398 (2013)).

In some embodiments, the antigen binding domain comprises a sequence of a human, a humanized, a chimeric, or a CDR-grafted antibody, or antigen-binding antibody fragment. In some embodiments, the antigen binding domain comprises an scFv. Exemplary scFvs are provided herein, and additional suitable scFvs will be apparent to the skilled artisan based on the present disclosure and the knowledge in the art related to scFvs (see, for example, Huston, et al., Proc. Nat. Acad. Sci. USA 85:5879-5883 (1988); Ahmad et al., Clin. Dev. Immunol. 2012: ID980250 (2012); U.S. Pat. Nos. 5,091,513, 5,132,405 and 4,956,778; and U.S. Patent Publication Nos. 20050196754 and 20050196754)). The disclosure is not limited in this aspect.

Alternatively to using an antigen binding domain derived from an antibody, a CAR extracellular domain can comprise a ligand or extracellular ligand binding domain of a receptor (see Sadelain et al., Cancer Discov. 3:388-398 (2013); Sharpe et al., Dis. Model Mech. 8:337-350 (2015)).

In some embodiments, a CAR binds to a target antigen that is expressed on the surface of a target cell, e.g., a neoplastic or malignant cell. In some embodiments, the target antigen is specifically expressed on a target cell, but not on non-pathogenic or non-pathologic cells, e.g., in a subject. In some embodiments, the target antigen is overexpressed on the surface of a target cell, e.g., a neoplastic or malignant cell, while non-pathologic or non-pathogenic cells express the target antigen at significantly lower levels, e.g., at a level of less than 10%, less than 1%, less than 0.1%, less than 0.01%, or less than 0.001% of the level of expression in the pathogenic or pathologic cell, e.g., the neoplastic or malignant cell. In some embodiments, the target antigen to be bound by the CAR is chosen to provide targeting of cells expressing the antigen, e.g., neoplastic or malignant cells, over non-target cells, e.g., healthy, non-neoplastic or non-malignant cells.

In some embodiments, the CAR binds to an antigen expressed on malignant cells, which is also referred to sometimes as a cancer antigen. Any CAR targeting a suitable cancer antigen can be used in the context of the presently disclosed expression systems. Exemplary cancer antigens and exemplary cancers are provided below.

In some embodiments, a CAR expressed using a cell-type specific, inducible expression system as disclosed herein binds to a cancer antigen listed below: CD5, CD6, CD7, CD10, CD19, CD20, CD22, CD30, CD33, CD34, CD38, CD41, CD44, CD49f, CD56, CD74, CD123, CD133, CD138, mesothelin (MSLN), prostate specific membrane antigen (PSMA), prostate stem cell antigen (PCSA), carbonic anhydrase IX (CAIX), carcinoembryonic antigen (CEA), epithelial glycoprotein2 (EGP 2), epithelial glycoprotein-40 (EGP-40), epithelial cell adhesion molecule (EpCAM), folate-binding protein (FBP), fetal acetylcholine receptor (AChR), folate receptor-α and β (FRα and β), Ganglioside G2 (GD2), Ganglioside G3 (GD3), human Epidermal Growth Factor Receptor 2 (HER-2/ERB2), Epidermal Growth Factor Receptor vIII (EGFRvIII), ERB3, ERB4, human telomerase reverse transcriptase (hTERT), Interleukin-13 receptor subunit alpha-2 (IL-13Rα2), κ-light chain, kinase insert domain receptor (KDR), Lewis A (CA19.9), Lewis Y (LeY), L1 cell adhesion molecule (L1CAM), melanoma-associated antigen 1 (melanoma antigen family A1, MAGE-A1), Mucin 16 (Muc-16), Mucin 1 (Muc-1), NKG2D ligands, cancer-testis antigen NY-ESO-1, oncofetal antigen (h5T4), tumor-associated glycoprotein 72 (TAG-72), vascular endothelial growth factor R2 (VEGF-R2), Wilms tumor protein (WT-1), type 1 tyrosine-protein kinase transmembrane receptor (ROR1), B7-H3 (CD276), B7-H6 (Nkp30), Chondroitin sulfate proteoglycan-4 (CSPG4), DNAX Accessory Molecule (DNAM-1), Ephrin type A Receptor 2 (EpHA2), Fibroblast Associated Protein (FAP), Gp100/HLA-A2, Glypican 3 (GPC3), HA-1H, HERK-V, IL-11Ra, Latent Membrane Protein 1 (LMP1), Neural cell-adhesion molecule (N-CAM/CD56), and Trail Receptor (TRAIL R). It is understood that these or other cancer antigens can be utilized for targeting by a cancer antigen CAR. While some exemplary suitable CARs and some exemplary suitable CAR target antigens are disclosed herein, additional suitable CARS and CAR target antigens will be apparent to the skilled artisan based on the present disclosure in view of the knowledge in the art regarding CARs and CAR antigens. See, e.g., International PCT Application PCT/US2017/027601, the entire contents of which are incorporated herein by reference.

### Cells

Some aspects of this disclosure provide cells, e.g., immune effector cells or hematopoietic stem or progenitor cells, comprising a nucleic acid constructs comprising a first expression cassette comprising a nucleic acid encoding a tetracycline-responsive transactivator under the control of a cell type-specific promoter; and a second expression cassette comprising a nucleic acid encoding a transgene, e.g., a chimeric antigen receptor, under the control of a tetracycline-responsive promoter. In some embodiments, the transgene encodes a chimeric antigen receptor targeting a human antigen associated with a disease or disorder. In some embodiments, the cell type-specific promoter is a constitutive promoter. In some embodiments, the cell type specific promoter is a CD8 promoter. In some embodiments, the cells type-specific promoter is a CD3delta promoter. In some embodiments, the cells type-specific promoter is a CD3epsilon promoter. In some embodiments, the cell type-specific promoter is a CD56 promoter. In some embodiments, the cell type-specific promoter is a CD244 promoter. In some embodiments, the cell type-specific promoter is a CD94 promoter. In some embodiments, the cell type-specific promoter is an NKG2D promoter. In some embodiments, the tetracycline-responsive transactivator is an rtTA protein. In some embodiments, the tetracycline-responsive transactivator is an rtTA protein. In some embodiments, the tetracycline-responsive transactivator is an rtTA2^{S}-M2 protein. In some embodiments, the tetracycline-responsive transactivator is an rtTA-V10 protein. In some embodiments, the transgene encodes a chimeric antigen receptor comprising a binding domain, a hinge domain, a transmembrane domain, at least one co-stimulatory domain, a cytoplasmic signaling domain, or a combination thereof. In some embodiments, the binding domain comprises an antibody, or an antigen-binding antibody fragment, that binds an antigen. In some embodiments, the binding domain comprises an scFv or a single domain antibody that binds to an antigen. In some embodiments, the antigen is a lineage-specific cell-surface antigen. In some embodiments, expression of the antigen is associated with a neoplastic or malignant disease. In some embodiments, the antigen is CD33, CD123, CD19, or CLL-1. In some embodiments, the hinge domain of the CAR is a CD8a (CD8alpha) hinge domain. In some embodiments, the transmembrane domain of the CAR is a CD8 or CD28 transmembrane domain. In some embodiments, the costimulatory domain of the CAR is a 4-1BB or CD28 costimulatory domain, or a combination thereof. In some embodiments, the cytoplasmic signaling domain of the CAR is a CD3 (CD3zeta) cytoplasmic signaling domain. In some embodiments, the cells are hematopoietic stem cells. In some embodiments, the cells are hematopoietic progenitor cell. In some embodiments, the cells are immune effector cells. In some embodiments, the cells are T-cells. In some embodiments, the cells are alpha/beta T-cells. In some embodiments, the cells are gamma/delta T-cells. In some embodiments, the cells are natural killer (NK) cells. In some embodiments, the cells are natural killer T-cells (NKT cells).

In some embodiments, the immune effector cells provided herein are T cells. In some embodiments, the T cells express the T cell receptor (TCR), e.g., the TCR α and β chains. In some embodiments, the T cells express the TCR γ and δ chains. In some embodiments, the T cells are CD4⁺ and/or CD8⁺. In some embodiments, the T cells are T helper cells (CD4⁺). In some embodiments, the T-cells are cytotoxic T cells (also referred to as cytotoxic T lymphocytes, CTL; CD8⁻ T cells). In some embodiments, the T-cells are central memory T cells (TCM), stem memory T cells (TSCM), stem-cell-like memory T cells (or stem-like memory T cells), and effector memory T cells, for example, T_{EM} cells and T_{EMRA} (CD45RA⁺) cells, effector T cells, or T helper cells, e.g., Th1 cells, Th2 cells, Th9 cells, Th17 cells, Th22 cells, Tfh (follicular helper) cells, T regulatory cells, natural killer T cells, mucosal associated invariant T cells (MAIT), and γδ T cells.

In some embodiments, T cells are isolated by methods well known in the art, including commercially available isolation methods (see, for example, Rowland-Jones et al., Lymphocytes: A Practical Approach, Oxford University Press, New York (1999)). In some embodiments, the T cells are sourced from peripheral blood, umbilical cord blood, bone marrow, or any other suitable source of hematopoietic cells.

T cell separation and isolation methods are well known in the art, e.g., T cells can be isolated using various cell surface markers or combinations of markers, depending on the desired T-cell subtype, including but not limited to, CD3, CD4, CD8, CD34 (for hematopoietic stem and progenitor cells) and the like, can be used to separate the cells, as is well known in the art (see Kearse, T Cell Protocols: Development and Activation, Humana Press, Totowa N.J. (2000); De Libero, T Cell Protocols, Vol. 514 of Methods in Molecular Biology, Humana Press, Totowa N.J. (2009); Su et al., Methods Mol. Biol. 806:287-299 (2012); Bluestone et al., Sci. Transl. Med. 7(315) (doi: 10.1126/scitranslmed.aad4134)(2015); Miyara et al., Nat. Rev. Rheumatol. 10:543-551 (2014); Liu et al., J. Exp. Med. 203:1701-1711 (2006); Seddiki et al., J. Exp. Med. 203:1693-1700 (2006); Ukena et al., Exp. Hematol. 39:1152-1160 (2011); Chen et al., J. Immunol. 183:4094-4102 (2009); Putnam et al., Diabetes 58:652-662 (2009); Putnam et al., Am. Tranplant. 13:3010-3020 (2013); Lee et al., Cancer Res. 71:2871-2881 (2011); MacDonald et al., J Clin. Invest. 126:1413-1424 (2016)). Methods for isolating and expanding regulatory T cells are also commercially available (see, for example, BD Biosciences, San Jose, Calif.; STEMCELL Technologies Inc., Vancouver, Canada; eBioscience, San Diego, Calif.; Invitrogen, Carlsbad, Calif.).

In some embodiments, the T cells are autologous to a subject to which they are administered back, e.g., after being contacted with a vector comprising an expression system provided herein. In some embodiments, the T cells are non-autologous, e.g., allogeneic to a subject to which they are administered back, e.g., after being contacted with a vector comprising an expression system provided herein.

In some embodiments, T cells are obtained from a subject, genetically engineered to comprise a cell-type specific, inducible expression system provided herein, and then administered back to the same subject. In some embodiments, T cells are obtained from a subject, genetically engineered to comprise a cell-type specific, inducible expression system provided herein, and then administered back to a different subject, e.g., an HLA-matched subject.

Any suitable method for isolating T cells that can be used for recombinant expression of a CAR can be used, including, but not limited to, methods known in the art, e.g., those described in Sadelain et al., Nat. Rev. Cancer 3:35-45 (2003); Morgan et al., Science 314:126-129 (2006), Panelli et al., J Immunol. 164:495-504 (2000); Panelli et al., J Immunol. 164:4382-4392 (2000)), Dupont et al., Cancer Res. 65:5417-5427 (2005); Papanicolaou et al., Blood 102:2498-2505 (2003)).

In some embodiments, hematopoietic stem cells (HSCs) are obtained from a subject, genetically engineered to comprise a cell-type specific, inducible expression system provided herein, and then administered back to the same subject. In some embodiments, hematopoietic stem cells (HSCs) are obtained from a subject, genetically engineered to comprise a cell-type specific, inducible expression system provided herein, and then administered back to a different subject, e.g., an HLA matched subject.

Any suitable method for isolating T cells that can be used for recombinant expression of a CAR can be used, including, but not limited to, methods known in the art, e.g., those described in Klug et al., Hematopoietic Stem Cell Protocols, Humana Press, New Jersey (2002); Freshney et al., Culture of Human Stem Cells, John Wiley & Sons (2007)).

Delivery of the gene expression systems provided herein to cells can be via any suitable method or technology. For delivery to mature immune effector cells, viral vector systems are particularly suitable, but other delivery modalities can be used as well. In some embodiments, e.g., where HSCs are genetically engineered to harbor an expression system as provided herein, a targeted integration approach can be advantageous, e.g., a targeted integration of a donor DNA comprising a cell-type specific inducible expression system provided herein, via homology-directed repair (HDR). Methods and vectors for creating targeted integration via HDR-mediated repair are known in the art, and include using RNA-directed endonucleases, such as CRISPR/Cas endonucleases, to create a DNA strand break, which in the presence of a suitable donor DNA is repaired to include the donor DNA. Other methods of creating targeted integrations are known in the art, and include, without limitation, the use of transposons or viral vectors (e.g., AAV vectors) that exhibit site specificity or site preference for integrating into the genome of a host cell. While some exemplary methods of delivery of cell-type specific inducible expression systems are provided herein, additional suitable systems will be apparent to the skilled artisan based on the present disclosure and the knowledge in the art. The disclosure is not limited in this respect.

### Methods of Use

Some aspects of this disclosure provide methods, comprising administering a plurality of cells provided herein to a subject in need thereof. For example, some aspects of this disclosure provide methods comprising administering a plurality, and preferably an effective number, of cells provided herein, e.g., immune effector cells or hematopoietic stem or progenitor cells that comprise a nucleic acid constructs comprising a first expression cassette comprising a nucleic acid encoding a tetracycline-responsive transactivator under the control of a cell type-specific promoter; and a second expression cassette comprising a nucleic acid encoding a transgene, e.g., a chimeric antigen receptor, under the control of a tetracycline-responsive promoter, to a subject in need thereof. In some embodiments, the subject has been diagnosed or has a neoplastic or malignant disease and the transgene encoded by the nucleic acid construct comprised in the cell is a CAR targeting an antigen associated with the neoplastic or malignant disease. In some embodiments, the cells are CAR-T cells. In some embodiments, the cells are CAR-NK cells. In some embodiments, the cells are HSCs or HPCs. In some embodiments, the methods further comprise administering to the subject an effective amount of tetracycline resulting in induction of the expression of the transgene. In some embodiments, the methods further comprise measuring expression of the transgene in a cell obtained from the subject. In some embodiments, the methods further comprise monitoring at least one symptom of the neoplastic or malignant disease and continuing administration of the tetracycline until the at least one symptom of the neoplastic or malignant disease is ameliorated.

Some aspects of this disclosure provide methods of administering a cell provided herein, e.g., an immune cell expressing a CAR targeting an antigen associated with a neoplastic or malignant disease or disorder, from a cell-type specific and inducible expression construct provided herein, to a subject having the neoplastic or malignant disease or disorder.

In some embodiments, administration of the cells to the subject ameliorates a sign or symptom associated with the neoplastic disease or disorder which may include, e.g., reducing the number of neoplastic or malignant cells, reducing tumor burden, including inhibiting growth of a tumor, slowing the growth rate of a tumor, reducing the size of a tumor, reducing the number of tumors, eliminating a tumor, or reducing or ameliorating a symptom associated with the neoplastic disease or disorder, e.g., fatigue, pain, weight loss, and other clinical

In some embodiments, the subject is a mammal. In some embodiments, the subject is a human subject.

In some embodiments, the subject has or has been diagnosed with a cancer. In some embodiments, the cancer is a carcinoma. In some embodiments, the cancer is a sarcoma. In some embodiments, the cancer is a leukemia. In some embodiments, the cancer is a lymphoma. In some embodiments, the cancer is multiple myeloma. In some embodiments, the cancer is a melanoma. In some embodiments, the cancer is a brain or spinal cord tumor. In some embodiments, the cancer is a germ cell tumor. In some embodiments, the cancer is a neuroendocrine tumor. In some embodiments, the cancer is a carcinoid tumor. In some embodiments, the cancer is a cancer of a hematopoietic lineage. In some embodiments, the cancer is metastatic cancer. In some embodiments, the cancer is acute myeloid leukemia (AML).

In some embodiments, the neoplastic disease is myelodysplastic syndrome (MDS).

Depending on the CAR employed in the expression systems and cell provided herein, various neoplastic diseases or malignancies can be treated, including, but not limited to bone cancer, intestinal cancer, liver cancer, skin cancer, cancer of the head or neck, melanoma (cutaneous or intraocular malignant melanoma), renal cancer (for example, clear cell carcinoma), throat cancer, prostate cancer (for example, hormone refractory prostate adenocarcinoma), blood cancers (for example, leukemias, lymphomas, and myelomas), uterine cancer, rectal cancer, cancer of the anal region, bladder cancer, brain cancer, stomach cancer, testicular cancer, carcinoma of the fallopian tubes, carcinoma of the endometrium, carcinoma of the cervix, carcinoma of the vagina, carcinoma of the vulva, leukemias (for example, acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease, Waldenstrom's macroglobulinemia), cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, heavy chain disease, and solid tumors such as sarcomas and carcinomas, for example, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilm's tumor, cervical cancer, uterine cancer, testicular cancer, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, retinoblastoma, malignant pleural disease, mesothelioma, lung cancer (for example, non-small cell lung cancer), pancreatic cancer, ovarian cancer, breast cancer (for example, metastatic breast cancer, metastatic triple-negative breast cancer), colon cancer, pleural tumor, glioblastoma, esophageal cancer, gastric cancer, and synovial sarcoma. Solid tumors can be primary tumors or tumors in a metastatic state.

In some embodiments, genetically engineered cells as provided herein, e.g., HSCs, T cells, or NK cells comprising a cell-type specific, inducible expression system driving expression of a CAR, are administered to a subject in need thereof at a dose of about 10⁴ to about 10¹⁰ cells/kg of body weight of the subject, for example, about 10⁵ to about 10⁹, about 10⁵ to about 10⁸, about 10⁵ to about 10⁷, or about 10⁵ to 10⁶. In general, in the case of systemic administration, a higher dose is used than in regional administration, where cells provided herein are administered into an organ or a tumor. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 1×10⁸, 6×10⁴, to 1×10⁸, 7×10⁴ to 1×10⁸, 8×10⁴ to 1×10⁸, 9×10⁴ to 1×10⁸, 1×10⁵ to 1×10⁸, for example, 1×10⁵ to 5×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10 5 to 7×10⁶, 1×10⁵ to 6×10⁶, 1×10⁵ to 5×10⁶, 1×10⁵ to 4×10⁶, 1×10⁵ to 3×10⁶, 1×10⁵ to 2×10⁶, 2×10⁵ to 7×10⁶, 2×10⁵ to 6×10⁶, 2×10⁵ to 5×10⁶, 2×10⁵ to 4×10⁶, 3×10⁵ to 3×10⁶, and the like. Such dose ranges can be particularly useful for regional administration. In a particular embodiment, cells are provided in a dose of 1×10⁵ to 5×10⁶, in particular 1×10⁵ to 3×10⁶ or 3×10⁵ to 3×10⁶ cells/kg for regional administration, for example, intrapleural administration. Exemplary dose ranges also can include, but are not limited to, 5×10⁵ to 1×10⁸, for example, 6×10⁵ to 1×10⁸, 7×10⁵ to 1×10⁸, 8×10⁵ to 1×10⁸, 9×10⁵ to 1×10⁸, 1×10⁶ to 1×10⁸, 1×10⁶ to 9×10⁷, 1×10⁶ to 8×10⁷, 1×10⁶ to 7×10⁷, 1×10⁶ to 6×10⁷, 1×10⁶ to 5×10⁷, 1×10⁶ to 4×10⁷, 1×10⁶ to 3×10⁷, and the like. Such does can be particularly useful for systemic administration. In a particular embodiment, cells are provided in a dose of 1×10⁶ to 3×10⁷ cells/kg for systemic administration. Exemplary cell doses include, but are not limited to, a dose of 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸ 81×10⁹ and so forth, in the range of about 10⁴ to about 10¹⁰. In addition, the dose can also be adjusted to account for whether a single dose is being administered or whether multiple doses are being administered. The precise determination of what would be considered an effective dose can be based on factors individual to each subject, including their size, age, sex, weight, and condition of the particular subject, as described above. Dosages can be readily determined by those skilled in the art based on the disclosure herein and knowledge in the art.

The genetically engineered cells provided herein can be administered by any methods known in the art, including, but not limited to, pleural administration, intravenous administration, subcutaneous administration, intranodal administration, intratumoral administration, intrathecal administration, intrapleural administration, intraperitoneal administration, intracranial administration, and direct administration to the thymus.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: T-Cell Specific Expression of a CD33 CAR

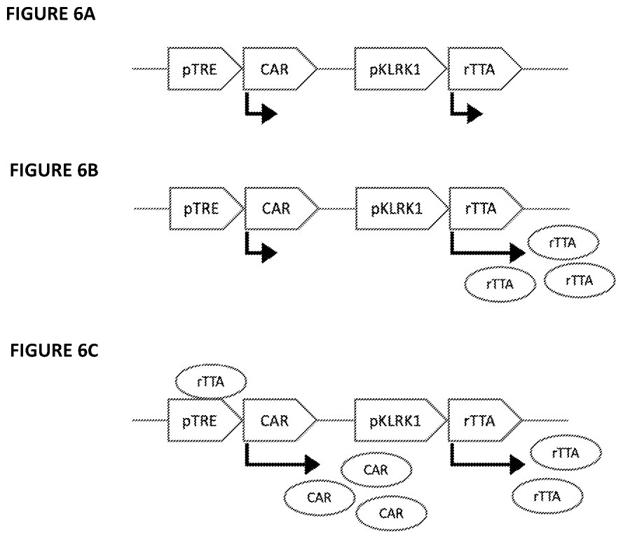

A lentiviral expression vector for inducible expression of a CD33 CAR in T-lymphocytes was designed. A schematic of the expression construct is provided in FIGS. 6A-6C.

Briefly, the lentiviral vector comprises a pLVX vector backbone (Takara Bio USA), an expression cassette comprising a nucleic acid sequence encoding a tet-responsive transactivator (rTTA 3G) under the control of a CD8 promoter (pCD8), and an expression cassette comprising a nucleic acid sequence encoding a CD33 CAR under the control of a tet-responsive promoter (pTRE). The expression cassette comprising the nucleic acid sequence encoding a CD33 CAR under the control of a tet-responsive promoter (pTRE) is oriented in a manner in which the direction of transcription of the CD33 CAR-encoding sequence is towards the 5′ LTR of the lentiviral vector, whereas the expression cassette comprising the nucleic acid sequence encoding the rTTA 3G under the control of the CD8 promoter is oriented in a manner in which the direction of transcription of the rTTA 3G-encoding sequence is towards the 3′ LTR of the lentiviral vector. The orientation of the CD33 CAR expression cassette towards the 5′LTR of the viral backbone allows for the use of a directional polyadenylation signal (pA), which does not interfere with transcription of the viral genome during packaging, but efficiently mediates polyadenylation and thus stabilization of the CD33 CAR transcript driven by the pTRE promoter. The backbone further comprises additional lentiviral elements, e.g., packaging signal (psi, or Ψ), Rev-response element (RRE), central polypurine tract/central termination sequence (is cloned into the pLVX-Tet3G vector (Clontech Laboratories/Takara Bio USA)

The exemplary sequences used in the vector design are provided below:

pTRE3G nucleic acid sequence
(SEQ ID NO: 1)
GAGTTTACTCCCTATCAGTGATAGAGAACGTATGAAGAGTTTACTCCCTATCAGTGATAGAGAACGTATGCAGAC
TTTACTCCCTATCAGTGATAGAGAACGTATAAGGAGTTTACTCCCTATCAGTGATAGAGAACGTATGACCAGTTT
ACTCCCTATCAGTGATAGAGAACGTATCTACAGTTTACTCCCTATCAGTGATAGAGAACGTATATCCAGTTTACT
CCCTATCAGTGATAGAGAACGTATAAGCTTTAGGCGTGTACGGTGGGCGCCTATAAAAGCAGAGCTCGTTTAGTG
AACCGTCAGATCGCCTGGAGCAATTCCACAACACTTTTGTCTTATACCAACTTTCCGTACCACTTCCTACCCTCG
TAAA
Kozak sequence
GCCACC
Anti-CD33 CAR-encoding nucleotide sequence
(SEQ ID NO: 2)
ATGGCTCTGCCCGTCACAGCTCTGCTGCTGCCTCTGGCCCTGCTGCTGCACGCCGCCAGACCTCAGGTGCAGCTC
GTGCAGAGCGGCGCTGAGGTGAAGAAACCTGGCAGCAGCGTGAAGGTGAGCTGCAAGGCCTCCGGCTACACCTTC
ACCGACTACAACATGCACTGGGTGAGGCAAGCCCCTGGCCAGGGACTGGAGTGGATCGGCTACATCTACCCTTAC
AACGGCGGCACAGGCTACAACCAGAAGTTCAAGTCCAAGGCCACCATCACCGCCGATGAGTCCACCAATACCGCC
TACATGGAGCTCAGCAGCCTGAGGTCCGAGGACACAGCCGTCTACTACTGCGCCAGGGGCAGGCCCGCTATGGAC
TACTGGGGCCAGGGCACCCTGGTGACAGTGAGCTCTGGTGGCGGCGGATCCGGCGGCGGCGGCAGCGGCGGCGGC
GGCTCCGACATTCAGATGACCCAGAGCCCTAGCAGCCTGAGCGCTTCCGTGGGAGACAGGGTGACCATCACATGC
AGGGCCTCCGAGAGCGTGGACAATTACGGCATCAGCTTCATGAACTGGTTCCAGCAGAAGCCCGGCAAGGCCCCC
AAACTGCTGATCTATGCCGCCAGCAATCAGGGCTCCGGCGTGCCTAGCAGGTTTTCCGGCAGCGGCAGCGGCACC
GACTTTACCCTGACCATCTCCAGCCTGCAGCCTGACGATTTCGCCACCTACTACTGCCAGCAGAGCAAGGAGGTG
CCTTGGACCTTTGGACAGGGCACAAAGGTGGAGATCAAGTCCGGAACCACGACGCCAGCGCCGCGACCACCAACA
CCGGCGCCCACCATCGCGTCGCAGCCCCTGTCCCTGCGCCCAGAGGCGTGCCGGCCAGCGGCGGGGGGCGCAGTG
CACACGAGGGGGCTGGACTTCGCCTGTGATATCTACATCTGGGCGCCCTTGGCCGGGACTTGTGGGGTCCTTCTC
CTGTCACTGGTTATCACCCTTTACTGCAAACGGGGCAGAAAGAAACTCCTGTATATATTCAAACAACCATTTATG
AGACCAGTACAAACTACTCAAGAGGAAGATGGCTGTAGCTGCCGATTTCCAGAAGAAGAAGAAGGAGGATGTGAA
CTGAGAGTGAAGTTCAGCAGGAGCGCAGACGCCCCCGCGTACAAGCAGGGCCAGAACCAGCTCTATAACGAGCTC
AATCTAGGACGAAGAGAGGAGTACGATGTTTTGGACAAGAGACGTGGCCGGGACCCTGAGATGGGGGGAAAGCCG
AGAAGGAAGAACCCTCAGGAAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTCTCAGTACAGCCACCAAGGAC
ACCTACGACGCCCTTCACATGCAGGCCCTGCCCCCTCGCTGA
Anti-CD33 CAR amino acid sequence
(SEQ ID NO: 3)
MALPVTALLLPLALLLHAARPQVQLVQSGAEVKKPGSSVKVSCKASGYTFTDYNMHWVRQAPGQGLEWIGYIYPY
NGGTGYNQKFKSKATITADESTNTAYMELSSLRSEDTAVYYCARGRPAMDYWGQGTLVTVSSGGGGSGGGGSGGG
GSDIQMTQSPSSLSASVGDRVTITCRASESVDNYGISFMNWFQQKPGKAPKLLIYAASNQGSGVPSRFSGSGSGT
DFTLTISSLQPDDFATYYCQQSKEVPWTFGQGTKVEIKSGTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEEDGCSCRFPEEEEGGCE
LRVKFSRSADAPAYKOGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
SV40 polyA signal
(SEQ ID NO: 4)
AACTTGTTTATTGCAGCTTATAATGGTTACAAATAAAGCAATAGCATCACAAATTTCACAAATAAAGCATTTTTT
TCACTGCATTCTAGTTGTGGTTTGTCCAAACTCATCAATGTATCTTA
CD8a promoter sequence
(SEQ ID NO: 5)
TTCTACCCACCATTAAAGCTTTGGCATTCTGGCCATTTCTTTCTTTTTAAAAAATTATTATTATTATTTTTTATT
TTGAGATGGGAGTCTCGCTCTATCCCCCAGCCTGGAGTGCAGTGACTCGATCTCCGCTCGCTGCAACTTCCGCCT
CCAGGGTTCAAACGATTCTTCTGCCTCAGCCTCCCCAGTAGCTAGGACTACAGGCTTGCGCCATCACGCCCGGCT
AGTTTTTTTTTTGTTTTTTGTTTGTTTGTTTGTTTGTTTTTAAAGTAGAAACAGGATTTCGCCATATTGGCCAGG
CTGGTCTCGAACTCCTGACCTCAGTTGATCCGACCGCCTCGGCCTCCCAAAGTGTTGGAATTACAGCAGTGAGCC
ACCGCGCACCAAGCCCGGCCCATTCTGGCCATATCTATAGACGGATGCATTGCACAGGAGGCTCAGCACTAATCG
GTAGATACTGCGAGATGCTGGGAGGTTAAGGGGCCTACCCGCAATATCTCTGGCCAATGCCTTGGGCTAGAAATG
CCATAATTAGCCGCTCTTTTGATCCCTTGCAAAATGCGAATCCCACCGCACCTCCACCCCACCCGAGTGGTAATC
TCCTAGTGGTAATCTAAGTGAGCCTGTGATAAGATAAGTAGCTCCTGGTGGTGAGGGTGAGAAATTGGGGAGCTG
GAGCCCCAGCCAGGGACGAGGCTGTAGGGGCTAGGGCGAAGATGGAGGCTGCTGGGCCCCCAGATGGAAGACGGT
AACGTGCGCCCGCTTCGTTTTTGCTCGAGGTCAGTCAGGTGCAGACTGAATTCGAAGTCGCTCCCTCCTCCGCTC
AACCCCGACCAGGCCAAAACTAAAGCAGCACCGCCCCCTGCTGGGCCGACAGGGCATCAGATTTTGCTGGACGCG
GGTGACAGGCGAGATAGGGAGTGTCCCTGCTGCTAGTGCCCCTGCTGCTAGTGCCTAGTTACCTGCAGTACTTAC
CCTCCCTGGGCCTCAGTTTTTCTTC
rTTA 3G nucleic acid sequence
(SEQ ID NO: 6)
CCCCTCTCCCTCCCCCCCCCCTAACGTTACTGGCCGAAGCCGCTTGGAATAAGGCCGGTGTGCGTTTGTCTATAT
GTTATTTTCCACCATATTGCCGTCTTTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATGTCGTGAAGGAAGCAGTTCCTCTGGA
AGCTTCTTGAAGACAAACAACGTCTGTAGCGACCCTTTGCAGGCAGCGGAACCCCCCACCTGGCGACAGGTGCCT
CTGCGGCCAAAAGCCACGTGTATAAGATACACCTGCAAAGGCGGCACAACCCCAGTGCCACGTTGTGAGTTGGAT
AGTTGTGGAAAGAGTCAAATGGCTCTCCTCAAGCGTATTCAACAAGGGGCTGAAGGATGCCCAGAAGGTACCCCA
TTGTATGGGATCTGATCTGGGGCCTCGGTGCACATGCTTTACATGTGTTTAGTCGAGGTTAAAAAAACGTCTAGG
CCCCCCGAACCACGGGGACGTGGTTTTCCTTTGAAAAACACGATGATAAGCTTGCCACAACCATGGCTGAACAAG
ATGGATTGCACGCAGGTTCTCCGGCCGCTTGGGTGGAGAGGCTATTCGGCTATGACTGGGCACAACAGACAATCG
GCTGCTCTGATGCCGCCGTGTTCCGGCTGTCAGCGCAGGGGCGCCCGGTTCTTTTTGTCAAGACCGACCTGTCCG
GTGCCCTGAATGAACTGCAGGACGAGGCAGCGCGGCTATCGTGGCTGGCCACGACGGGCGTTCCTTGCGCAGCTG
TGCTCGACGTTGTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCTTGATCCGGCTA
CCTGCCCATTCGACCACCAAGCGAAACATCGCATCGAGCGAGCACGTACTCGGATGGAAGCCGGTCTTGTCGATC
AGGATGATCTGGACGAAGAGCATCAGGGGCTCGCGCCAGCCGAACTGTTCGCCAGGCTCAAGGCGCGCATGCCCG
ACGGCGAGGATCTCGTCGTGACCCATGGCGATGCCTGCTTGCCGAATATCATGGTGGAAAATGGCCGCTTTTCTG
GATTCATCGACTGTGGCCGGCTGGGTGTGGCGGACCGCTATCAGGACATAGCGTTGGCTACCCGTGATATTGCTG
AAGAGCTTGGCGGCGAATGGGCTGACCGCTTCCTCGTGCTTTACGGTATCGCCGCTCCCGATTCGCAGCGCATCG
CCTTCTATCGCCTTCTTGACGAGTTCTTCTGA
rTTA 3G amino acid sequence
(SEQ ID NO: 7)
MSRLDKSKVINSALELLNGVGIEGLTTRKLAQKLGVEQPTLYWHVKNKRALLDALPIEMLDRHHTHSCPLEGESW
QDFLRNNAKSYRCALLSHRDGAKVHLGTRPTEKQYETLENQLAFLCQQGFSLENALYALSAVGHFTLGCVLEEQE
HQVAKEERETPTTDSMPPLLKQAIELFDRQGAEPAFLFGLELIICGLEKQLKCESGGPTDALDDFDLDMLPADAL
DDFDLDMLPADALDDFDLDMLPG
Lentiviral Vector Sequence
(SEQ ID NO: 8)
TGGAAGGGCTAATTCACTCCCAAAGAAGACAAGATATCCTTGATCTGTGGATCTACCACACACAAGGCTACTTCC
CTGATTAGCAGAACTACACACCAGGGCCAGGGGTCAGATATCCACTGACCTTTGGATGGTGCTACAAGCTAGTAC
CAGTTGAGCCAGATAAGGTAGAAGAGGCCAATAAAGGAGAGAACACCAGCTTGTTACACCCTGTGAGCCTGCATG
GGATGGATGACCCGGAGAGAGAAGTGTTAGAGTGGAGGTTTGACAGCCGCCTAGCATTTCATCACGTGGCCCGAG
AGCTGCATCCGGAGTACTTCAAGAACTGCTGATATCGAGCTTGCTACAAGGGACTTTCCGCTGGGGACTTTCCAG
GGAGGCGTGGCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGCTTAAGCCT
CAATAAAGCTTGCCTTGAGTGCTTCAAGTAGTGTGTGCCCGTCTGTTGTGTGACTCTGGTAACTAGAGATCCCTC
AGACCCTTTTAGTCAGTGTGGAAAATCTCTAGCAGTGGCGCCCGAACAGGGACTTGAAAGCGAAAGGGAAACCAG
AGGAGCTCTCTCGACGCAGGACTCGGCTTGCTGAAGCGCGCACGGCAAGAGGCGAGGGGCGGCGACTGGTGAGTA
CGCCAAAAATTTTGACTAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTAAAACATATAGTATGGG
CAAGCAGGGAGCTAGAACGATTCGCAGTTAATCCTGGCCTGTTAGAAACATCAGAAGGCTGTAGACAAATACTGG
GACAGCTACAACCATCCCTTCAGACAGGATCAGAAGAACTTAGATCATTATATAATACAGTAGCAACCCTCTATT
GTGTGCATCAAAGGATAGAGATAAAAGACACCAAGGAAGCTTTAGACAAGATAGAGGAAGAGCAAAACAAAAGTA
AGACCACCGCACAGCAAGCGGCCGGCCGCTGATCTTCAGACCTGGAGGAGGAGATATGAGGGACAATTGGAGAAG
TGAATTATATAAATATAAAGTAGTAAAAATTGAACCATTAGGAGTAGCACCCACCAAGGCAAAGAGAAGAGTGGT
GCAGAGAGAAAAAAGAGCAGTGGGAATAGGAGCTTTGTTCCTTGGGTTCTTGGGAGCAGCAGGAAGCACTATGGG
CGCAGCGTCAATGACGCTGACGGTACAGGCCAGACAATTATTGTCTGGTATAGTGCAGCAGCAGAACAATTTGCT
GAGGGCTATTGAGGCGCAACAGCATCTGTTGCAACTCACAGTCTGGGGCATCAAGCAGCTCCAGGCAAGAATCCT
GGCTGTGGAAAGATACCTAAAGGATCAACAGCTCCTGGGGATTTGGGGTTGCTCTGGAAAACTCATTTGCACCAC
TGCTGTGCCTTGGAATGCTAGTTGGAGTAATAAATCTCTGGAACAGATTTGGAATCACACGACCTGGATGGAGTG
GGACAGAGAAATTAACAATTACACAAGCTTAATACACTCCTTAATTGAAGAATCGCAAAACCAGCAAGAAAAGAA
TGAACAAGAATTATTGGAATTAGATAAATGGGCAAGTTTGTGGAATTGGTTTAACATAACAAATTGGCTGTGGTA
TATAAAATTATTCATAATGATAGTAGGAGGCTTGGTAGGTTTAAGAATAGTTTTTGCTGTACTTTCTATAGTGAA
TAGAGTTAGGCAGGGATATTCACCATTATCGTTTCAGACCCACCTCCCAACCCCGAGGGGACCCGACAGGCCCGA
AGGAATAGAAGAAGAAGGTGGAGAGAGAGACAGAGACAGATCCATTCGATTAGTGAACGGATCTCGACGGTATCG
CCTTTAAAAGAAAAGGGGGGATTGGGGGGTACAGTGCAGGGGAAAGAATAGTAGACATAATAGCAACAGACATAC
AAACTAAAGAATTACAAAAACAAATTACAAAAATTCAAAATTTTCGGGTTTATTACAGGGACAGCTAAGATACAT
TGATGAGTTTGGACAAACCACAACTAGAATGCAGTGAAAAAAATGCTTTATTTGTGAAATTTGTGATGCTATTGC
TTTATTTGTAACCATTATAAGCTGCAATAAACAAGTTAGAGATCCAGTTTATTTCAGCGAGGGGGCAGGGCCTGC
ATGTGAAGGGCGTCGTAGGTGTCCTTGGTGGCTGTACTGAGACCCTGGTAAAGGCCATCGTGCCCCTTGCCCCTC
CGGCGCTCGCCTTTCATCCCAATCTCACTGTAGGCCTCCGCCATCTTATCTTTCTGCAGTTCATTGTACAGGCCT
TCCTGAGGGTTCTTCCTTCTCGGCTTTCCCCCCATCTCAGGGTCCCGGCCACGTCTCTTGTCCAAAACATCGTAC
TCCTCTCTTCGTCCTAGATTGAGCTCGTTATAGAGCTGGTTCTGGCCCTGCTTGTACGCGGGGGCGTCTGCGCTC
CTGCTGAACTTCACTCTCAGTTCACATCCTCCTTCTTCTTCTTCTGGAAATCGGCAGCTACAGCCATCTTCCTCT
TGAGTAGTTTGTACTGGTCTCATAAATGGTTGTTTGAATATATACAGGAGTTTCTTTCTGCCCCGTTTGCAGTAA
AGGGTGATAACCAGTGACAGGAGAAGGACCCCACAAGTCCCGGCCAAGGGCGCCCAGATGTAGATATCACAGGCG
AAGTCCAGCCCCCTCGTGTGCACTGCGCCCCCCGCCGCTGGCCGGCACGCCTCTGGGCGCAGGGACAGGGGCTGC
GACGCGATGGTGGGCGCCGGTGTTGGTGGTCGCGGCGCTGGCGTCGTGGTTCCGGACTTGATCTCCACCTTTGTG
CCCTGTCCAAAGGTCCAAGGCACCTCCTTGCTCTGCTGGCAGTAGTAGGTGGCGAAATCGTCAGGCTGCAGGCTG
GAGATGGTCAGGGTAAAGTCGGTGCCGCTGCCGCTGCCGGAAAACCTGCTAGGCACGCCGGAGCCCTGATTGCTG
GCGGCATAGATCAGCAGTTTGGGGGCCTTGCCGGGCTTCTGCTGGAACCAGTTCATGAAGCTGATGCCGTAATTG
TCCACGCTCTCGGAGGCCCTGCATGTGATGGTCACCCTGTCTCCCACGGAAGCGCTCAGGCTGCTAGGGCTCTGG
GTCATCTGAATGTCGGAGCCGCCGCCGCCGCTGCCGCCGCCGCCGGATCCGCCGCCACCAGAGCTCACTGTCACC
AGGGTGCCCTGGCCCCAGTAGTCCATAGCGGGCCTGCCCCTGGCGCAGTAGTAGACGGCTGTGTCCTCGGACCTC
AGGCTGCTGAGCTCCATGTAGGCGGTATTGGTGGACTCATCGGCGGTGATGGTGGCCTTGGACTTGAACTTCTGG
TTGTAGCCTGTGCCGCCGTTGTAAGGGTAGATGTAGCCGATCCACTCCAGTCCCTGGCCAGGGGCTTGCCTCACC
CAGTGCATGTTGTAGTCGGTGAAGGTGTAGCCGGAGGCCTTGCAGCTCACCTTCACGCTGCTGCCAGGTTTCTTC
ACCTCAGCGCCGCTCTGCACGAGCTGCACCTGAGGTCTGGCGGCGTGCAGCAGCAGGGCCAGAGGCAGCAGCAGA
GCTGTGACGGGCAGAGCCATGGTGGCTTACGAGGGTAGGAAGTGGTACGGAAAGTTGGTATAAGACAAAAGTGTT
GTGGAATTGCTCCAGGCGATCTGACGGTTCACTAAACGAGCTCTGCTTTTATAGGCGCCCACCGTACACGCCTAA
AGCTTATACGTTCTCTATCACTGATAGGGAGTAAACTGGATATACGTTCTCTATCACTGATAGGGAGTAAACTGT
AGATACGTTCTCTATCACTGATAGGGAGTAAACTGGTCATACGTTCTCTATCACTGATAGGGAGTAAACTCCTTA
TACGTTCTCTATCACTGATAGGGAGTAAAGTCTGCATACGTTCTCTATCACTGATAGGGAGTAAACTCTTCATAC
GTTCTCTATCACTGATAGGGAGTAAACTCCGATGGATCTATTTCCGGTGAATTCCGTAGATACTGCGAGATGCTG
GGAGGTTAAGGGGCCTACCCGCAATATCTCTGGCCAATGCCTTGGGCTAGAAATGCCATAATTAGCCGCTCTTTT
GATCCCTTGCAAAATGCGAATCCCACCGCACCTCCACCCCACCCGAGTGGTAATCTCCTAGTGGTAATCTAAGTG
AGCCTGTGATAAGATAAGTAGCTCCTGGTGGTGAGGGTGAGAAATTGGGGAGCTGGAGCCCCAGCCAGGGACGAG
GCTGTAGGGGCTAGGGCGAAGATGGAGGCTGCTGGGCCCCCAGATGGAAGACGGTAACGTGCGCCCGCTTCGTTT
TTGCTCGAGGTCAGTCAGGTGCAGACTGAATTCGAAGTCGCTCCCTCCTCCGCTCAACCCCGACCAGGCCAAAAC
TAAAGCAGCACCGCCCCCTGCTGGGCCGACAGGGCATCAGATTTTGCTGGACGCGGGTGACAGGCGAGATAGGGA
GTGTCCCTGCTGCTAGTGCCCCTGCTGCTAGTGCCTAGTTACCTGCAGTACTTACCCTCCCTGGGCCTCAGTTTT
TCTTCTCGAGGCCACCATGTCTAGACTGGACAAGAGCAAAGTCATAAACTCTGCTCTGGAATTACTCAATGGAGT
CGGTATCGAAGGCCTGACGACAAGGAAACTCGCTCAAAAGCTGGGAGTTGAGCAGCCTACCCTGTACTGGCACGT
GAAGAACAAGCGGGCCCTGCTCGATGCCCTGCCAATCGAGATGCTGGACAGGCATCATACCCACTCCTGCCCCCT
GGAAGGCGAGTCATGGCAAGACTTTCTGCGGAACAACGCCAAGTCATACCGCTGTGCTCTCCTCTCACATCGCGA
CGGGGCTAAAGTGCATCTCGGCACCCGCCCAACAGAGAAACAGTACGAAACCCTGGAAAATCAGCTCGCGTTCCT
GTGTCAGCAAGGCTTCTCCCTGGAGAACGCACTGTACGCTCTGTCCGCCGTGGGCCACTTTACACTGGGCTGCGT
ATTGGAGGAACAGGAGCATCAAGTAGCAAAAGAGGAAAGAGAGACACCTACCACCGATTCTATGCCCCCACTTCT
GAAACAAGCAATTGAGCTGTTCGACCGGCAGGGAGCCGAACCTGCCTTCCTTTTCGGCCTGGAACTAATCATATG
TGGCCTGGAGAAACAGCTAAAGTGCGAAAGCGGCGGGCCGACCGACGCCCTTGACGATTTTGACTTAGACATGCT
CCCAGCCGATGCCCTTGACGACTTTGACCTTGATATGCTGCCTGCTGACGCTCTTGACGATTTTGACCTTGACAT
GCTCCCCGGGTAAGGATCCCGACGCGTCTGGAACAATCAACCTCTGGATTACAAAATTTGTGAAAGATTGACTGG
TATTCTTAACTATGTTGCTCCTTTTACGCTATGTGGATACGCTGCTTTAATGCCTTTGTATCATGCTATTGCTTC
CCGTATGGCTTTCATTTTCTCCTCCTTGTATAAATCCTGGTTGCTGTCTCTTTATGAGGAGTTGTGGCCCGTTGT
CAGGCAACGTGGCGTGGTGTGCACTGTGTTTGCTGACGCAACCCCCACTGGTTGGGGCATTGCCACCACCTGTCA
GCTCCTTTCCGGGACTTTCGCTTTCCCCCTCCCTATTGCCACGGCGGAACTCATCGCCGCCTGCCTTGCCCGCTG
CTGGACAGGGGCTCGGCTGTTGGGCACTGACAATTCCGTGGTGTTGTCGGGGAAGCTGACGTCCTTTCCATGGCT
GCTCGCCTGTGTTGCCACCTGGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTCGCCCTCAGACGAGTCGGAT
CTCCCTTTGGGCCGCCTCCCCGCCTGGAATTAATTCTGCAGTCGAGACCTAGAAAAACATGGAGCAATCACAAGT
AGCAATACAGCAGCTACCAATGCTGATTGTGCCTGGCTAGAAGCACAAGAGGAGGAGGAGGTGGGTTTTCCAGTC
ACACCTCAGGTACCTTTAAGACCAATGACTTACAAGGCAGCTGTAGATCTTAGCCACTTTTTAAAAGAAAAGAGG
GGACTGGAAGGGCTAATTCACTCCCAACGAAGACAAGATATCCTTGATCTGTGGATCTACCACACACAAGGCTAC
TTCCCTGATTAGCAGAACTACACACCAGGGCCAGGGGTCAGATATCCACTGACCTTTGGATGGTGCTACAAGCTA
GTACCAGTTGAGCCAGATAAGGTAGAAGAGGCCAATAAAGGAGAGAACACCAGCTTGTTACACCCTGTGAGCCTG
CATGGGATGGATGACCCGGAGAGAGAAGTGTTAGAGTGGAGGTTTGACAGCCGCCTAGCATTTCATCACGTGGCC
CGAGAGCTGCATCCGGAGTACTTCAAGAACTGCTGATATCGAGCTTGCTACAAGGGACTTTCCGCTGGGGACTTT
CCAGGGAGGCGTGGCCTGGGCGGGACTGGGGAGTGGCGAGCCCTCAGATCCTGCATATAAGCAGCTGCTTTTTGC
CTGTACTGGGTCTCTCTGGTTAGACCAGATCTGAGCCTGGGAGCTCTCTGGCTAACTAGGGAACCCACTGCTTAA
GCCTCAATAAAGCTTGCCTTGAGTGCTTCAAGTAGTGTGTGCCCGTCTGTTGTGTGACTCTGGTAACTAGAGATC
CCTCAGACCCTTTTAGTCAGTGTGGAAAATCTCTAGCAGT

Annotation of certain features within the vector sequence above:

| Feature | Nucleotide position numbers: |
|---|---|
| 5′ LTR | 1 . . . 635 |
| -psi (Packaging signal) | 685 . . . 822 |
| RRE | 1303 . . . 1536 |
| cPPT/CTS | 2028 . . . 2151 |
| SV40 poly(A) signal | 2166 . . . 2287 (reverse orientation) |
| STOP codon | 2304 . . . 2306 (reverse orientation) |
| Anti CD33 CAR | 2307 . . . 3770 (reverse orientation) |
| Kozak sequence | 3771 . . . 3776 (reverse orientation) |
| TRE3G promoter | 3777 . . . 4154 (reverse orientation) |
| pCD8 | 4181 . . . 4730 |
| Tet-On 3G rTTA | 4742 . . . 5488 |
| WPRE | 5510 . . . 6101 |
| 3′ LTR | 6304 . . . 6940 |

The lentiviral vector is cloned into a cloning plasmid, e.g., pUC19, for amplification in bacteria. Amplification, purification of the lentiviral fragment, lentiviral packaging and concentration, and transduction of target cells is performed according to standard methods.

Normal CD4+/CD8+ T cells are obtained and cultured using standard protocols. T cells are transduced with lentiviral supernatant 24 hours following stimulation at a MOI of 1-5. T cells are grown in T-cell media for up to 15 days, and then cryopreserved.

HSCs are obtained from bone marrow samples selected for CD34 and cryopreserved. HSCs are transduced with lentiviral supernatant 24 hours after thawing according to standard protocols.

The MOLM14 cell line, expressing CD33, is cultured in R10 media (RPMI media, 10% fetal calf serum, penicillin, and streptomycin).

Surface expression of anti-CD33 CAR is detected by using a labeled ligand (CD33 extracellular domain) or antibody specifically binding the anti-CD33 CAR.

Cytotoxicity assays and cytokine excretion assays are performed using standard protocols. CD33 expressing cells, e.g., MOLM cells expressing luciferase, are incubated at different ratios with effector T-cells. Killing is monitored and calculated according to standard protocols. Cytokine assays are performed according to standard protocols.

HSC cells are differentiated into pre-T-cells and mature T-cells according to standard protocols. Expression of CD8 and anti-CD33 CAR is monitored during differentiation.

Mature T cells as well as pre-T cells transduced with the lentiviral vector demonstrate inducible expression of the anti-CD33 CAR, which can be induced by administration of doxycycline to the cells, and which is abolished upon withdrawal of doxycycline from the cells.

HSCs transduced with the lentiviral vector do not exhibit inducible expression of the anti-CD33 CAR, even in the presence of doxycycline. However, pre-T cells and mature T-cells differentiated from these HSCs show inducible expression of the anti-CD33 CAR upon administration of doxycycline, which is abolished upon withdrawal of doxycycline from the cells.

When administered to a suitable mouse model, the transduced HSCs engraft and populate the hematopoietic niche. After a time sufficient for mature T-lymphocytes derived from these HSCs to emerge, anti-CD33 CAR expression can be induced in these mice by injection of doxycycline.

### Example 2: T-Cell Specific Expression of a CD33 CAR

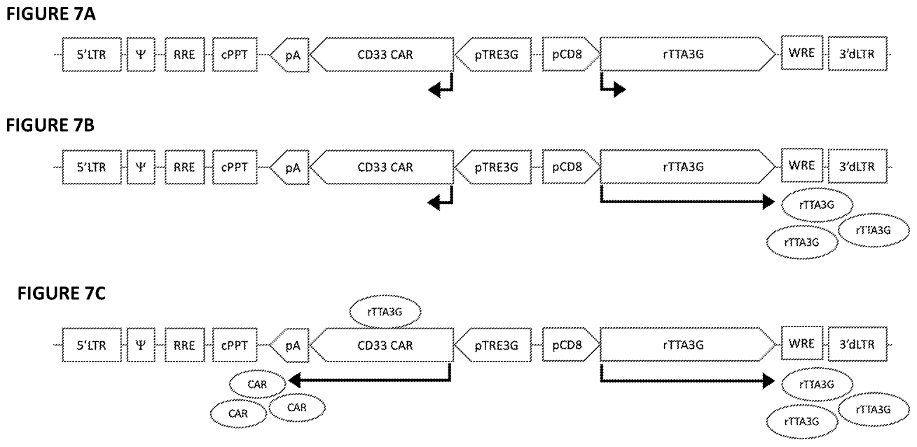

A lentiviral expression vector for inducible expression of a CD33 CAR in T-lymphocytes was designed. A schematic of the expression construct is provided in FIGS. 7A-7C.

Briefly, the lentiviral vector comprises a pLVX vector backbone (Takara Bio USA), an expression cassette comprising a nucleic acid sequence encoding a tet-responsive transactivator (rTTA 3G) under the control of a TCRbeta promoter (pTCRb), and an expression cassette comprising a nucleic acid sequence encoding a CD33 CAR under the control of a tet-responsive promoter (pTRE). The expression cassette comprising the nucleic acid sequence encoding a CD33 CAR under the control of a tet-responsive promoter (pTRE) is oriented in a manner in which the direction of transcription of the CD33 CAR-encoding sequence is towards the 5′ LTR of the lentiviral vector, whereas the expression cassette comprising the nucleic acid sequence encoding the rTTA 3G under the control of the TCRb promoter is oriented in a manner in which the direction of transcription of the rTTA 3G-encoding sequence is towards the 3′ LTR of the lentiviral vector. The orientation of the CD33 CAR expression cassette towards the 5′LTR of the viral backbone allows for the use of a directional polyadenylation signal (pA), which does not interfere with transcription of the viral genome during packaging, but efficiently mediates polyadenylation and thus stabilization of the CD33 CAR transcript driven by the pTRE promoter. The backbone further comprises additional lentiviral elements, e.g., packaging signal (psi, or Ψ), Rev-response element (RRE), central polypurine tract/central termination sequence (is cloned into the pLVX-Tet3G vector (Clontech Laboratories/Takara Bio USA).

The sequences used in the vector design are provided below:

pTRE3G nucleic acid sequence
(SEQ ID NO: 1)
GAGTTTACTCCCTATCAGTGATAGAGAACGTATGAAGAGTTTACTCCCTATCAGTGATAGAGAACGTATGCAGAC
TTTACTCCCTATCAGTGATAGAGAACGTATAAGGAGTTTACTCCCTATCAGTGATAGAGAACGTATGACCAGTTT
ACTCCCTATCAGTGATAGAGAACGTATCTACAGTTTACTCCCTATCAGTGATAGAGAACGTATATCCAGTTTACT
CCCTATCAGTGATAGAGAACGTATAAGCTTTAGGCGTGTACGGTGGGCGCCTATAAAAGCAGAGCTCGTTTAGTG
AACCGTCAGATCGCCTGGAGCAATTCCACAACACTTTTGTCTTATACCAACTTTCCGTACCACTTCCTACCCTCG
TAAA
Kozak sequence
GCCACC
Anti-CD33 CAR-encoding nucleotide sequence
(SEQ ID NO: 2)
ATGGCTCTGCCCGTCACAGCTCTGCTGCTGCCTCTGGCCCTGCTGCTGCACGCCGCCAGACCTCAGGTGCAGCTC
GTGCAGAGCGGCGCTGAGGTGAAGAAACCTGGCAGCAGCGTGAAGGTGAGCTGCAAGGCCTCCGGCTACACCTTC
ACCGACTACAACATGCACTGGGTGAGGCAAGCCCCTGGCCAGGGACTGGAGTGGATCGGCTACATCTACCCTTAC
AACGGCGGCACAGGCTACAACCAGAAGTTCAAGTCCAAGGCCACCATCACCGCCGATGAGTCCACCAATACCGCC
TACATGGAGCTCAGCAGCCTGAGGTCCGAGGACACAGCCGTCTACTACTGCGCCAGGGGCAGGCCCGCTATGGAC
TACTGGGGCCAGGGCACCCTGGTGACAGTGAGCTCTGGTGGCGGCGGATCCGGCGGCGGCGGCAGCGGCGGCGGC
GGCTCCGACATTCAGATGACCCAGAGCCCTAGCAGCCTGAGCGCTTCCGTGGGAGACAGGGTGACCATCACATGC
AGGGCCTCCGAGAGCGTGGACAATTACGGCATCAGCTTCATGAACTGGTTCCAGCAGAAGCCCGGCAAGGCCCCC
AAACTGCTGATCTATGCCGCCAGCAATCAGGGCTCCGGCGTGCCTAGCAGGTTTTCCGGCAGCGGCAGCGGCACC
GACTTTACCCTGACCATCTCCAGCCTGCAGCCTGACGATTTCGCCACCTACTACTGCCAGCAGAGCAAGGAGGTG
CCTTGGACCTTTGGACAGGGCACAAAGGTGGAGATCAAGTCCGGAACCACGACGCCAGCGCCGCGACCACCAACA
CCGGCGCCCACCATCGCGTCGCAGCCCCTGTCCCTGCGCCCAGAGGCGTGCCGGCCAGCGGCGGGGGGCGCAGTG
CACACGAGGGGGCTGGACTTCGCCTGTGATATCTACATCTGGGCGCCCTTGGCCGGGACTTGTGGGGTCCTTCTC
CTGTCACTGGTTATCACCCTTTACTGCAAACGGGGCAGAAAGAAACTCCTGTATATATTCAAACAACCATTTATG
AGACCAGTACAAACTACTCAAGAGGAAGATGGCTGTAGCTGCCGATTTCCAGAAGAAGAAGAAGGAGGATGTGAA
CTGAGAGTGAAGTTCAGCAGGAGCGCAGACGCCCCCGCGTACAAGCAGGGCCAGAACCAGCTCTATAACGAGCTC
AATCTAGGACGAAGAGAGGAGTACGATGTTTTGGACAAGAGACGTGGCCGGGACCCTGAGATGGGGGGAAAGCCG
AGAAGGAAGAACCCTCAGGAAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTCTCAGTACAGCCACCAAGGAC
ACCTACGACGCCCTTCACATGCAGGCCCTGCCCCCTCGCTGA
Anti-CD33 CAR amino acid sequence
(SEQ ID NO: 3)
MALPVTALLLPLALLLHAARPQVQLVQSGAEVKKPGSSVKVSCKASGYTFTDYNMHWVRQAPGQGLEWIGYIYPY
NGGTGYNQKFKSKATITADESTNTAYMELSSLRSEDTAVYYCARGRPAMDYWGQGTLVTVSSGGGGSGGGGSGGG
GSDIQMTQSPSSLSASVGDRVTITCRASESVDNYGISFMNWFQQKPGKAPKLLIYAASNQGSGVPSRFSGSGSGT
DFTLTISSLQPDDFATYYCQQSKEVPWTFGQGTKVEIKSGTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEEDGCSCRFPEEEEGGCE
LRVKFSRSADAPAYKQGQNQLYNELNLGRREEYDVLDKRRGRDPFMGGKPRRKNPQEGLYNELQKDKMAEAYSEI
GMKGERRRGKGHDGLYQGLSTATKDTYDALHMQALPPR
SV40 polyA signal
(SEQ ID NO: 4)
AACTTGTTTATTGCAGCTTATAATGGTTACAAATAAAGCAATAGCATCACAAATTTCACAAATAAAGCATTTTTT
TCACTGCATTCTAGTTGTGGTTTGTCCAAACTCATCAATGTATCTTA
TCRbeta promoter sequence
(SEQ ID NO: 9)
GCTTTGGTTAATGGGAAGTGGCTAATGGATAGAGGCTGAAAGAATTGGGAGGAGCAGACTAGCAAAAGTCTAGAT
TCCTGAAAACAGAGCATAAAGGGCAATTCTGGTGAAGGCTCAGGGGGAAATGAGGAACAAGGTATTGAAAATCGA
AGTAAATGCCATCCTTGTTGTAAGTAGCAAAAACCTTGGCAAAATTGTGTCCTGTTCTAGAACTTTATGGAATAT
AAAAATTATGAGCCATTTGCTAGTATATCTGCTGAAAGAAATATCTAAGAGGCAAAGCATTCAGGCTACTGTGTG
ACTACTTTTAGCCACCAGGAAATTTAACCCAGCAAGAAGGGAGCCATGGGAATAGATTTTGCAAACCAGCACAGA
TGGTGACTCTACCTCCCTCTGCTGTCCTGTCTCCCATAAGCCAAACTCTGTGCTGTGAGCTGTTAAAGTCCAAGA
ATTATTTCTGGGGAATCTGTACTCTTAATAATTTACAGACAACACAGGTCTGCTTTGGATCTGATCAGACAGACT
AAATCTTGGGGACTGTACCAGTGGCCACTGAGAAAAGGGGCTCGGAATGTTGTCTTGGATAGAAAAATATAATAA
GAAACATTGGTGTGAATCTAGCATCAGAAAGATGACGTGAGGACAGCAAGGAAGAGCTGGAAACTGAGGTTTACT
CACAGGCTCCTCACCCTCCGCTGATGGGCAGGTGTGTGAGCTCCAGCATGGAGCACCACAGCACTAGGTGGGAGA
ACGGTGATAGGGTGATGGGGCAGCCTGTGAGCTGGGGCAGTGTAGGCAGAGGAGGAACTGTATCACCACAGAAAC
TTCTGCCTTCACACATCCCTCCAGCTAGGCAGGACAGGTAGAGAGTCCAGTGTCCTGGAGCACTAGACCTAAGGA
AGGCTGCATGGGGAGGACAAAGGACAGTGACATCACAGGATACCCCTCCCATCAGGAAAATCAAGGCCCAGAACT
CACTCGGCTCTTCCCCAGGAGAACC
rTTA 3G nucleic acid sequence
(SEQ ID NO: 6)
CCCCTCTCCCTCCCCCCCCCCTAACGTTACTGGCCGAAGCCGCTTGGAATAAGGCCGGTGTGCGTTTGTCTATAT
GTTATTTTCCACCATATTGCCGTCTTTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATGTCGTGAAGGAAGCAGTTCCTCTGGA
AGCTTCTTGAAGACAAACAACGTCTGTAGCGACCCTTTGCAGGCAGCGGAACCCCCCACCTGGCGACAGGTGCCT
CTGCGGCCAAAAGCCACGTGTATAAGATACACCTGCAAAGGCGGCACAACCCCAGTGCCACGTTGTGAGTTGGAT
AGTTGTGGAAAGAGTCAAATGGCTCTCCTCAAGCGTATTCAACAAGGGGCTGAAGGATGCCCAGAAGGTACCCCA
TTGTATGGGATCTGATCTGGGGCCTCGGTGCACATGCTTTACATGTGTTTAGTCGAGGTTAAAAAAACGTCTAGG
CCCCCCGAACCACGGGGACGTGGTTTTCCTTTGAAAAACACGATGATAAGCTTGCCACAACCATGGCTGAACAAG
ATGGATTGCACGCAGGTTCTCCGGCCGCTTGGGTGGAGAGGCTATTCGGCTATGACTGGGCACAACAGACAATCG
GCTGCTCTGATGCCGCCGTGTTCCGGCTGTCAGCGCAGGGGCGCCCGGTTCTTTTTGTCAAGACCGACCTGTCCG
GTGCCCTGAATGAACTGCAGGACGAGGCAGCGCGGCTATCGTGGCTGGCCACGACGGGCGTTCCTTGCGCAGCTG
TGCTCGACGTTGTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCTTGATCCGGCTA
CCTGCCCATTCGACCACCAAGCGAAACATCGCATCGAGCGAGCACGTACTCGGATGGAAGCCGGTCTTGTCGATC
AGGATGATCTGGACGAAGAGCATCAGGGGCTCGCGCCAGCCGAACTGTTCGCCAGGCTCAAGGCGCGCATGCCCG
ACGGCGAGGATCTCGTCGTGACCCATGGCGATGCCTGCTTGCCGAATATCATGGTGGAAAATGGCCGCTTTTCTG
GATTCATCGACTGTGGCCGGCTGGGTGTGGCGGACCGCTATCAGGACATAGCGTTGGCTACCCGTGATATTGCTG
AAGAGCTTGGCGGCGAATGGGCTGACCGCTTCCTCGTGCTTTACGGTATCGCCGCTCCCGATTCGCAGCGCATCG
CCTTCTATCGCCTTCTTGACGAGTTCTTCTGA
rTTA 3G amino acid sequence
(SEQ ID NO: 7)
MSRLDKSKVINSALELLNGVGIEGLTTRKLAQKLGVEQPTLYWHVKNKRALLDALPIEMLDRHHTHSCPLEGESW
QDFLRNNAKSYRCALLSHRDGAKVHLGTRPTEKQYETLENQLAFLCQQGFSLENALYALSAVGHFTLGCVLEEQE
HQVAKEERETPTTDSMPPLLKQAIELFDRQGAEPAFLFGLELIICGLEKQLKCESGGPTDALDDFDLDMLPADAL
DDFDLDMLPADALDDFDLDMLPG
Lentiviral Vector Sequence
(SEQ ID NO: 10)
TGGAAGGGCTAATTCACTCCCAAAGAAGACAAGATATCCTTGATCTGTGGATCTACCACACACAAGGCTACTTCC
CTGATTAGCAGAACTACACACCAGGGCCAGGGGTCAGATATCCACTGACCTTTGGATGGTGCTACAAGCTAGTAC
CAGTTGAGCCAGATAAGGTAGAAGAGGCCAATAAAGGAGAGAACACCAGCTTGTTACACCCTGTGAGCCTGCATG
GGATGGATGACCCGGAGAGAGAAGTGTTAGAGTGGAGGTTTGACAGCCGCCTAGCATTTCATCACGTGGCCCGAG
AGCTGCATCCGGAGTACTTCAAGAACTGCTGATATCGAGCTTGCTACAAGGGACTTTCCGCTGGGGACTTTCCAG
GGAGGCGTGGCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGCTTAAGCCT
CAATAAAGCTTGCCTTGAGTGCTTCAAGTAGTGTGTGCCCGTCTGTTGTGTGACTCTGGTAACTAGAGATCCCTC
AGACCCTTTTAGTCAGTGTGGAAAATCTCTAGCAGTGGCGCCCGAACAGGGACTTGAAAGCGAAAGGGAAACCAG
AGGAGCTCTCTCGACGCAGGACTCGGCTTGCTGAAGCGCGCACGGCAAGAGGCGAGGGGCGGCGACTGGTGAGTA
CGCCAAAAATTTTGACTAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTAAAACATATAGTATGGG
CAAGCAGGGAGCTAGAACGATTCGCAGTTAATCCTGGCCTGTTAGAAACATCAGAAGGCTGTAGACAAATACTGG
GACAGCTACAACCATCCCTTCAGACAGGATCAGAAGAACTTAGATCATTATATAATACAGTAGCAACCCTCTATT
GTGTGCATCAAAGGATAGAGATAAAAGACACCAAGGAAGCTTTAGACAAGATAGAGGAAGAGCAAAACAAAAGTA
AGACCACCGCACAGCAAGCGGCCGGCCGCTGATCTTCAGACCTGGAGGAGGAGATATGAGGGACAATTGGAGAAG
TGAATTATATAAATATAAAGTAGTAAAAATTGAACCATTAGGAGTAGCACCCACCAAGGCAAAGAGAAGAGTGGT
GCAGAGAGAAAAAAGAGCAGTGGGAATAGGAGCTTTGTTCCTTGGGTTCTTGGGAGCAGCAGGAAGCACTATGGG
CGCAGCGTCAATGACGCTGACGGTACAGGCCAGACAATTATTGTCTGGTATAGTGCAGCAGCAGAACAATTTGCT
GAGGGCTATTGAGGCGCAACAGCATCTGTTGCAACTCACAGTCTGGGGCATCAAGCAGCTCCAGGCAAGAATCCT
GGCTGTGGAAAGATACCTAAAGGATCAACAGCTCCTGGGGATTTGGGGTTGCTCTGGAAAACTCATTTGCACCAC
TGCTGTGCCTTGGAATGCTAGTTGGAGTAATAAATCTCTGGAACAGATTTGGAATCACACGACCTGGATGGAGTG
GGACAGAGAAATTAACAATTACACAAGCTTAATACACTCCTTAATTGAAGAATCGCAAAACCAGCAAGAAAAGAA
TGAACAAGAATTATTGGAATTAGATAAATGGGCAAGTTTGTGGAATTGGTTTAACATAACAAATTGGCTGTGGTA
TATAAAATTATTCATAATGATAGTAGGAGGCTTGGTAGGTTTAAGAATAGTTTTTGCTGTACTTTCTATAGTGAA
TAGAGTTAGGCAGGGATATTCACCATTATCGTTTCAGACCCACCTCCCAACCCCGAGGGGACCCGACAGGCCCGA
AGGAATAGAAGAAGAAGGTGGAGAGAGAGACAGAGACAGATCCATTCGATTAGTGAACGGATCTCGACGGTATCG
CCTTTAAAAGAAAAGGGGGGATTGGGGGGTACAGTGCAGGGGAAAGAATAGTAGACATAATAGCAACAGACATAC
AAACTAAAGAATTACAAAAACAAATTACAAAAATTCAAAATTTTCGGGTTTATTACAGGGACAGCTAAGATACAT
TGATGAGTTTGGACAAACCACAACTAGAATGCAGTGAAAAAAATGCTTTATTTGTGAAATTTGTGATGCTATTGC
TTTATTTGTAACCATTATAAGCTGCAATAAACAAGTTAGAGATCCAGTTTATTTCAGCGAGGGGGCAGGGCCTGC
ATGTGAAGGGCGTCGTAGGTGTCCTTGGTGGCTGTACTGAGACCCTGGTAAAGGCCATCGTGCCCCTTGCCCCTC
CGGCGCTCGCCTTTCATCCCAATCTCACTGTAGGCCTCCGCCATCTTATCTTTCTGCAGTTCATTGTACAGGCCT
TCCTGAGGGTTCTTCCTTCTCGGCTTTCCCCCCATCTCAGGGTCCCGGCCACGTCTCTTGTCCAAAACATCGTAC
TCCTCTCTTCGTCCTAGATTGAGCTCGTTATAGAGCTGGTTCTGGCCCTGCTTGTACGCGGGGGCGTCTGCGCTC
CTGCTGAACTTCACTCTCAGTTCACATCCTCCTTCTTCTTCTTCTGGAAATCGGCAGCTACAGCCATCTTCCTCT
TGAGTAGTTTGTACTGGTCTCATAAATGGTTGTTTGAATATATACAGGAGTTTCTTTCTGCCCCGTTTGCAGTAA
AGGGTGATAACCAGTGACAGGAGAAGGACCCCACAAGTCCCGGCCAAGGGCGCCCAGATGTAGATATCACAGGCG
AAGTCCAGCCCCCTCGTGTGCACTGCGCCCCCCGCCGCTGGCCGGCACGCCTCTGGGCGCAGGGACAGGGGCTGC
GACGCGATGGTGGGCGCCGGTGTTGGTGGTCGCGGCGCTGGCGTCGTGGTTCCGGACTTGATCTCCACCTTTGTG
CCCTGTCCAAAGGTCCAAGGCACCTCCTTGCTCTGCTGGCAGTAGTAGGTGGCGAAATCGTCAGGCTGCAGGCTG
GAGATGGTCAGGGTAAAGTCGGTGCCGCTGCCGCTGCCGGAAAACCTGCTAGGCACGCCGGAGCCCTGATTGCTG
GCGGCATAGATCAGCAGTTTGGGGGCCTTGCCGGGCTTCTGCTGGAACCAGTTCATGAAGCTGATGCCGTAATTG
TCCACGCTCTCGGAGGCCCTGCATGTGATGGTCACCCTGTCTCCCACGGAAGCGCTCAGGCTGCTAGGGCTCTGG
GTCATCTGAATGTCGGAGCCGCCGCCGCCGCTGCCGCCGCCGCCGGATCCGCCGCCACCAGAGCTCACTGTCACC
AGGGTGCCCTGGCCCCAGTAGTCCATAGCGGGCCTGCCCCTGGCGCAGTAGTAGACGGCTGTGTCCTCGGACCTC
AGGCTGCTGAGCTCCATGTAGGCGGTATTGGTGGACTCATCGGCGGTGATGGTGGCCTTGGACTTGAACTTCTGG
TTGTAGCCTGTGCCGCCGTTGTAAGGGTAGATGTAGCCGATCCACTCCAGTCCCTGGCCAGGGGCTTGCCTCACC
CAGTGCATGTTGTAGTCGGTGAAGGTGTAGCCGGAGGCCTTGCAGCTCACCTTCACGCTGCTGCCAGGTTTCTTC
ACCTCAGCGCCGCTCTGCACGAGCTGCACCTGAGGTCTGGCGGCGTGCAGCAGCAGGGCCAGAGGCAGCAGCAGA
GCTGTGACGGGCAGAGCCATGGTGGCTTACGAGGGTAGGAAGTGGTACGGAAAGTTGGTATAAGACAAAAGTGTT
GTGGAATTGCTCCAGGCGATCTGACGGTTCACTAAACGAGCTCTGCTTTTATAGGCGCCCACCGTACACGCCTAA
AGCTTATACGTTCTCTATCACTGATAGGGAGTAAACTGGATATACGTTCTCTATCACTGATAGGGAGTAAACTGT
AGATACGTTCTCTATCACTGATAGGGAGTAAACTGGTCATACGTTCTCTATCACTGATAGGGAGTAAACTCCTTA
TACGTTCTCTATCACTGATAGGGAGTAAAGTCTGCATACGTTCTCTATCACTGATAGGGAGTAAACTCTTCATAC
GTTCTCTATCACTGATAGGGAGTAAACTCCGATGGATCTATTTCCGGTGAATTCCGCTTTGGTTAATGGGAAGTG
GCTAATGGATAGAGGCTGAAAGAATTGGGAGGAGCAGACTAGCAAAAGTCTAGATTCCTGAAAACAGAGCATAAA
GGGCAATTCTGGTGAAGGCTCAGGGGGAAATGAGGAACAAGGTATTGAAAATCGAAGTAAATGCCATCCTTGTTG
TAAGTAGCAAAAACCTTGGCAAAATTGTGTCCTGTTCTAGAACTTTATGGAATATAAAAATTATGAGCCATTTGC
TAGTATATCTGCTGAAAGAAATATCTAAGAGGCAAAGCATTCAGGCTACTGTGTGACTACTTTTAGCCACCAGGA
AATTTAACCCAGCAAGAAGGGAGCCATGGGAATAGATTTTGCAAACCAGCACAGATGGTGACTCTACCTCCCTCT
GCTGTCCTGTCTCCCATAAGCCAAACTCTGTGCTGTGAGCTGTTAAAGTCCAAGAATTATTTCTGGGGAATCTGT
ACTCTTAATAATTTACAGACAACACAGGTCTGCTTTGGATCTGATCAGACAGACTAAATCTTGGGGACTGTACCA
GTGGCCACTGAGAAAAGGGGCTCGGAATGTTGTCTTGGATAGAAAAATATAATAAGAAACATTGGTGTGAATCTA
GCATCAGAAAGATGACGTGAGGACAGCAAGGAAGAGCTGGAAACTGAGGTTTACTCACAGGCTCCTCACCCTCCG
CTGATGGGCAGGTGTGTGAGCTCCAGCATGGAGCACCACAGCACTAGGTGGGAGAACGGTGATAGGGTGATGGGG
CAGCCTGTGAGCTGGGGCAGTGTAGGCAGAGGAGGAACTGTATCACCACAGAAACTTCTGCCTTCACACATCCCT
CCAGCTAGGCAGGACAGGTAGAGAGTCCAGTGTCCTGGAGCACTAGACCTAAGGAAGGCTGCATGGGGAGGACAA
AGGACAGTGACATCACAGGATACCCCTCCCATCAGGAAAATCAAGGCCCAGAACTCACTCGGCTCTTCCCCAGGA
GAACCTCGAGGCCACCATGTCTAGACTGGACAAGAGCAAAGTCATAAACTCTGCTCTGGAATTACTCAATGGAGT
CGGTATCGAAGGCCTGACGACAAGGAAACTCGCTCAAAAGCTGGGAGTTGAGCAGCCTACCCTGTACTGGCACGT
GAAGAACAAGCGGGCCCTGCTCGATGCCCTGCCAATCGAGATGCTGGACAGGCATCATACCCACTCCTGCCCCCT
GGAAGGCGAGTCATGGCAAGACTTTCTGCGGAACAACGCCAAGTCATACCGCTGTGCTCTCCTCTCACATCGCGA
CGGGGCTAAAGTGCATCTCGGCACCCGCCCAACAGAGAAACAGTACGAAACCCTGGAAAATCAGCTCGCGTTCCT
GTGTCAGCAAGGCTTCTCCCTGGAGAACGCACTGTACGCTCTGTCCGCCGTGGGCCACTTTACACTGGGCTGCGT
ATTGGAGGAACAGGAGCATCAAGTAGCAAAAGAGGAAAGAGAGACACCTACCACCGATTCTATGCCCCCACTTCT
GAAACAAGCAATTGAGCTGTTCGACCGGCAGGGAGCCGAACCTGCCTTCCTTTTCGGCCTGGAACTAATCATATG
TGGCCTGGAGAAACAGCTAAAGTGCGAAAGCGGCGGGCCGACCGACGCCCTTGACGATTTTGACTTAGACATGCT
CCCAGCCGATGCCCTTGACGACTTTGACCTTGATATGCTGCCTGCTGACGCTCTTGACGATTTTGACCTTGACAT
GCTCCCCGGGTAAGGATCCCGACGCGTCTGGAACAATCAACCTCTGGATTACAAAATTTGTGAAAGATTGACTGG
TATTCTTAACTATGTTGCTCCTTTTACGCTATGTGGATACGCTGCTTTAATGCCTTTGTATCATGCTATTGCTTC
CAGGCAACGTGGCGTGGTGTGCACTGTGTTTGCTGACGCAACCCCCACTGGTTGGGGCATTGCCACCACCTGTCA
GCTCCTTTCCGGGACTTTCGCTTTCCCCCTCCCTATTGCCACGGCGGAACTCATCGCCGCCTGCCTTGCCCGCTG
CTGGACAGGGGCTCGGCTGTTGGGCACTGACAATTCCGTGGTGTTGTCGGGGAAGCTGACGTCCTTTCCATGGCT
GCTCGCCTGTGTTGCCACCTGGATTCTGCGCGGGACGTCCTTCTGCTACGTCCCTTCGGCCCTCAATCCAGCGGA
CCTTCCTTCCCGCGGCCTGCTGCCGGCTCTGCGGCCTCTTCCGCGTCTTCGCCTTCGCCCTCAGACGAGTCGGAT
CTCCCTTTGGGCCGCCTCCCCGCCTGGAATTAATTCTGCAGTCGAGACCTAGAAAAACATGGAGCAATCACAAGT
AGCAATACAGCAGCTACCAATGCTGATTGTGCCTGGCTAGAAGCACAAGAGGAGGAGGAGGTGGGTTTTCCAGTC
ACACCTCAGGTACCTTTAAGACCAATGACTTACAAGGCAGCTGTAGATCTTAGCCACTTTTTAAAAGAAAAGAGG
GGACTGGAAGGGCTAATTCACTCCCAACGAAGACAAGATATCCTTGATCTGTGGATCTACCACACACAAGGCTAC
TTCCCTGATTAGCAGAACTACACACCAGGGCCAGGGGTCAGATATCCACTGACCTTTGGATGGTGCTACAAGCTA
GTACCAGTTGAGCCAGATAAGGTAGAAGAGGCCAATAAAGGAGAGAACACCAGCTTGTTACACCCTGTGAGCCTG
CATGGGATGGATGACCCGGAGAGAGAAGTGTTAGAGTGGAGGTTTGACAGCCGCCTAGCATTTCATCACGTGGCC
CGAGAGCTGCATCCGGAGTACTTCAAGAACTGCTGATATCGAGCTTGCTACAAGGGACTTTCCGCTGGGGACTTT
CCAGGGAGGCGTGGCCTGGGCGGGACTGGGGAGTGGCGAGCCCTCAGATCCTGCATATAAGCAGCTGCTTTTTGC
CTGTACTGGGTCTCTCTGGTTAGACCAGATCTGAGCCTGGGAGCTCTCTGGCTAACTAGGGAACCCACTGCTTAA
GCCTCAATAAAGCTTGCCTTGAGTGCTTCAAGTAGTGTGTGCCCGTCTGTTGTGTGACTCTGGTAACTAGAGATC
CCTCAGACCCTTTTAGTCAGTGTGGAAAATCTCTAGCAGT

Annotation of certain features within the vector sequence above:

| Feature | Nucleotide position numbers |
|---|---|
| 5′ LTR | 1 . . . 635 |
| -psi (Packaging signal) | 685 . . . 822 |
| RRE | 1303 . . . 1536 |
| cPPT/CTS | 2028 . . . 2151 |
| SV40 poly(A) signal | 2166 . . . 2287 (reverse orientation) |
| STOP codon | 2304 . . . 2306 (reverse orientation) |
| anti-CD33 CAR | 2307 . . . 3770 (reverse orientation) |
| Kozak sequence | 3771 . . . 3776 (reverse orientation) |
| TRE3G promoter | 3777 . . . 4154 (reverse orientation) |
| pCD8 | 4181 . . . 5180 |
| Tet-On 3G rTTA | 5192 . . . 5938 |
| WPRE | 5960 . . . 6551 |
| 3′ LTR | 6754 . . . 7390 |

The lentiviral vector is cloned into a cloning plasmid, e.g., pUC19, for amplification in bacteria. Amplification, purification of the lentiviral fragment, lentiviral packaging and concentration, and transduction of target cells is performed according to standard methods.

Normal CD4+/CD8+ T cells are obtained and cultured using standard protocols. T cells are transduced with lentiviral supernatant 24 hours following stimulation at a MOI of 1-5. T-cells are grown in T-cell media for up to 15 days, and then cryopreserved.

HSCs are obtained from bone marrow samples selected for CD34 and cryopreserved. HSCs are transduced with lentiviral supernatant 24 hours after thawing according to standard protocols.

The MOLM14 cell line, expressing CD33, is cultured in R10 media (RPMI media, 10% fetal calf serum, penicillin, and streptomycin).

Surface expression of anti-CD33 CAR is detected by using a labeled ligand (CD33 extracellular domain) or antibody specifically binding the anti-CD33 CAR.

Cytotoxicity assays and cytokine excretion assays are performed using standard protocols. CD33 expressing cells, e.g., MOLM cells expressing luciferase, are incubated at different ratios with effector T cells. Killing is monitored and calculated according to standard protocols. Cytokine assays are performed according to standard protocols.

HSC cells are differentiated into pre-T-cells and mature T-cells according to standard protocols. Expression of TCRb and anti-CD33 CAR is monitored during differentiation.

Mature T cells as well as pre-T cells transduced with the lentiviral vector demonstrate inducible expression of the anti-CD33 CAR, which can be induced by administration of doxycycline to the cells, and which is abolished upon withdrawal of doxycycline from the cells.

HSCs transduced with the lentiviral vector do not exhibit inducible expression of the anti-CD33 CAR, even in the presence of doxycycline. However, pre-T cells and mature T-cells differentiated from these HSCs show inducible expression of the anti-CD33 CAR upon administration of doxycycline, which is abolished upon withdrawal of doxycycline from the cells.

When administered to a suitable mouse model, the transduced HSCs engraft and populate the hematopoietic niche. After a time sufficient for mature T-lymphocytes derived from these HSCs to emerge, anti-CD33 CAR expression can be induced in these mice by injection of doxycycline.

### Example 3: NK-Cell Specific Expression of a CD33 CAR

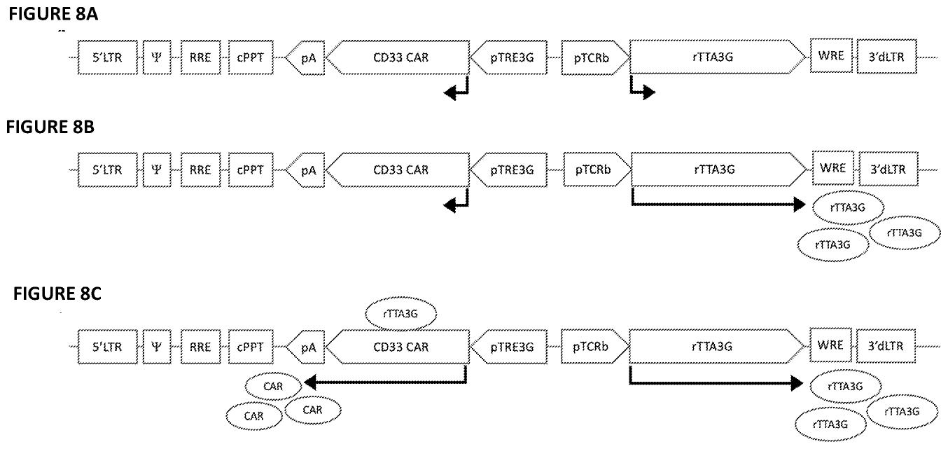

A lentiviral expression vector for inducible expression of a CD33 CAR in T-lymphocytes was designed. A schematic of the expression construct is provided in FIGS. 8A-8C.

Briefly, the lentiviral vector comprises a pLVX vector backbone (Takara Bio USA), an expression cassette comprising a nucleic acid sequence encoding a tet-responsive transactivator (rTTA 3G) under the control of a KLRK1 promoter (pKLRK1), and an expression cassette comprising a nucleic acid sequence encoding a CD33 CAR under the control of a tet-responsive promoter (pTRE). The expression cassette comprising the nucleic acid sequence encoding a CD33 CAR under the control of a tet-responsive promoter (pTRE) is oriented in a manner in which the direction of transcription of the CD33 CAR-encoding sequence is towards the 5′ LTR of the lentiviral vector, whereas the expression cassette comprising the nucleic acid sequence encoding the rTTA 3G under the control of the KLRK1 promoter is oriented in a manner in which the direction of transcription of the rTTA 3G-encoding sequence is towards the 3′ LTR of the lentiviral vector. The orientation of the CD33 CAR expression cassette towards the 5′LTR of the viral backbone allows for the use of a directional polyadenylation signal (pA), which does not interfere with transcription of the viral genome during packaging, but efficiently mediates polyadenylation and thus stabilization of the CD33 CAR transcript driven by the pTRE promoter. The backbone further comprises additional lentiviral elements, e.g., packaging signal (psi, or Ψ), Rev-response element (RRE), central polypurine tract/central termination sequence (is cloned into the pLVX-Tet3G vector (Clontech Laboratories/Takara Bio USA).

The sequences used in the vector design are provided below:

pTRE3G nucleic acid sequence
(SEQ ID NO: 1)
GAGTTTACTCCCTATCAGTGATAGAGAACGTATGAAGAGTTTACTCCCTATCAGTGATAGAGAACGTATGCAGAC
TTTACTCCCTATCAGTGATAGAGAACGTATAAGGAGTTTACTCCCTATCAGTGATAGAGAACGTATGACCAGTTT
ACTCCCTATCAGTGATAGAGAACGTATCTACAGTTTACTCCCTATCAGTGATAGAGAACGTATATCCAGTTTACT
CCCTATCAGTGATAGAGAACGTATAAGCTTTAGGCGTGTACGGTGGGCGCCTATAAAAGCAGAGCTCGTTTAGTG
AACCGTCAGATCGCCTGGAGCAATTCCACAACACTTTTGTCTTATACCAACTTTCCGTACCACTTCCTACCCTCG
TAAA
Kozak sequence
GCCACC
Anti-CD33 CAR-encoding nucleotide sequence
(SEQ ID NO: 2)
ATGGCTCTGCCCGTCACAGCTCTGCTGCTGCCTCTGGCCCTGCTGCTGCACGCCGCCAGACCTCAGGTGCAGCTC
GTGCAGAGCGGCGCTGAGGTGAAGAAACCTGGCAGCAGCGTGAAGGTGAGCTGCAAGGCCTCCGGCTACACCTTC
ACCGACTACAACATGCACTGGGTGAGGCAAGCCCCTGGCCAGGGACTGGAGTGGATCGGCTACATCTACCCTTAC
AACGGCGGCACAGGCTACAACCAGAAGTTCAAGTCCAAGGCCACCATCACCGCCGATGAGTCCACCAATACCGCC
TACATGGAGCTCAGCAGCCTGAGGTCCGAGGACACAGCCGTCTACTACTGCGCCAGGGGCAGGCCCGCTATGGAC
TACTGGGGCCAGGGCACCCTGGTGACAGTGAGCTCTGGTGGCGGCGGATCCGGCGGCGGCGGCAGCGGCGGCGGC
GGCTCCGACATTCAGATGACCCAGAGCCCTAGCAGCCTGAGCGCTTCCGTGGGAGACAGGGTGACCATCACATGC
AGGGCCTCCGAGAGCGTGGACAATTACGGCATCAGCTTCATGAACTGGTTCCAGCAGAAGCCCGGCAAGGCCCCC
AAACTGCTGATCTATGCCGCCAGCAATCAGGGCTCCGGCGTGCCTAGCAGGTTTTCCGGCAGCGGCAGCGGCACC
GACTTTACCCTGACCATCTCCAGCCTGCAGCCTGACGATTTCGCCACCTACTACTGCCAGCAGAGCAAGGAGGTG
CCTTGGACCTTTGGACAGGGCACAAAGGTGGAGATCAAGTCCGGAACCACGACGCCAGCGCCGCGACCACCAACA
CCGGCGCCCACCATCGCGTCGCAGCCCCTGTCCCTGCGCCCAGAGGCGTGCCGGCCAGCGGCGGGGGGCGCAGTG
CACACGAGGGGGCTGGACTTCGCCTGTGATATCTACATCTGGGCGCCCTTGGCCGGGACTTGTGGGGTCCTTCTC
CTGTCACTGGTTATCACCCTTTACTGCAAACGGGGCAGAAAGAAACTCCTGTATATATTCAAACAACCATTTATG
AGACCAGTACAAACTACTCAAGAGGAAGATGGCTGTAGCTGCCGATTTCCAGAAGAAGAAGAAGGAGGATGTGAA
CTGAGAGTGAAGTTCAGCAGGAGCGCAGACGCCCCCGCGTACAAGCAGGGCCAGAACCAGCTCTATAACGAGCTC
AATCTAGGACGAAGAGAGGAGTACGATGTTTTGGACAAGAGACGTGGCCGGGACCCTGAGATGGGGGGAAAGCCG
AGAAGGAAGAACCCTCAGGAAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTCTCAGTACAGCCACCAAGGAC
ACCTACGACGCCCTTCACATGCAGGCCCTGCCCCCTCGCTGA
Anti-CD33 CAR amino acid sequence
(SEQ ID NO: 3)
MALPVTALLLPLALLLHAARPQVQLVQSGAEVKKPGSSVKVSCKASGYTFTDYNMHWVRQAPGQGLEWIGYIYPY
NGGTGYNQKFKSKATITADESTNTAYMELSSLRSEDTAVYYCARGRPAMDYWGQGTLVTVSSGGGGSGGGGSGGG
GSDIQMTQSPSSLSASVGDRVTITCRASESVDNYGISFMNWFQQKPGKAPKLLIYAASNQGSGVPSRFSGSGSGT
DFTLTISSLQPDDFATYYCQQSKEVPWTFGQGTKVEIKSGTTTPAPRPPTPAPTIASQPLSLRPEACRPAAGGAV
HTRGLDFACDIYIWAPLAGTCGVLLLSLVITLYCKRGRKKLLYIFKQPFMRPVQTTQEEDGCSCRFPEEEEGGCE
LRVKFSRSADAPAYKQGQNQLYNELNLGRREEYDVLDKRRGRDPEMGGKPRRKNPQEGLYNELQKDKMAEAYSEI
GMKGERRRGKGHDGLYQGLSTATKDTYDALHMQALPPR
SV40 polyA signal
(SEQ ID NO: 4)
AACTTGTTTATTGCAGCTTATAATGGTTACAAATAAAGCAATAGCATCACAAATTTCACAAATAAAGCATTTTTT
TCACTGCATTCTAGTTGTGGTTTGTCCAAACTCATCAATGTATCTTA
KLRK1 promoter sequence
(SEQ ID NO: 11)
TGTTCTCAAATGACTTGAGCCCAGGCTGTTTGCAAAATATTGTAATTTTTCTTCTAACAATAGTTTTTGAAAGTT
ATATAGATACGTCTATATGAAGTTTGTTCAGGTACTTTTATAGTCAAATTATTCTTTCTGCAGAAGTTAATTGCG
TTGGTTTTATTTCACTTATTAGAAAGTATTTTATCTGAAAACTATGCTATGTCCTTTTGCAATTCACATAGAATA
AACCAGTACAAATGCCCAGCAAATCCCCAATAAGTTTACTATCATGACTACAGCAAAGTGAGTGTATGTTCAACA
TTGAGAAAGAAATTGGAGTTTTAACATACTATTTCTTTATCTAACTAATTGTTTTAACCCGACATACAGGACCCA
TTTTAGAATTTTCTTCTTTAAGAATCCTTTCCACACTCTGTCAGTTGGTTTAGGGACCTTTATATATGACAATAA
AACTCTCCCTGGTGCTTAGCTTTATCATGGTATTTATTTCAGAAATCTTCTTTTAG
rTTA 3G nucleic acid sequence
(SEQ ID NO: 6)
CCCCTCTCCCTCCCCCCCCCCTAACGTTACTGGCCGAAGCCGCTTGGAATAAGGCCGGTGTGCGTTTGTCTATAT
GTTATTTTCCACCATATTGCCGTCTTTTGGCAATGTGAGGGCCCGGAAACCTGGCCCTGTCTTCTTGACGAGCAT
TCCTAGGGGTCTTTCCCCTCTCGCCAAAGGAATGCAAGGTCTGTTGAATGTCGTGAAGGAAGCAGTTCCTCTGGA
AGCTTCTTGAAGACAAACAACGTCTGTAGCGACCCTTTGCAGGCAGCGGAACCCCCCACCTGGCGACAGGTGCCT
CTGCGGCCAAAAGCCACGTGTATAAGATACACCTGCAAAGGCGGCACAACCCCAGTGCCACGTTGTGAGTTGGAT
AGTTGTGGAAAGAGTCAAATGGCTCTCCTCAAGCGTATTCAACAAGGGGCTGAAGGATGCCCAGAAGGTACCCCA
TTGTATGGGATCTGATCTGGGGCCTCGGTGCACATGCTTTACATGTGTTTAGTCGAGGTTAAAAAAACGTCTAGG
CCCCCCGAACCACGGGGACGTGGTTTTCCTTTGAAAAACACGATGATAAGCTTGCCACAACCATGGCTGAACAAG
ATGGATTGCACGCAGGTTCTCCGGCCGCTTGGGTGGAGAGGCTATTCGGCTATGACTGGGCACAACAGACAATCG
GCTGCTCTGATGCCGCCGTGTTCCGGCTGTCAGCGCAGGGGCGCCCGGTTCTTTTTGTCAAGACCGACCTGTCCG
GTGCCCTGAATGAACTGCAGGACGAGGCAGCGCGGCTATCGTGGCTGGCCACGACGGGCGTTCCTTGCGCAGCTG
TGCTCGACGTTGTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCTTGATCCGGCTA
CCTGCCCATTCGACCACCAAGCGAAACATCGCATCGAGCGAGCACGTACTCGGATGGAAGCCGGTCTTGTCGATC
AGGATGATCTGGACGAAGAGCATCAGGGGCTCGCGCCAGCCGAACTGTTCGCCAGGCTCAAGGCGCGCATGCCCG
ACGGCGAGGATCTCGTCGTGACCCATGGCGATGCCTGCTTGCCGAATATCATGGTGGAAAATGGCCGCTTTTCTG
GATTCATCGACTGTGGCCGGCTGGGTGTGGCGGACCGCTATCAGGACATAGCGTTGGCTACCCGTGATATTGCTG
AAGAGCTTGGCGGCGAATGGGCTGACCGCTTCCTCGTGCTTTACGGTATCGCCGCTCCCGATTCGCAGCGCATCG
CCTTCTATCGCCTTCTTGACGAGTTCTTCTGA
rTTA 3G amino acid sequence
(SEQ ID NO: 7)
MSRLDKSKVINSALELLNGVGIEGLTTRKLAQKLGVEQPTLYWHVKNKRALLDALPIEMLDRHHTHSCPLEGESW
QDFLRNNAKSYRCALLSHRDGAKVHLGTRPTEKQYETLENQLAFLCQQGFSLENALYALSAVGHFTLGCVLEEQE
HQVAKEERETPTTDSMPPLLKQAIELFDRQGAEPAFLFGLELIICGLEKQLKCESGGPTDALDDFDLDMLPADAL
DDFDLDMLPADALDDFDLDMLPG
Lentiviral Vector Sequence
(SEQ ID NO: 12)
TGGAAGGGCTAATTCACTCCCAAAGAAGACAAGATATCCTTGATCTGTGGATCTACCACACACAAGGCTACTTCC
CTGATTAGCAGAACTACACACCAGGGCCAGGGGTCAGATATCCACTGACCTTTGGATGGTGCTACAAGCTAGTAC
CAGTTGAGCCAGATAAGGTAGAAGAGGCCAATAAAGGAGAGAACACCAGCTTGTTACACCCTGTGAGCCTGCATG
GGATGGATGACCCGGAGAGAGAAGTGTTAGAGTGGAGGTTTGACAGCCGCCTAGCATTTCATCACGTGGCCCGAG
AGCTGCATCCGGAGTACTTCAAGAACTGCTGATATCGAGCTTGCTACAAGGGACTTTCCGCTGGGGACTTTCCAG
GGAGGCGTGGCCTGGGCGGGACTGGGGAGTGGCGAGCCCTCAGATCCTGCATATAAGCAGCTGCTTTTTGCCTGT
ACTGGGTCTCTCTGGTTAGACCAGATCTGAGCCTGGGAGCTCTCTGGCTAACTAGGGAACCCACTGCTTAAGCCT
CAATAAAGCTTGCCTTGAGTGCTTCAAGTAGTGTGTGCCCGTCTGTTGTGTGACTCTGGTAACTAGAGATCCCTC
AGACCCTTTTAGTCAGTGTGGAAAATCTCTAGCAGTGGCGCCCGAACAGGGACTTGAAAGCGAAAGGGAAACCAG
AGGAGCTCTCTCGACGCAGGACTCGGCTTGCTGAAGCGCGCACGGCAAGAGGCGAGGGGCGGCGACTGGTGAGTA
CGCCAAAAATTTTGACTAGCGGAGGCTAGAAGGAGAGAGATGGGTGCGAGAGCGTCAGTATTAAGCGGGGGAGAA
TTAGATCGCGATGGGAAAAAATTCGGTTAAGGCCAGGGGGAAAGAAAAAATATAAATTAAAACATATAGTATGGG
CAAGCAGGGAGCTAGAACGATTCGCAGTTAATCCTGGCCTGTTAGAAACATCAGAAGGCTGTAGACAAATACTGG
GACAGCTACAACCATCCCTTCAGACAGGATCAGAAGAACTTAGATCATTATATAATACAGTAGCAACCCTCTATT
GTGTGCATCAAAGGATAGAGATAAAAGACACCAAGGAAGCTTTAGACAAGATAGAGGAAGAGCAAAACAAAAGTA
AGACCACCGCACAGCAAGCGGCCGGCCGCTGATCTTCAGACCTGGAGGAGGAGATATGAGGGACAATTGGAGAAG
TGAATTATATAAATATAAAGTAGTAAAAATTGAACCATTAGGAGTAGCACCCACCAAGGCAAAGAGAAGAGTGGT
GCAGAGAGAAAAAAGAGCAGTGGGAATAGGAGCTTTGTTCCTTGGGTTCTTGGGAGCAGCAGGAAGCACTATGGG
CGCAGCGTCAATGACGCTGACGGTACAGGCCAGACAATTATTGTCTGGTATAGTGCAGCAGCAGAACAATTTGCT
GAGGGCTATTGAGGCGCAACAGCATCTGTTGCAACTCACAGTCTGGGGCATCAAGCAGCTCCAGGCAAGAATCCT
GGCTGTGGAAAGATACCTAAAGGATCAACAGCTCCTGGGGATTTGGGGTTGCTCTGGAAAACTCATTTGCACCAC
TGCTGTGCCTTGGAATGCTAGTTGGAGTAATAAATCTCTGGAACAGATTTGGAATCACACGACCTGGATGGAGTG
GGACAGAGAAATTAACAATTACACAAGCTTAATACACTCCTTAATTGAAGAATCGCAAAACCAGCAAGAAAAGAA
TGAACAAGAATTATTGGAATTAGATAAATGGGCAAGTTTGTGGAATTGGTTTAACATAACAAATTGGCTGTGGTA
TATAAAATTATTCATAATGATAGTAGGAGGCTTGGTAGGTTTAAGAATAGTTTTTGCTGTACTTTCTATAGTGAA
TAGAGTTAGGCAGGGATATTCACCATTATCGTTTCAGACCCACCTCCCAACCCCGAGGGGACCCGACAGGCCCGA
AGGAATAGAAGAAGAAGGTGGAGAGAGAGACAGAGACAGATCCATTCGATTAGTGAACGGATCTCGACGGTATCG
CCTTTAAAAGAAAAGGGGGGATTGGGGGGTACAGTGCAGGGGAAAGAATAGTAGACATAATAGCAACAGACATAC
AAACTAAAGAATTACAAAAACAAATTACAAAAATTCAAAATTTTCGGGTTTATTACAGGGACAGCTAAGATACAT
TGATGAGTTTGGACAAACCACAACTAGAATGCAGTGAAAAAAATGCTTTATTTGTGAAATTTGTGATGCTATTGC
TTTATTTGTAACCATTATAAGCTGCAATAAACAAGTTAGAGATCCAGTTTATTTCAGCGAGGGGGCAGGGCCTGC
ATGTGAAGGGCGTCGTAGGTGTCCTTGGTGGCTGTACTGAGACCCTGGTAAAGGCCATCGTGCCCCTTGCCCCTC
CGGCGCTCGCCTTTCATCCCAATCTCACTGTAGGCCTCCGCCATCTTATCTTTCTGCAGTTCATTGTACAGGCCT
TCCTGAGGGTTCTTCCTTCTCGGCTTTCCCCCCATCTCAGGGTCCCGGCCACGTCTCTTGTCCAAAACATCGTAC
TCCTCTCTTCGTCCTAGATTGAGCTCGTTATAGAGCTGGTTCTGGCCCTGCTTGTACGCGGGGGCGTCTGCGCTC
CTGCTGAACTTCACTCTCAGTTCACATCCTCCTTCTTCTTCTTCTGGAAATCGGCAGCTACAGCCATCTTCCTCT
TGAGTAGTTTGTACTGGTCTCATAAATGGTTGTTTGAATATATACAGGAGTTTCTTTCTGCCCCGTTTGCAGTAA
AGGGTGATAACCAGTGACAGGAGAAGGACCCCACAAGTCCCGGCCAAGGGCGCCCAGATGTAGATATCACAGGCG
AAGTCCAGCCCCCTCGTGTGCACTGCGCCCCCCGCCGCTGGCCGGCACGCCTCTGGGCGCAGGGACAGGGGCTGC
GACGCGATGGTGGGCGCCGGTGTTGGTGGTCGCGGCGCTGGCGTCGTGGTTCCGGACTTGATCTCCACCTTTGTG
CCCTGTCCAAAGGTCCAAGGCACCTCCTTGCTCTGCTGGCAGTAGTAGGTGGCGAAATCGTCAGGCTGCAGGCTG
GAGATGGTCAGGGTAAAGTCGGTGCCGCTGCCGCTGCCGGAAAACCTGCTAGGCACGCCGGAGCCCTGATTGCTG
GCGGCATAGATCAGCAGTTTGGGGGCCTTGCCGGGCTTCTGCTGGAACCAGTTCATGAAGCTGATGCCGTAATTG
TCCACGCTCTCGGAGGCCCTGCATGTGATGGTCACCCTGTCTCCCACGGAAGCGCTCAGGCTGCTAGGGCTCTGG
GTCATCTGAATGTCGGAGCCGCCGCCGCCGCTGCCGCCGCCGCCGGATCCGCCGCCACCAGAGCTCACTGTCACC
AGGGTGCCCTGGCCCCAGTAGTCCATAGCGGGCCTGCCCCTGGCGCAGTAGTAGACGGCTGTGTCCTCGGACCTC
AGGCTGCTGAGCTCCATGTAGGCGGTATTGGTGGACTCATCGGCGGTGATGGTGGCCTTGGACTTGAACTTCTGG
TTGTAGCCTGTGCCGCCGTTGTAAGGGTAGATGTAGCCGATCCACTCCAGTCCCTGGCCAGGGGCTTGCCTCACC
CAGTGCATGTTGTAGTCGGTGAAGGTGTAGCCGGAGGCCTTGCAGCTCACCTTCACGCTGCTGCCAGGTTTCTTC
ACCTCAGCGCCGCTCTGCACGAGCTGCACCTGAGGTCTGGCGGCGTGCAGCAGCAGGGCCAGAGGCAGCAGCAGA
GCTGTGACGGGCAGAGCCATGGTGGCTTACGAGGGTAGGAAGTGGTACGGAAAGTTGGTATAAGACAAAAGTGTT
GTGGAATTGCTCCAGGCGATCTGACGGTTCACTAAACGAGCTCTGCTTTTATAGGCGCCCACCGTACACGCCTAA
AGCTTATACGTTCTCTATCACTGATAGGGAGTAAACTGGATATACGTTCTCTATCACTGATAGGGAGTAAACTGT
AGATACGTTCTCTATCACTGATAGGGAGTAAACTGGTCATACGTTCTCTATCACTGATAGGGAGTAAACTCCTTA
TACGTTCTCTATCACTGATAGGGAGTAAAGTCTGCATACGTTCTCTATCACTGATAGGGAGTAAACTCTTCATAC
GTTCTCTATCACTGATAGGGAGTAAACTCCGATGGATCTATTTCCGGTGAATTCCTGTTCTCAAATGACTTGAGC
CCAGGCTGTTTGCAAAATATTGTAATTTTTCTTCTAACAATAGTTTTTGAAAGTTATATAGATACGTCTATATGA
AGTTTGTTCAGGTACTTTTATAGTCAAATTATTCTTTCTGCAGAAGTTAATTGCGTTGGTTTTATTTCACTTATT
AGAAAGTATTTTATCTGAAAACTATGCTATGTCCTTTTGCAATTCACATAGAATAAACCAGTACAAATGCCCAGC
AAATCCCCAATAAGTTTACTATCATGACTACAGCAAAGTGAGTGTATGTTCAACATTGAGAAAGAAATTGGAGTT
TTAACATACTATTTCTTTATCTAACTAATTGTTTTAACCCGACATACAGGACCCATTTTAGAATTTTCTTCTTTA
AGAATCCTTTCCACACTCTGTCAGTTGGTTTAGGGACCTTTATATATGACAATAAAACTCTCCCTGGTGCTTAGC
TTTATCATGGTATTTATTTCAGAAATCTTCTTTTAGTCGAGGCCACCATGTCTAGACTGGACAAGAGCAAAGTCA
TAAACTCTGCTCTGGAATTACTCAATGGAGTCGGTATCGAAGGCCTGACGACAAGGAAACTCGCTCAAAAGCTGG
GAGTTGAGCAGCCTACCCTGTACTGGCACGTGAAGAACAAGCGGGCCCTGCTCGATGCCCTGCCAATCGAGATGC
TGGACAGGCATCATACCCACTCCTGCCCCCTGGAAGGCGAGTCATGGCAAGACTTTCTGCGGAACAACGCCAAGT
CATACCGCTGTGCTCTCCTCTCACATCGCGACGGGGCTAAAGTGCATCTCGGCACCCGCCCAACAGAGAAACAGT
ACGAAACCCTGGAAAATCAGCTCGCGTTCCTGTGTCAGCAAGGCTTCTCCCTGGAGAACGCACTGTACGCTCTGT
CCGCCGTGGGCCACTTTACACTGGGCTGCGTATTGGAGGAACAGGAGCATCAAGTAGCAAAAGAGGAAAGAGAGA
CACCTACCACCGATTCTATGCCCCCACTTCTGAAACAAGCAATTGAGCTGTTCGACCGGCAGGGAGCCGAACCTG
CCTTCCTTTTCGGCCTGGAACTAATCATATGTGGCCTGGAGAAACAGCTAAAGTGCGAAAGCGGCGGGCCGACCG
ACGCCCTTGACGATTTTGACTTAGACATGCTCCCAGCCGATGCCCTTGACGACTTTGACCTTGATATGCTGCCTG
CTGACGCTCTTGACGATTTTGACCTTGACATGCTCCCCGGGTAAGGATCCCGACGCGTCTGGAACAATCAACCTC
TGGATTACAAAATTTGTGAAAGATTGACTGGTATTCTTAACTATGTTGCTCCTTTTACGCTATGTGGATACGCTG
CTTTAATGCCTTTGTATCATGCTATTGCTTCCCGTATGGCTTTCATTTTCTCCTCCTTGTATAAATCCTGGTTGC
TGTCTCTTTATGAGGAGTTGTGGCCCGTTGTCAGGCAACGTGGCGTGGTGTGCACTGTGTTTGCTGACGCAACCC
CCACTGGTTGGGGCATTGCCACCACCTGTCAGCTCCTTTCCGGGACTTTCGCTTTCCCCCTCCCTATTGCCACGG
CGGAACTCATCGCCGCCTGCCTTGCCCGCTGCTGGACAGGGGCTCGGCTGTTGGGCACTGACAATTCCGTGGTGT
TGTCGGGGAAGCTGACGTCCTTTCCATGGCTGCTCGCCTGTGTTGCCACCTGGATTCTGCGCGGGACGTCCTTCT
GCTACGTCCCTTCGGCCCTCAATCCAGCGGACCTTCCTTCCCGCGGCCTGCTGCCGGCTCTGCGGCCTCTTCCGC
GTCTTCGCCTTCGCCCTCAGACGAGTCGGATCTCCCTTTGGGCCGCCTCCCCGCCTGGAATTAATTCTGCAGTCG
AGACCTAGAAAAACATGGAGCAATCACAAGTAGCAATACAGCAGCTACCAATGCTGATTGTGCCTGGCTAGAAGC
ACAAGAGGAGGAGGAGGTGGGTTTTCCAGTCACACCTCAGGTACCTTTAAGACCAATGACTTACAAGGCAGCTGT
AGATCTTAGCCACTTTTTAAAAGAAAAGAGGGGACTGGAAGGGCTAATTCACTCCCAACGAAGACAAGATATCCT
TGATCTGTGGATCTACCACACACAAGGCTACTTCCCTGATTAGCAGAACTACACACCAGGGCCAGGGGTCAGATA
TCCACTGACCTTTGGATGGTGCTACAAGCTAGTACCAGTTGAGCCAGATAAGGTAGAAGAGGCCAATAAAGGAGA
GAACACCAGCTTGTTACACCCTGTGAGCCTGCATGGGATGGATGACCCGGAGAGAGAAGTGTTAGAGTGGAGGTT
TGACAGCCGCCTAGCATTTCATCACGTGGCCCGAGAGCTGCATCCGGAGTACTTCAAGAACTGCTGATATCGAGC
TTGCTACAAGGGACTTTCCGCTGGGGACTTTCCAGGGAGGCGTGGCCTGGGCGGGACTGGGGAGTGGCGAGCCCT
CAGATCCTGCATATAAGCAGCTGCTTTTTGCCTGTACTGGGTCTCTCTGGTTAGACCAGATCTGAGCCTGGGAGC
TCTCTGGCTAACTAGGGAACCCACTGCTTAAGCCTCAATAAAGCTTGCCTTGAGTGCTTCAAGTAGTGTGTGCCC
GTCTGTTGTGTGACTCTGGTAACTAGAGATCCCTCAGACCCTTTTAGTCAGTGTGGAAAATCTCTAGCAGT

Annotation of certain features within the vector sequence above:

| Feature | Nucleotide position numbers |
|---|---|
| 5′ LTR | 1 . . . 635 |
| -psi (Packaging signal) | 685 . . . 822 |
| RRE | 1303 . . . 1536 |
| cPPT/CTS | 2028 . . . 2151 |
| SV40 poly(A) signal | 2166 . . . 2287 |
| STOP codon | 2304 . . . 2306 |
| anti-CD33 CAR | 2307 . . . 3770 |
| Kozak sequence | 3771 . . . 3776 |
| TRE3G promoter | 3777 . . . 4154 |
| pKLRK1 | 4181 . . . 4686 |
| Tet-On 3G rTTA | 4698 . . . 5444 |
| WPRE | 5466 . . . 6057 |
| 3′ LTR | 6260 . . . 6896 |

The lentiviral vector is cloned into a cloning plasmid, e.g., pUC19, for amplification in bacteria. Amplification, purification of the lentiviral fragment, lentiviral packaging and concentration, and transduction of target cells is performed according to standard methods.

Normal CD3-CD56+CD7+CD127-NKp46+NK cells are obtained and cultured using standard protocols. NK cells are transduced with lentiviral supernatant 24 hours following stimulation at a MOI of 1-5. NK cells are expanded for up to 15 days, and then cryopreserved.

HSCs are obtained from bone marrow samples selected for CD34+ and cryopreserved. HSCs are transduced with lentiviral supernatant 24 hours after thawing according to standard protocols.

The MOLM14 cell line, expressing CD33, is cultured in R10 media (RPMI media, 10% fetal calf serum, penicillin, and streptomycin).

Surface expression of anti-CD33 CAR is detected by using a labeled ligand (CD33 extracellular domain) or antibody specifically binding the anti-CD33 CAR.

Cytotoxicity assays and cytokine excretion assays are performed using standard protocols. CD33 expressing cells, e.g., MOLM cells expressing luciferase, are incubated at different ratios with NK cells. Killing is monitored and calculated according to standard protocols. Cytokine assays are performed according to standard protocols.

HSC cells are differentiated into NK cells according to standard protocols. Expression of KLRK1 and anti-CD33 CAR is monitored during differentiation.

Mature NK cells transduced with the lentiviral vector demonstrate inducible expression of the anti-CD33 CAR, which can be induced by administration of doxycycline to the cells, and which is abolished upon withdrawal of doxycycline from the cells.

HSCs transduced with the lentiviral vector do not exhibit inducible expression of the anti-CD33 CAR, even in the presence of doxycycline. However, NK cells differentiated from these HSCs show inducible expression of the anti-CD33 CAR upon administration of doxycycline, which is abolished upon withdrawal of doxycycline from the cells.

When administered to a suitable mouse model, the transduced HSCs engraft and populate the hematopoietic niche. After a time sufficient for mature NK cells derived from these HSCs to emerge, anti-CD33 CAR expression can be induced in these mice by injection of doxycycline.

### Example 4: T-Cell Specific Expression of a CD33 CAR

A lentiviral expression vector for expression of a CD33 CAR in T-lymphocytes was designed in order to evaluate whether a CD3D promoter can drive T-cell specific expression of a transgene, here a CD33 CAR.

Briefly, the lentiviral vector comprises a pLVX vector backbone (Takara Bio USA) and an expression cassette comprising a nucleic acid sequence encoding a CD33 CAR under the control of a CD3D promoter.

Sequences used in the CD3D-CD33 CAR vector design are provided below:

Exemplary CD3D nucleic acid sequence
(SEQ ID NO: 13)
ggcgtgagacacggcacctggtttcactattcttttgagatcatgaaagaggcgcctggaaggcaataggtggaa
caattccgaggacaggccagcacattttcaggggccagagcccggactgaatgtccaggatctcaggcccctatc
tccctcagccagtcagtcagtgagccaacaaaaatgtgcagagctcccgttatgtgccaggcacaagatacagca
gtgagcaagacaggcatgactctgtccccaggtaagtgacagtctaactgcagcagcgtgaagagtttcagatcc
tcgtaggccacaggggaaaaaggtccatttgtctcaccgggaggctcaatattgcctgtcagtcaattatattat
tattaataactaccccttaatgagcacatatgatgcgtgaggcaccatgcaaatatcttgtttgaatcctaataa
tggtactaatggaataagactctgaaaaattttatggctggcccaggccccacaggtaggcagtattggacccag
gattcaaatctctggctggggtctctaaagcccaacctcccactgacaagaagctgctagatctggtgtccctgg
ctgcctagtgaagggtcctgagaaagatcagcctccatgagaaatctagctgctacggcttgcgctatggggccg
acggcttctctcaaggggcttcgagatgtggcagtgtttaggttgtgtgtaaatgtggttgcattgtcaataggg
acgctaaagttcaggccaccttttccatattctctgccagctccctgctcagagatagagcaatttacaccgctt
ccttcctaccctacccctagcccacccccactctgaaaatttcccaccatcaacggcagaaagcagagaagcaga
catcttctagttcctcccccactctcctctttccggtacctgtgagtcagctaggggagggcagctctcacccag
gctgatagttcggtgacctggcttt
Anti-CD33 CAR-encoding nucleotide sequence
(SEQ ID NO: 14)
atggctctgcccgtcacagctctgctgctgcctctggccctgctgctgcacgccgccagacctcaggtgcagctc
gtgcagagcggcgctgaggtgaagaaacctggcagcagcgtgaaggtgagctgcaaggcctccggctacaccttc
accgactacaacatgcactgggtgaggcaagcccctggccagggactggagtggatcggctacatctacccttac
aacggcggcacaggctacaaccagaagttcaagtccaaggccaccatcaccgccgatgagtccaccaataccgcc
tacatggagctcagcagcctgaggtccgaggacacagccgtctactactgcgccaggggcaggcccgctatggac
tactggggccagggcaccctggtgacagtgagctctggtggcggcggatccggcggcggcggcagcggcggcggc
ggctccgacattcagatgacccagagccctagcagcctgagcgcttccgtgggagacagggtgaccatcacatgc
agggcctccgagagcgtggacaattacggcatcagcttcatgaactggttccagcagaagcccggcaaggccccc
aaactgctgatctatgccgccagcaatcagggctccggcgtgcctagcaggttttccggcagcggcagcggcacc
gactttaccctgaccatctccagcctgcagcctgacgatttcgccacctactactgccagcagagcaaggaggtg
ccttggacctttggacagggcacaaaggtggagatcaagtccggagccgccgccatcgaagtgatgtacccccct
ccctacctggataacgagaagagcaacggcaccatcatccacgtgaagggaaagcacctgtgtcccagccccctg
tttcccggccctagcaagcccttctgggtgctggtggtggtcggcggagtgctggcctgctacagcctcctggtg
accgtggccttcatcatcttctgggtgaggagcaagaggtccaggctgctgcacagcgactacatgaatatgacc
cccagaaggcccggccccaccagaaagcactatcagccctacgccccccccagggactttgccgcctacaggagc
agggtgaagttcagcagatccgccgatgcccctgcttaccagcagggccagaaccagctgtataacgagctgaac
ctgggcaggagggaggaatacgacgtgctggataagaggaggggaagggaccccgagatgggcggaaagcccagg
aggaagaacccccaggagggcctgtacaatgagctgcagaaagacaagatggccgaggcctacagcgagatcggc
atgaagggcgagaggaggaggggcaagggccatgacggcctgtaccaaggcctgtccaccgccaccaaggatacc
tacgacgccctgcacatgcaggccctgcctcccaggggatcc
Anti-CD33 CAR amino acid sequence
(SEQ ID NO: 15)
MALPVTALLLPLALLLHAARPQVQLVQSGAEVKKPGSSVKVSCKASGYTFTDYNMHWVRQ
APGQGLEWIGYIYPYNGGTGYNQKFKSKATITADESTNTAYMELSSLRSEDTAVYYCARG
RPAMDYWGQGTLVTVSSGGGGSGGGGSGGGGSDIQMTQSPSSLSASVGDRVTITCRASES
VDNYGISFMNWFQQKPGKAPKLLIYAASNQGSGVPSRFSGSGSGTDFTLTISSLQPDDFA
TYYCQQSKEVPWTFGQGTKVEIKSGAAAIEVMYPPPYLDNEKSNGTIIHVKGKHLCPSPL
FPGPSKPFWVLVVVGGVLACYSLLVTVAFIIFWVRSKRSRLLHSDYMNMTPRRPGPTRKH
YQPYAPPRDFAAYRSRVKFSRSADAPAYQQGQNQLYNELNLGRREEYDVLDKRRGRDPEM
GGKPRRKNPQEGLYNELQKDKMAEAYSEIGMKGERRRGKGHDGLYQGLSTATKDTYDALH
MQALPPRGS
Lentiviral Vector Sequence
(SEQ ID NO: 16)
gggtctctctggttagaccagatctgagcctgggagctctctggctaactagggaacccactgcttaagcctcaa
taaagcttgccttgagtgcttcaagtagtgtgtgcccgtctgttgtgtgactctggtaactagagatccctcaga
cccttttagtcagtgtggaaaatctctagcagtggcgcccgaacagggacttgaaagcgaaagggaaaccagagg
agctctctcgacgcaggactcggcttgctgaagcgcgcacggcaagaggcgaggggcggcgactggtgagtacgc
caaaaattttgactagcggaggctagaaggagagagatgggtgcgagagcgtcagtattaagcgggggagaatta
gatcgcgatgggaaaaaattcggttaaggccagggggaaagaaaaaatataaattaaaacatatagtatgggcaa
gcagggagctagaacgattcgcagttaatcctggcctgttagaaacatcagaaggctgtagacaaatactgggac
agctacaaccatcccttcagacaggatcagaagaacttagatcattatataatacagtagcaaccctctattgtg
tgcatcaaaggatagagataaaagacaccaaggaagctttagacaagatagaggaagagcaaaacaaaagtaaga
ccaccgcacagcaagcggccgctgatcttcagacctggaggaggagatatgagggacaattggagaagtgaatta
tataaatataaagtagtaaaaattgaaccattaggagtagcacccaccaaggcaaagagaagagtggtgcagaga
gaaaaaagagcagtgggaataggagctttgttccttgggttcttgggagcagcaggaagcactatgggcgcagcg
tcaatgacgctgacggtacaggccagacaattattgtctggtatagtgcagcagcagaacaatttgctgagggct
attgaggcgcaacagcatctgttgcaactcacagtctggggcatcaagcagctccaggcaagaatcctggctgtg
gaaagatacctaaaggatcaacagctcctggggatttggggttgctctggaaaactcatttgcaccactgctgtg
ccttggaatgctagttggagtaataaatctctggaacagatttggaatcacacgacctggatggagtgggacaga
gaaattaacaattacacaagcttaatacactccttaattgaagaatcgcaaaaccagcaagaaaagaatgaacaa
gaattattggaattagataaatgggcaagtttgtggaattggtttaacataacaaattggctgtggtatataaaa
ttattcataatgatagtaggaggcttggtaggtttaagaatagtttttgctgtactttctatagtgaatagagtt
aggcagggatattcaccattatcgtttcagacccacctcccaaccccgaggggacccgacaggcccgaaggaata
gaagaagaaggtggagagagagacagagacagatccattcgattagtgaacggatctcgacggtatcgattagac
tgtagcccaggaatatggcagctagattgtacacatttagaaggaaaagttatcttggtagcagttcatgtagcc
agtggatatatagaagcagaagtaattccagcagagacagggcaagaaacagcatacttcctcttaaaattagca
ggaagatggccagtaaaaacagtacatacagacaatggcagcaatttcaccagtactacagttaaggccgcctgt
tggtgggcggggatcaagcaggaatttggcattccctacaatccccaaagtcaaggagtaatagaatctatgaat
aaagaattaaagaaaattataggacaggtaagagatcaggctgaacatcttaagacagcagtacaaatggcagta
ttcatccacaattttaaaagaaaaggggggattggggggtacagtgcaggggaaagaatagtagacataatagca
acagacatacaaactaaagaattacaaaaacaaattacaaaaattcaaaattttcgggtttattacagggacagc
agagatccagtttggctgcatacgcgtcgtgaggcgtgagacacggcacctggtttcactattcttttgagatca
tgaaagaggcgcctggaaggcaataggtggaacaattccgaggacaggccagcacattttcaggggccagagccc
ggactgaatgtccaggatctcaggcccctatctccctcagccagtcagtcagtgagccaacaaaaatgtgcagag
ctcccgttatgtgccaggcacaagatacagcagtgagcaagacaggcatgactctgtccccaggtaagtgacagt
ctaactgcagcagcgtgaagagtttcagatcctcgtaggccacaggggaaaaaggtccatttgtctcaccgggag
gctcaatattgcctgtcagtcaattatattattattaataactaccccttaatgagcacatatgatgcgtgaggc
accatgcaaatatcttgtttgaatcctaataatggtactaatggaataagactctgaaaaattttatggctggcc
caggccccacaggtaggcagtattggacccaggattcaaatctctggctggggtctctaaagcccaacctcccac
tgacaagaagctgctagatctggtgtccctggctgcctagtgaagggtcctgagaaagatcagcctccatgagaa
atctagctgctacggcttgcgctatggggccgacggcttctctcaaggggcttcgagatgtggcagtgtttaggt
tgtgtgtaaatgtggttgcattgtcaatagggacgctaaagttcaggccaccttttccatattctctgccagctc
cctgctcagagatagagcaatttacaccgcttccttcctaccctacccctagcccacccccactctgaaaatttc
ccaccatcaacggcagaaagcagagaagcagacatcttctagttcctcccccactctcctctttccggtacctgt
gagtcagctaggggagggcagctctcacccaggctgatagttcggtgacctggctttgctagcctcgaggccacc
atggctctgcccgtcacagctctgctgctgcctctggccctgctgctgcacgccgccagacctcaggtgcagctc
gtgcagagcggcgctgaggtgaagaaacctggcagcagcgtgaaggtgagctgcaaggcctccggctacaccttc
accgactacaacatgcactgggtgaggcaagcccctggccagggactggagtggatcggctacatctacccttac
aacggcggcacaggctacaaccagaagttcaagtccaaggccaccatcaccgccgatgagtccaccaataccgcc
tacatggagctcagcagcctgaggtccgaggacacagccgtctactactgcgccaggggcaggcccgctatggac
tactggggccagggcaccctggtgacagtgagctctggtggcggcggatccggcggcggcggcagcggcggcggc
ggctccgacattcagatgacccagagccctagcagcctgagcgcttccgtgggagacagggtgaccatcacatgc
agggcctccgagagcgtggacaattacggcatcagcttcatgaactggttccagcagaagcccggcaaggccccc
aaactgctgatctatgccgccagcaatcagggctccggcgtgcctagcaggttttccggcagcggcagcggcacc
gactttaccctgaccatctccagcctgcagcctgacgatttcgccacctactactgccagcagagcaaggaggtg
ccttggacctttggacagggcacaaaggtggagatcaagtccggagccgccgccatcgaagtgatgtacccccct
ccctacctggataacgagaagagcaacggcaccatcatccacgtgaagggaaagcacctgtgtcccagccccctg
tttcccggccctagcaagcccttctgggtgctggtggtggtcggcggagtgctggcctgctacagcctcctggtg
accgtggccttcatcatcttctgggtgaggagcaagaggtccaggctgctgcacagcgactacatgaatatgacc
cccagaaggcccggccccaccagaaagcactatcagccctacgccccccccagggactttgccgcctacaggagc
agggtgaagttcagcagatccgccgatgcccctgcttaccagcagggccagaaccagctgtataacgagctgaac
ctgggcaggagggaggaatacgacgtgctggataagaggaggggaagggaccccgagatgggcggaaagcccagg
aggaagaacccccaggagggcctgtacaatgagctgcagaaagacaagatggccgaggcctacagcgagatcggc
atgaagggcgagaggaggaggggcaagggccatgacggcctgtaccaaggcctgtccaccgccaccaaggatacc
tacgacgccctgcacatgcaggccctgcctcccaggggatcctaatgatcagtcgacaatcaacctctggattac
aaaatttgtgaaagattgactggtattcttaactatgttgctccttttacgctatgtggatacgctgctttaatg
cctttgtatcatgctattgcttcccgtatggctttcattttctcctccttgtataaatcctggttgctgtctctt
tatgaggagttgtggcccgttgtcaggcaacgtggcgtggtgtgcactgtgtttgctgacgcaacccccactggt
tggggcattgccaccacctgtcagctcctttccgggactttcgctttccccctccctattgccacggcggaactc
atcgccgcctgccttgcccgctgctggacaggggctcggctgttgggcactgacaattccgtggtgttgtcgggg
aagctgacgtcctttccatggctgctcgcctgtgttgccacctggattctgcgcgggacgtccttctgctacgtc
ccttcggccctcaatccagcggaccttccttcccgcggcctgctgccggctctgcggcctcttccgcgtcttcgc
cttcgccctcagacgagtoggatctccctttgggccgcctccccgcctggaattcgagctcggtacctttaagac
caatgacttacaaggcagctgtagatcttagccactttttaaaagaaaaggggggactggaagggctaattcact
cccaacgaagacaagatctgctttttgcttgtactgggtctctctggttagaccagatctgagcctgggagctct
ctggctaactagggaacccactgcttaagcctcaataaagcttgccttgagtgcttcaagtagtgtgtgcccgtc
tgttgtgtgactctggtaactagagatccctcagacccttttagtcagtgtggaaaatctctagca

Annotation of certain features within the vector sequence above:

| Feature | Nucleotide position numbers |
|---|---|
| 5′ LTR | 1 . . . 181 |
| -psi (Packaging signal) | 228 . . . 353 |
| RRE | 846 . . . 1079 |
| cPPT/CTS | 1962 . . . 2079 |
| CD3D promoter | 2133 . . . 3132 |
| Kozak sequence | 3145 . . . 3150 |
| Anti CD33 CAR | 3151 . . . 4617 |
| WPRE | 4633 . . . 5221 |
| 3′ LTR | 5308 . . . 5541 |

The lentiviral vector was cloned into a cloning plasmid for amplification in bacteria. Amplification, purification of the lentiviral fragment, lentiviral packaging and concentration, and transduction of target cells was performed according to standard methods.

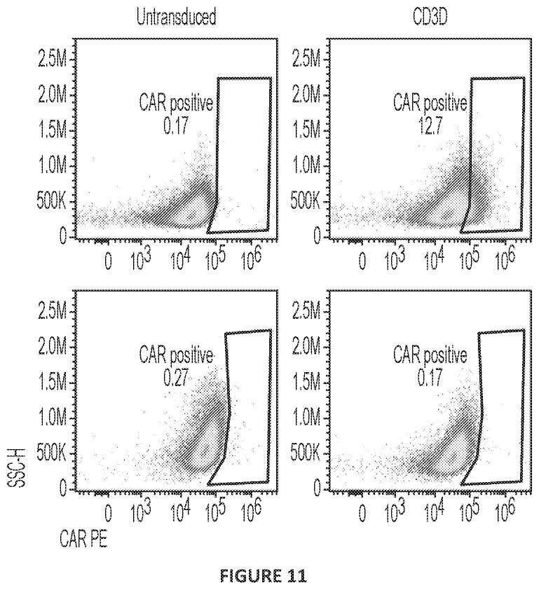

Jurkat cells and Raji cells were transduced with lentiviral supernatant containing the exemplary construct containing the CD3D promoter. Fluorescence-activated cell sorting (FACS) analysis was used to quantify the number of cells expressing the CD33 CAR (CAR positive cells) (FIG. 11). The construct placing CD33 CAR under the exemplary CD3D promoter produced T cell-specific CAR expression at a frequency of 12.7% in Jurkat cells, which exhibit similar expression patterns to T cells. Expression in Raji cells was very low, which is consistent with the selected promoter not being active in undifferentiated hematopoietic cells. The results show that exemplary cell type specific promoters of the disclosure can be used to express a transgene, e.g., a CAR or a transactivator, in a cell type specific manner.

### REFERENCES

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

What is claimed is:

1. A nucleic acid construct, comprising
(a) a first expression cassette comprising a nucleic acid encoding a tetracycline-responsive transactivator under the control of a cell type-specific promoter; and
(b) a second expression cassette comprising a nucleic acid encoding a transgene under the control of a tetracycline-responsive promoter.

2. The nucleic acid construct of claim 1, wherein the transgene encodes a chimeric antigen receptor.

3. The nucleic acid construct of claim 1 or 2, wherein the cell type-specific promoter is a constitutive promoter.

4. The nucleic acid construct of any one of claims 1-3, wherein the cell type specific promoter is a CD8 promoter.

5. The nucleic acid construct of any one of claims 1-3, wherein the cell type-specific promoter is a CD3delta promoter.

6. The nucleic acid construct of any one of claims 1-3, wherein the cell type-specific promoter is a CD56 promoter.

7. The nucleic acid construct of any one of claims 1-3, wherein the cell type-specific promoter is a CD244 promoter.

8. The nucleic acid construct of any one of claims 1-3, wherein the cell type-specific promoter is a CD94 promoter.

9. The nucleic acid construct of any one of claims 1-3, wherein the cell type-specific promoter is an NKG2D promoter.

10. The nucleic acid construct of any one of claims 1-3, wherein the cell type-specific promoter is a CD3D promoter.

11. The nucleic acid construct of any one of claims 1-3, wherein the cell type-specific promoter is a CD3E promoter.

12. The nucleic acid construct of any one of claims 1-11, wherein the tetracycline-responsive transactivator is an rtTA protein.

13. The nucleic acid construct of any one of claims 1-12, wherein the tetracycline-responsive transactivator is an rtTA-V16 protein or an rtT1-3G protein.

14. The nucleic acid construct of any one of claims 1-12, wherein the tetracycline-responsive transactivator is an rtTA2^{S}-M2 protein.

15. The nucleic acid construct of any one of claims 1-12, wherein the tetracycline-responsive transactivator is an rtTA-V10 protein.

16. The nucleic acid construct of any one of claims 1-15, wherein the transgene encodes a chimeric antigen receptor comprising
(i) a binding domain,
(ii) a hinge domain,
(iii) a transmembrane domain,
(iv) at least one co-stimulatory domain,
(v) a cytoplasmic signaling domain, or
(vi) a combination thereof.

17. The nucleic acid construct of claim 16, wherein the binding domain comprises an antibody, or an antigen-binding antibody fragment, that binds an antigen.

18. The nucleic acid construct of claim 16 or 17, wherein the binding domain comprises an scFv or a single domain antibody that binds to an antigen.

19. The nucleic acid construct of claim 17 or 18, wherein the antigen is a lineage-specific cell-surface antigen.

20. The nucleic acid construct of any one of claims 17-19, wherein expression of the antigen is associated with a neoplastic or malignant disease.

21. The nucleic acid construct of any one of claims 17-20, wherein the antigen is CD33, CD123, CD19, or CLL-1.

22. The nucleic acid construct of any one of claims 16-21, wherein the hinge domain of (ii) is a CD8a (CD8alpha) hinge domain.

23. The nucleic acid construct of any one of claims 16-22, wherein the transmembrane domain of (iii) is a CD8 or CD28 transmembrane domain.

24. The nucleic acid construct of any one of claims 16-23, wherein the costimulatory domain of (iv) is a 4-1BB or CD28 costimulatory domain, or a combination thereof.

25. The nucleic acid construct of any one of claims 16-24, wherein the cytoplasmic signaling domain of (v) is a CD3 (CD3zeta) cytoplasmic signaling domain.

26. A cell comprising the nucleic acid construct of any one of claims 1-25.

27. The cell of claim 26, wherein the cell is a hematopoietic stem or progenitor cell.

28. The cell of claim 26, wherein the cell is an immune effector cell.

29. The cell of claim 26 or 28, wherein the cell is a T-cell.

30. The cell of any one of claim 26, 28, or 29, wherein the cell is an alpha/beta T-cell.

31. The cell of any one of claim 26, 28, or 29, wherein the cell is a gamma/delta T-cell.

32. The cell of claim 26 or 28, wherein the cell is an NK cell.

33. A method, comprising administering a plurality of the cells of any one of claims 26-32 to a subject in need thereof.

34. The method of claim 33, wherein the subject has been diagnosed with or has a neoplastic or malignant disease, and wherein the transgene encoded by the nucleic acid construct comprised in the cell is a CAR targeting an antigen associated with the neoplastic or malignant disease.

35. The method of claim 33 or 34, wherein the cells are CAR-T cells.

36. The method of claim 33 or 34, wherein the cells are CAR-NK cells.

37. The method of claim 33 or 34, wherein the cells are HSCs or HPCs.

38. The method of any one of claims 33-37, further comprising administering to the subject an effective amount of tetracycline resulting in induction of the expression of the transgene.

39. The method of any one of claims 33-38, further comprising measuring expression of the transgene in a cell obtained from the subject.

40. The method of any one of claims 33-39, further comprising monitoring at least one symptom of the neoplastic or malignant disease and continuing administration of the tetracycline until the at least one symptom of the neoplastic or malignant disease is ameliorated.


SEQUENCE LISTING (16 sequences)

SEQ ID NO: 1 - DNA, 379 bp, Artificial Sequence
  Synthetic
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 2 - DNA, 1467 bp, Artificial Sequence
  Synthetic
atggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctga

SEQ ID NO: 3 - protein, 488 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 122 bp, Artificial Sequence
  Synthetic
aacttgtttattgcagcttataatggttacaaataaagcaatagcatcacaaatttcacaaataaagcatttttttcactgcattctagttgtggtttgtccaaactcatcaatgtatctta

SEQ ID NO: 5 - DNA, 1000 bp, Artificial Sequence
  Synthetic
ttctacccaccattaaagctttggcattctggccatttctttctttttaaaaaattattattattattttttattttgagatgggagtctcgctctatcccccagcctggagtgcagtgactcgatctccgctcgctgcaacttccgcctccagggttcaaacgattcttctgcctcagcctccccagtagctaggactacaggcttgcgccatcacgcccggctagttttttttttgttttttgtttgtttgtttgtttgtttttaaagtagaaacaggatttcgccatattggccaggctggtctcgaactcctgacctcagttgatccgaccgcctcggcctcccaaagtgttggaattacagcagtgagccaccgcgcaccaagcccggcccattctggccatatctatagacggatgcattgcacaggaggctcagcactaatcggtagatactgcgagatgctgggaggttaaggggcctacccgcaatatctctggccaatgccttgggctagaaatgccataattagccgctcttttgatcccttgcaaaatgcgaatcccaccgcacctccaccccacccgagtggtaatctcctagtggtaatctaagtgagcctgtgataagataagtagctcctggtggtgagggtgagaaattggggagctggagccccagccagggacgaggctgtaggggctagggcgaagatggaggctgctgggcccccagatggaagacggtaacgtgcgcccgcttcgtttttgctcgaggtcagtcaggtgcagactgaattcgaagtcgctccctcctccgctcaaccccgaccaggccaaaactaaagcagcaccgccccctgctgggccgacagggcatcagattttgctggacgcgggtgacaggcgagatagggagtgtccctgctgctagtgcccctgctgctagtgcctagttacctgcagtacttaccctccctgggcctcagtttttcttc

SEQ ID NO: 6 - DNA, 1382 bp, Artificial Sequence
  Synthetic
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaaccatggctgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 7 - protein, 248 aa, Artificial Sequence
  Synthetic
MSRLDKSKVINSALELLNGVGIEGLTTRKLAQKLGVEQPTLYWHVKNKRALLDALPIEMLDRHHTHSCPLEGESWQDFLRNNAKSYRCALLSHRDGAKVHLGTRPTEKQYETLENQLAFLCQQGFSLENALYALSAVGHFTLGCVLEEQEHQVAKEERETPTTDSMPPLLKQAIELFDRQGAEPAFLFGLELIICGLEKQLKCESGGPTDALDDFDLDMLPADALDDFDLDMLPADALDDFDLDMLPG

SEQ ID NO: 8 - DNA, 6940 bp, Artificial Sequence
  Synthetic
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagctaagatacattgatgagtttggacaaaccacaactagaatgcagtgaaaaaaatgctttatttgtgaaatttgtgatgctattgctttatttgtaaccattataagctgcaataaacaagttagagatccagtttatttcagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgcttgtacgcgggggcgtctgcgctcctgctgaacttcactctcagttcacatcctccttcttcttcttctggaaatcggcagctacagccatcttcctcttgagtagtttgtactggtctcataaatggttgtttgaatatatacaggagtttctttctgccccgtttgcagtaaagggtgataaccagtgacaggagaaggaccccacaagtcccggccaagggcgcccagatgtagatatcacaggcgaagtccagccccctcgtgtgcactgcgccccccgccgctggccggcacgcctctgggcgcagggacaggggctgcgacgcgatggtgggcgccggtgttggtggtcgcggcgctggcgtcgtggttccggacttgatctccacctttgtgccctgtccaaaggtccaaggcacctccttgctctgctggcagtagtaggtggcgaaatcgtcaggctgcaggctggagatggtcagggtaaagtcggtgccgctgccgctgccggaaaacctgctaggcacgccggagccctgattgctggcggcatagatcagcagtttgggggccttgccgggcttctgctggaaccagttcatgaagctgatgccgtaattgtccacgctctcggaggccctgcatgtgatggtcaccctgtctcccacggaagcgctcaggctgctagggctctgggtcatctgaatgtcggagccgccgccgccgctgccgccgccgccggatccgccgccaccagagctcactgtcaccagggtgccctggccccagtagtccatagcgggcctgcccctggcgcagtagtagacggctgtgtcctcggacctcaggctgctgagctccatgtaggcggtattggtggactcatcggcggtgatggtggccttggacttgaacttctggttgtagcctgtgccgccgttgtaagggtagatgtagccgatccactccagtccctggccaggggcttgcctcacccagtgcatgttgtagtcggtgaaggtgtagccggaggccttgcagctcaccttcacgctgctgccaggtttcttcacctcagcgccgctctgcacgagctgcacctgaggtctggcggcgtgcagcagcagggccagaggcagcagcagagctgtgacgggcagagccatggtggcttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactccgatggatctatttccggtgaattccgtagatactgcgagatgctgggaggttaaggggcctacccgcaatatctctggccaatgccttgggctagaaatgccataattagccgctcttttgatcccttgcaaaatgcgaatcccaccgcacctccaccccacccgagtggtaatctcctagtggtaatctaagtgagcctgtgataagataagtagctcctggtggtgagggtgagaaattggggagctggagccccagccagggacgaggctgtaggggctagggcgaagatggaggctgctgggcccccagatggaagacggtaacgtgcgcccgcttcgtttttgctcgaggtcagtcaggtgcagactgaattcgaagtcgctccctcctccgctcaaccccgaccaggccaaaactaaagcagcaccgccccctgctgggccgacagggcatcagattttgctggacgcgggtgacaggcgagatagggagtgtccctgctgctagtgcccctgctgctagtgcctagttacctgcagtacttaccctccctgggcctcagtttttcttctcgaggccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaggatcccgacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 9 - DNA, 1000 bp, Artificial Sequence
  Synthetic
gctttggttaatgggaagtggctaatggatagaggctgaaagaattgggaggagcagactagcaaaagtctagattcctgaaaacagagcataaagggcaattctggtgaaggctcagggggaaatgaggaacaaggtattgaaaatcgaagtaaatgccatccttgttgtaagtagcaaaaaccttggcaaaattgtgtcctgttctagaactttatggaatataaaaattatgagccatttgctagtatatctgctgaaagaaatatctaagaggcaaagcattcaggctactgtgtgactacttttagccaccaggaaatttaacccagcaagaagggagccatgggaatagattttgcaaaccagcacagatggtgactctacctccctctgctgtcctgtctcccataagccaaactctgtgctgtgagctgttaaagtccaagaattatttctggggaatctgtactcttaataatttacagacaacacaggtctgctttggatctgatcagacagactaaatcttggggactgtaccagtggccactgagaaaaggggctcggaatgttgtcttggatagaaaaatataataagaaacattggtgtgaatctagcatcagaaagatgacgtgaggacagcaaggaagagctggaaactgaggtttactcacaggctcctcaccctccgctgatgggcaggtgtgtgagctccagcatggagcaccacagcactaggtgggagaacggtgatagggtgatggggcagcctgtgagctggggcagtgtaggcagaggaggaactgtatcaccacagaaacttctgccttcacacatccctccagctaggcaggacaggtagagagtccagtgtcctggagcactagacctaaggaaggctgcatggggaggacaaaggacagtgacatcacaggatacccctcccatcaggaaaatcaaggcccagaactcactcggctcttccccaggagaacc

SEQ ID NO: 10 - DNA, 7390 bp, Artificial Sequence
  Synthetic
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagctaagatacattgatgagtttggacaaaccacaactagaatgcagtgaaaaaaatgctttatttgtgaaatttgtgatgctattgctttatttgtaaccattataagctgcaataaacaagttagagatccagtttatttcagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgcttgtacgcgggggcgtctgcgctcctgctgaacttcactctcagttcacatcctccttcttcttcttctggaaatcggcagctacagccatcttcctcttgagtagtttgtactggtctcataaatggttgtttgaatatatacaggagtttctttctgccccgtttgcagtaaagggtgataaccagtgacaggagaaggaccccacaagtcccggccaagggcgcccagatgtagatatcacaggcgaagtccagccccctcgtgtgcactgcgccccccgccgctggccggcacgcctctgggcgcagggacaggggctgcgacgcgatggtgggcgccggtgttggtggtcgcggcgctggcgtcgtggttccggacttgatctccacctttgtgccctgtccaaaggtccaaggcacctccttgctctgctggcagtagtaggtggcgaaatcgtcaggctgcaggctggagatggtcagggtaaagtcggtgccgctgccgctgccggaaaacctgctaggcacgccggagccctgattgctggcggcatagatcagcagtttgggggccttgccgggcttctgctggaaccagttcatgaagctgatgccgtaattgtccacgctctcggaggccctgcatgtgatggtcaccctgtctcccacggaagcgctcaggctgctagggctctgggtcatctgaatgtcggagccgccgccgccgctgccgccgccgccggatccgccgccaccagagctcactgtcaccagggtgccctggccccagtagtccatagcgggcctgcccctggcgcagtagtagacggctgtgtcctcggacctcaggctgctgagctccatgtaggcggtattggtggactcatcggcggtgatggtggccttggacttgaacttctggttgtagcctgtgccgccgttgtaagggtagatgtagccgatccactccagtccctggccaggggcttgcctcacccagtgcatgttgtagtcggtgaaggtgtagccggaggccttgcagctcaccttcacgctgctgccaggtttcttcacctcagcgccgctctgcacgagctgcacctgaggtctggcggcgtgcagcagcagggccagaggcagcagcagagctgtgacgggcagagccatggtggcttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactccgatggatctatttccggtgaattccgctttggttaatgggaagtggctaatggatagaggctgaaagaattgggaggagcagactagcaaaagtctagattcctgaaaacagagcataaagggcaattctggtgaaggctcagggggaaatgaggaacaaggtattgaaaatcgaagtaaatgccatccttgttgtaagtagcaaaaaccttggcaaaattgtgtcctgttctagaactttatggaatataaaaattatgagccatttgctagtatatctgctgaaagaaatatctaagaggcaaagcattcaggctactgtgtgactacttttagccaccaggaaatttaacccagcaagaagggagccatgggaatagattttgcaaaccagcacagatggtgactctacctccctctgctgtcctgtctcccataagccaaactctgtgctgtgagctgttaaagtccaagaattatttctggggaatctgtactcttaataatttacagacaacacaggtctgctttggatctgatcagacagactaaatcttggggactgtaccagtggccactgagaaaaggggctcggaatgttgtcttggatagaaaaatataataagaaacattggtgtgaatctagcatcagaaagatgacgtgaggacagcaaggaagagctggaaactgaggtttactcacaggctcctcaccctccgctgatgggcaggtgtgtgagctccagcatggagcaccacagcactaggtgggagaacggtgatagggtgatggggcagcctgtgagctggggcagtgtaggcagaggaggaactgtatcaccacagaaacttctgccttcacacatccctccagctaggcaggacaggtagagagtccagtgtcctggagcactagacctaaggaaggctgcatggggaggacaaaggacagtgacatcacaggatacccctcccatcaggaaaatcaaggcccagaactcactcggctcttccccaggagaacctcgaggccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaggatcccgacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 11 - DNA, 506 bp, Artificial Sequence
  Synthetic
tgttctcaaatgacttgagcccaggctgtttgcaaaatattgtaatttttcttctaacaatagtttttgaaagttatatagatacgtctatatgaagtttgttcaggtacttttatagtcaaattattctttctgcagaagttaattgcgttggttttatttcacttattagaaagtattttatctgaaaactatgctatgtccttttgcaattcacatagaataaaccagtacaaatgcccagcaaatccccaataagtttactatcatgactacagcaaagtgagtgtatgttcaacattgagaaagaaattggagttttaacatactatttctttatctaactaattgttttaacccgacatacaggacccattttagaattttcttctttaagaatcctttccacactctgtcagttggtttagggacctttatatatgacaataaaactctccctggtgcttagctttatcatggtatttatttcagaaatcttcttttag

SEQ ID NO: 12 - DNA, 6896 bp, Artificial Sequence
  Synthetic
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagctaagatacattgatgagtttggacaaaccacaactagaatgcagtgaaaaaaatgctttatttgtgaaatttgtgatgctattgctttatttgtaaccattataagctgcaataaacaagttagagatccagtttatttcagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgcttgtacgcgggggcgtctgcgctcctgctgaacttcactctcagttcacatcctccttcttcttcttctggaaatcggcagctacagccatcttcctcttgagtagtttgtactggtctcataaatggttgtttgaatatatacaggagtttctttctgccccgtttgcagtaaagggtgataaccagtgacaggagaaggaccccacaagtcccggccaagggcgcccagatgtagatatcacaggcgaagtccagccccctcgtgtgcactgcgccccccgccgctggccggcacgcctctgggcgcagggacaggggctgcgacgcgatggtgggcgccggtgttggtggtcgcggcgctggcgtcgtggttccggacttgatctccacctttgtgccctgtccaaaggtccaaggcacctccttgctctgctggcagtagtaggtggcgaaatcgtcaggctgcaggctggagatggtcagggtaaagtcggtgccgctgccgctgccggaaaacctgctaggcacgccggagccctgattgctggcggcatagatcagcagtttgggggccttgccgggcttctgctggaaccagttcatgaagctgatgccgtaattgtccacgctctcggaggccctgcatgtgatggtcaccctgtctcccacggaagcgctcaggctgctagggctctgggtcatctgaatgtcggagccgccgccgccgctgccgccgccgccggatccgccgccaccagagctcactgtcaccagggtgccctggccccagtagtccatagcgggcctgcccctggcgcagtagtagacggctgtgtcctcggacctcaggctgctgagctccatgtaggcggtattggtggactcatcggcggtgatggtggccttggacttgaacttctggttgtagcctgtgccgccgttgtaagggtagatgtagccgatccactccagtccctggccaggggcttgcctcacccagtgcatgttgtagtcggtgaaggtgtagccggaggccttgcagctcaccttcacgctgctgccaggtttcttcacctcagcgccgctctgcacgagctgcacctgaggtctggcggcgtgcagcagcagggccagaggcagcagcagagctgtgacgggcagagccatggtggcttacgagggtaggaagtggtacggaaagttggtataagacaaaagtgttgtggaattgctccaggcgatctgacggttcactaaacgagctctgcttttataggcgcccaccgtacacgcctaaagcttatacgttctctatcactgatagggagtaaactggatatacgttctctatcactgatagggagtaaactgtagatacgttctctatcactgatagggagtaaactggtcatacgttctctatcactgatagggagtaaactccttatacgttctctatcactgatagggagtaaagtctgcatacgttctctatcactgatagggagtaaactcttcatacgttctctatcactgatagggagtaaactccgatggatctatttccggtgaattcctgttctcaaatgacttgagcccaggctgtttgcaaaatattgtaatttttcttctaacaatagtttttgaaagttatatagatacgtctatatgaagtttgttcaggtacttttatagtcaaattattctttctgcagaagttaattgcgttggttttatttcacttattagaaagtattttatctgaaaactatgctatgtccttttgcaattcacatagaataaaccagtacaaatgcccagcaaatccccaataagtttactatcatgactacagcaaagtgagtgtatgttcaacattgagaaagaaattggagttttaacatactatttctttatctaactaattgttttaacccgacatacaggacccattttagaattttcttctttaagaatcctttccacactctgtcagttggtttagggacctttatatatgacaataaaactctccctggtgcttagctttatcatggtatttatttcagaaatcttcttttagtcgaggccaccatgtctagactggacaagagcaaagtcataaactctgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccactcctgccccctggaaggcgagtcatggcaagactttctgcggaacaacgccaagtcataccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgaaacaagcaattgagctgttcgaccggcagggagccgaacctgccttccttttcggcctggaactaatcatatgtggcctggagaaacagctaaagtgcgaaagcggcgggccgaccgacgcccttgacgattttgacttagacatgctcccagccgatgcccttgacgactttgaccttgatatgctgcctgctgacgctcttgacgattttgaccttgacatgctccccgggtaaggatcccgacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagt

SEQ ID NO: 13 - DNA, 1000 bp, Artificial Sequence
  Synthetic
ggcgtgagacacggcacctggtttcactattcttttgagatcatgaaagaggcgcctggaaggcaataggtggaacaattccgaggacaggccagcacattttcaggggccagagcccggactgaatgtccaggatctcaggcccctatctccctcagccagtcagtcagtgagccaacaaaaatgtgcagagctcccgttatgtgccaggcacaagatacagcagtgagcaagacaggcatgactctgtccccaggtaagtgacagtctaactgcagcagcgtgaagagtttcagatcctcgtaggccacaggggaaaaaggtccatttgtctcaccgggaggctcaatattgcctgtcagtcaattatattattattaataactaccccttaatgagcacatatgatgcgtgaggcaccatgcaaatatcttgtttgaatcctaataatggtactaatggaataagactctgaaaaattttatggctggcccaggccccacaggtaggcagtattggacccaggattcaaatctctggctggggtctctaaagcccaacctcccactgacaagaagctgctagatctggtgtccctggctgcctagtgaagggtcctgagaaagatcagcctccatgagaaatctagctgctacggcttgcgctatggggccgacggcttctctcaaggggcttcgagatgtggcagtgtttaggttgtgtgtaaatgtggttgcattgtcaatagggacgctaaagttcaggccaccttttccatattctctgccagctccctgctcagagatagagcaatttacaccgcttccttcctaccctacccctagcccacccccactctgaaaatttcccaccatcaacggcagaaagcagagaagcagacatcttctagttcctcccccactctcctctttccggtacctgtgagtcagctaggggagggcagctctcacccaggctgatagttcggtgacctggcttt

SEQ ID NO: 14 - DNA, 1467 bp, Artificial Sequence
  Synthetic
atggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcc

SEQ ID NO: 15 - protein, 489 aa, Artificial Sequence
  Synthetic
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 16 - DNA, 5541 bp, Artificial Sequence
  Synthetic
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcatacgcgtcgtgaggcgtgagacacggcacctggtttcactattcttttgagatcatgaaagaggcgcctggaaggcaataggtggaacaattccgaggacaggccagcacattttcaggggccagagcccggactgaatgtccaggatctcaggcccctatctccctcagccagtcagtcagtgagccaacaaaaatgtgcagagctcccgttatgtgccaggcacaagatacagcagtgagcaagacaggcatgactctgtccccaggtaagtgacagtctaactgcagcagcgtgaagagtttcagatcctcgtaggccacaggggaaaaaggtccatttgtctcaccgggaggctcaatattgcctgtcagtcaattatattattattaataactaccccttaatgagcacatatgatgcgtgaggcaccatgcaaatatcttgtttgaatcctaataatggtactaatggaataagactctgaaaaattttatggctggcccaggccccacaggtaggcagtattggacccaggattcaaatctctggctggggtctctaaagcccaacctcccactgacaagaagctgctagatctggtgtccctggctgcctagtgaagggtcctgagaaagatcagcctccatgagaaatctagctgctacggcttgcgctatggggccgacggcttctctcaaggggcttcgagatgtggcagtgtttaggttgtgtgtaaatgtggttgcattgtcaatagggacgctaaagttcaggccaccttttccatattctctgccagctccctgctcagagatagagcaatttacaccgcttccttcctaccctacccctagcccacccccactctgaaaatttcccaccatcaacggcagaaagcagagaagcagacatcttctagttcctcccccactctcctctttccggtacctgtgagtcagctaggggagggcagctctcacccaggctgatagttcggtgacctggctttgctagcctcgaggccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcctaatgatcagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca